(19) United States
(12) Patent Application Publication
(10) Pub. No.: US 2025/0003990 A1
(43) Pub. Date: Jan. 2, 2025

(54) ASSAYS, CARTRIDGES, AND KITS FOR DETECTION OF BIOMARKERS, INCLUDING BRAIN INJURY BIOMARKERS

(71) Applicant: Abbott Point of Care Inc., Princeton, NJ (US)

(72) Inventors: Kevin Joseph Duesling, Nepean (CA); Cassandra Leanne Scott, Nepean (CA); Emegbero John Omakor, Nepean (CA); Graham Edwin Garrett, Nepean (CA); Omatsone Cyril Boyo, Nepean (CA); Jinghua Hu, Nepean (CA); Matthew Thomas Clahane, Nepean (CA); James Tyler Kevin Smith, Nepean (CA)

(21) Appl. No.: 18/761,632

(22) Filed: Jul. 2, 2024

Related U.S. Application Data
(63) Continuation of application No. PCT/US24/26502, filed on Apr. 26, 2024.
(60) Provisional application No. 63/462,586, filed on Apr. 28, 2023.
(60) Provisional application No. 63/613,095, filed on Dec. 21, 2023.

(51) Int. Cl. G01N 33/68; C12Q 1/44; G01N 33/573; G01N 33/58

(52) U.S. Cl. CPC G01N 33/6896; C12Q 1/44; G01N 33/573; G01N 33/58; G01N 2333/47; G01N 2333/922; G01N 2470/04; G01N 2800/28

(57) ABSTRACT

Disclosed herein are improved assays, cartridges, kits, and methods of use thereof for detecting biomarkers, e.g., one or more biomarkers of brain injury, including, without limitation, biomarkers of acquired brain injury (ABI), such as, traumatic brain injury (TBI). The improved assays described herein may aid in the diagnosis and evaluation of a subject (e.g., a human subject) that has sustained or may have sustained an injury to the head (e.g., TBI) by detecting levels of a biomarker, such as UCH-L1, GFAP, or a combination thereof, in samples taken from a subject (e.g., a human subject).

### RELATED APPLICATION INFORMATION

The application is a continuation of International Application No. PCT/US24/26502, filed Apr. 26, 2024, which claims priority to U.S. Application No. 63/462,586, filed on Apr. 28, 2023, and U.S. Application No. 63/613,095, filed on Dec. 21, 2023, the contents of each of which are herein incorporated by reference.

### SEQUENCE LISTING STATEMENT

The contents of the electronic sequence listing titled ABBTL-43165-303-ST26.xml (Size: 7,937 bytes; and Date of Creation: May 30, 2024) is herein incorporated by reference in its entirety.

### TECHNICAL FIELD

The present disclosure relates to improved assays, cartridges, kits, and methods of use thereof for detecting biomarkers, such as one or more biomarkers of brain injury, including biomarkers of acquired brain injury (ABI), such as, traumatic brain injury (TBI). Specifically, the improved assays of the present disclosure involve detecting levels of a biomarker using a solution that comprises a nuclease, a solution that does not contain any heparin, or a solution that comprises a nuclease and does not contain any heparin.

### BACKGROUND

Assays, cartridges and kits that reliably detect biomarkers (e.g., brain injury biomarkers, as well as biomarkers for other diseases, disorders and conditions) are of value in the healthcare industry. More than 5 million mild traumatic brain injuries (TBIs) occur each year in the United States alone. Much of TBI evaluation and diagnosis still is based on subjective data. Unfortunately, objective measurements such as head CT and Glasgow Coma Scale (GCS) score are not very comprehensive or sensitive in evaluating mild TBI. Moreover, a head CT is unrevealing a vast majority of the time for mild TBI, is expensive, and exposes the patient to unnecessary radiation. Additionally, a negative head CT does not mean the patient has been cleared from having a concussion; rather it just means certain interventions, such as surgery is not warranted. Clinicians and patients need objective, reliable information to accurately evaluate this condition to promote appropriate triage and recovery. To date, limited data have been available for the use of UCH-L1 and GFAP in the acute care setting to aid in patient evaluation and management.

Mild TBI or concussion is much harder to objectively detect and presents an everyday challenge in emergency care units globally. Concussion usually causes no gross pathology, such as hemorrhage, and no abnormalities on conventional computed tomography scans of the brain, but rather rapid-onset neuronal dysfunction that resolves in a spontaneous manner over a few days to a few weeks. Approximately 15% of mild TBI patients suffer persisting cognitive dysfunction. There is an unmet need for detecting and assessing mild TBI victims on scene, in emergency rooms and clinics, in the sports area and in military activity (e.g., combat).

Current algorithms for assessment of the severity of brain injury include Glasgow Coma Scale score and other measures. These measures may at times be adequate for relating acute severity but are insufficiently sensitive for subtle pathology which can result in persistent deficit. GCS and other measures also do not enable differentiation among types of injury and may not be adequate. Thus, patients grouped into a single GCS level entering a clinical trial may have vastly heterogeneous severity and type of injury. Because outcomes also vary accordingly, inappropriate classification undermines the integrity of a clinical trial. Improved classification of injury will enable more precise delineation of disease severity and type for TBI patients in clinical trials.

Additionally, current brain injury trials rely on outcome measures such as Glasgow Outcome Scale Extended, which capture global phenomena but fail to assess for subtle differences in outcome. Sensitive outcome measures are needed to determine how well patients have recovered from brain injury in order to test therapeutics and prophylactics. Improved assays, cartridges, kits, and methods of use thereof for detecting biomarkers, such as one or more biomarkers of brain injury, including TBI biomarkers, would aid in TBI assessment, as well as assessment of other diseases, disorders and conditions.

### SUMMARY

In some embodiments, the present disclosure relates to a cartridge for use in a point-of-care device. The cartridge used in a point-of-care device comprises a housing. The housing contains a pre-treatment reagent for use in performing at least one assay to detect the presence of or determine the amount or concentration of at least one biomarker or analyte of interest. The pre-treatment reagent used in the cartridge comprises at least one nuclease. Additionally, the housing of the cartridge can further contain a biosensor chip, a sample chamber, and a waste container.

In some embodiments, the pre-treatment reagent in the cartridge is contained in a printed pre-treatment layer in the sample chamber. When the pre-treatment reagent is printed as a pre-treatment layer, the pre-treatment layer comprises about 1.5 U/mL of a nuclease. Additionally, in some further embodiments, the pre-treatment layer containing the pre-treatment reagent dissolves when contacted with a biological sample containing an analyte of interest. When the pre-treatment layer dissolves when contacted with the biological sample, the amount or concentration of nuclease dissolved in the biological sample is about 1.0 U/mL.

In some embodiments, the nuclease used in housing is a Benzonase® nuclease. Benzonase® nuclease is also known as a Serratia marcescens nuclease.

In some embodiments, the biomarker or analyte of interest is GFAP, UCH-L1, or GFAP and UCH-L1.

In yet further embodiments, the present disclosure relates to a kit containing the above described cartridge.

In other embodiments, the present disclosure relates to methods for measuring levels of a biomarker, (e.g., a ABI biomarker or a TBI biomarker) in a sample obtained from a subject. In some embodiments, the sample is obtained from the subject after an actual or suspected injury to the head. In some embodiments, the disclosure relates to methods for determining whether levels of an ABI biomarker, such as a TBI biomarker, are elevated in a sample obtained from a subject. In some embodiments, the biomarker for ABI is GFAP and/or UCH-L1. In some embodiments, the provided herein is a method for determining whether a subject's levels of GFAP, UCH-L1, or GFAP and UCH-L1 are elevated. In some embodiments, provided herein is an assay for measuring the amount or concentration of a ABI biomarker and, optionally, determining whether levels of a ABI biomarker are elevated in a sample obtained from a subject (e.g., such as in comparison to a reference level). In some embodiments, provided herein is a method comprising performing at least one assay for ubiquitin carboxy-terminal hydrolase L1 (UCH-L1), at least one assay for glial fibrillary acidic protein (GFAP), or at least one assay for UCH-L1 and GFAP in at least one sample obtained from a human subject (e.g. after an actual or suspected injury to the head). In some embodiments, the assay comprises contacting a sample, either simultaneously or sequentially, in any order, with: (1) a capture antibody which binds to an epitope on the ABI biomarker to form a capture antibody-biomarker-complex, (2) a detection antibody which binds to an epitope on the ABI biomarker that is not bound by the capture antibody to form a capture antibody-biomarker-detection antibody complex, and (3) a solution comprising at least one nuclease. In some embodiments the at least one nuclease is an endonuclease. In some embodiments, the at least one nuclease cleaves DNA (e.g. is a DNase). In some embodiments, the at least one nuclease cleaves RNA (e.g., is an RNase), but preferably is a nuclease that cleaves RNA (i.e., an RNase) also must have DNA cleavage activity (i.e., must also be a DNase). In some embodiments, the at least one nuclease cleaves DNA and RNA. In some embodiments, the solution comprising at least one nuclease does not contain heparin. Either the capture antibody or the detection antibody may comprise a detectable label. In some embodiments, the method comprises contacting the sample with the solution comprising at least one nuclease before contacting the sample with the capture antibody or the detection antibody (e.g., as a pre-treatment step). In some embodiments, the method comprises contacting the sample with the solution comprising at least one nuclease after contacting the sample with the capture antibody (e.g., after formation of the capture antibody-biomarker complex). In some embodiments, the method comprises contacting the sample with the solution comprising at least one nuclease after contacting the sample with the capture antibody and the detection antibody (e.g. after formation of the capture antibody-biomarker-detection antibody complex). In some embodiments, the method further comprises measuring the amount or concentration of the biomarker in the sample based upon the signal generated by the detectable label in the capture antibody-biomarker-detection antibody complex. In some embodiments, the solution comprising at least one nuclease comprises at least about 1 U/mL DNase. In other embodiments, the solution comprising a at least one nuclease comprises from about 1 U/mL DNase to about 1.5 U/mL DNase.

In some embodiments, the ABI biomarker is GFAP. Accordingly, in some embodiments the assay comprises contacting the sample, either simultaneously or sequentially, in any order, with: (1) a capture antibody which binds to an epitope on GFAP to form a capture antibody-GFAP antigen-complex, (2) a detection antibody which binds to an epitope on GFAP that is not bound by the capture antibody to form a capture antibody-GFAP antigen-detection antibody complex, and (3) a solution comprising at least one nuclease. Either the capture antibody or the detection antibody may comprise a detectable label. In some embodiments, the method comprises contacting the sample with the solution comprising at least one nuclease (e.g., an endonuclease) after contacting the sample with the capture antibody (e.g., after formation of the capture antibody-GFAP antigen-complex). In some embodiments, the method comprises contacting the sample with the solution comprising at least one nuclease (e.g., an endonuclease) after contacting the sample with the capture antibody and the detection antibody (e.g., after formation of the capture antibody-GFAP antigen-detection antibody complex). In some embodiments, the method further comprises measuring the amount or concentration of GFAP in the sample based upon the signal generated by the detectable label in the capture antibody-GFAP antigen-detection antibody complex. In some embodiments, the solution comprising at least one nuclease comprises at least about 1 U/mL DNase.

In some embodiments, the ABI biomarker is UCH-L1. Accordingly, in some embodiments the assay comprises contacting the sample, either simultaneously or sequentially, in any order, with: (1) a capture antibody which binds to an epitope on UCH-L1 to form a capture antibody-UCH-L1 antigen-complex, (2) a detection antibody which binds to an epitope on UCH-L1 that is not bound by the capture antibody to form a capture antibody-UCH-L1 antigen-detection antibody complex, and (3) a solution comprising at least one nuclease. Either the capture antibody or the detection antibody may comprise a detectable label. In some embodiments, the method comprises contacting the sample with the solution comprising at least one nuclease (e.g., an endonuclease) after contacting the sample with the capture antibody (e.g. after formation of the capture antibody-UCH-L1 antigen-complex). In some embodiments, the method comprises contacting the sample with the solution comprising at least one nuclease (e.g., an endonuclease) after contacting the sample with the capture antibody and the detection antibody (e.g., after formation of the capture antibody-UCH-L1 antigen-detection antibody complex). In some embodiments, the method further comprises measuring the amount or concentration of UCH-L1 in the sample based upon the signal generated by the detectable label in the capture antibody-UCH-L1 antigen-detection antibody complex. In some embodiments, the solution comprising at least one nuclease comprises at least about 1 U/mL DNase.

For any of the embodiments described herein, the sample can be obtained from the subject within about 48 hours after an actual or suspected injury to the head. For instance, in some embodiments the sample is obtained within about 24 hours after the actual or suspected injury to the head. In some embodiments, the sample is obtained within about 12 hours after the actual or suspected injury to the head. In some embodiments, the method further comprises determining whether the subject's levels of the ABI biomarker (e.g., GFAP, UCH-L1, or GFAP and UCH-L1) are elevated, not elevated, or that the assays for the TBI biomarker (e.g., assays for GFAP, UCH-L1, or GFAP and UCH-L1) need to be repeated.

In still yet further embodiments, the biomarker for TBI is GFAP and/or UCH-L1. In some embodiments, the provided herein is a method for determining whether a subject's levels of GFAP, UCH-L1, or GFAP and UCH-L1 are elevated. In some embodiments, provided herein is an assay for measuring the amount or concentration of a TBI biomarker and, optionally, determining whether levels of a TBI biomarker are elevated in a sample obtained from a subject (e.g., such as in comparison to a reference level). In some embodiments, provided herein is a method comprising performing at least one assay for ubiquitin carboxy-terminal hydrolase L1 (UCH-L1), at least one assay for glial fibrillary acidic protein (GFAP), or at least one assay for UCH-L1 and GFAP in at least one sample obtained from a human subject (e.g. after an actual or suspected injury to the head). In some embodiments, the assay comprises contacting a sample, either simultaneously or sequentially, in any order, with: (1) a capture antibody which binds to an epitope on the TBI biomarker to form a capture antibody-biomarker-complex, (2) a detection antibody which binds to an epitope on the TBI biomarker that is not bound by the capture antibody to form a capture antibody-biomarker-detection antibody complex, and (3) a solution comprising at least one nuclease. In some embodiments the at least one nuclease is an endonuclease. In some embodiments, the at least one nuclease cleaves DNA (e.g. is a DNase). In some embodiments, the at least one nuclease cleaves RNA (e.g., is an RNase), but preferably is a nuclease that cleaves RNA (i.e., an RNase) also must have DNA cleavage activity (i.e., must also be a DNase). In some embodiments, the at least one nuclease cleaves DNA and RNA. In some embodiments, the solution comprising at least one nuclease does not contain heparin. Either the capture antibody or the detection antibody may comprise a detectable label. In some embodiments, the method comprises contacting the sample with the solution comprising at least one nuclease before contacting the sample with the capture antibody or the detection antibody (e.g., as a pre-treatment step). In some embodiments, the method comprises contacting the sample with the solution comprising at least one nuclease after contacting the sample with the capture antibody (e.g., after formation of the capture antibody-biomarker complex). In some embodiments, the method comprises contacting the sample with the solution comprising at least one nuclease after contacting the sample with the capture antibody and the detection antibody (e.g. after formation of the capture antibody-biomarker-detection antibody complex). In some embodiments, the method further comprises measuring the amount or concentration of the biomarker in the sample based upon the signal generated by the detectable label in the capture antibody-biomarker-detection antibody complex. In some embodiments, the solution comprising at least one nuclease comprises at least about 1 U/mL DNase. In other embodiments, the solution comprising at least one nuclease comprises from about 1 U/mL DNase to about 1.5 U/mL DNase.

In some embodiments, the TBI biomarker is GFAP. Accordingly, in some embodiments the assay comprises contacting the sample, either simultaneously or sequentially, in any order, with: (1) a capture antibody which binds to an epitope on GFAP to form a capture antibody-GFAP antigen-complex, (2) a detection antibody which binds to an epitope on GFAP that is not bound by the capture antibody to form a capture antibody-GFAP antigen-detection antibody complex, and (3) a solution comprising at least one nuclease. Either the capture antibody or the detection antibody may comprise a detectable label. In some embodiments, the method comprises contacting the sample with the solution comprising at least one nuclease (e.g., an endonuclease) after contacting the sample with the capture antibody (e.g., after formation of the capture antibody-GFAP antigen-complex). In some embodiments, the method comprises contacting the sample with the solution comprising at least one nuclease (e.g., an endonuclease) after contacting the sample with the capture antibody and the detection antibody (e.g., after formation of the capture antibody-GFAP antigen-detection antibody complex). In some embodiments, the method further comprises measuring the amount or concentration of GFAP in the sample based upon the signal generated by the detectable label in the capture antibody-GFAP antigen-detection antibody complex. In some embodiments, the solution comprising at least one nuclease comprises at least about 1 U/mL DNase.

In some embodiments, the TBI biomarker is UCH-L1. Accordingly, in some embodiments the assay comprises contacting the sample, either simultaneously or sequentially, in any order, with: (1) a capture antibody which binds to an epitope on UCH-L1 to form a capture antibody-UCH-L1 antigen-complex, (2) a detection antibody which binds to an epitope on UCH-L1 that is not bound by the capture antibody to form a capture antibody-UCH-L1 antigen-detection antibody complex, and (3) a solution comprising at least one nuclease. Either the capture antibody or the detection antibody may comprise a detectable label. In some embodiments, the method comprises contacting the sample with the solution comprising at least one nuclease (e.g., an endonuclease) after contacting the sample with the capture antibody (e.g. after formation of the capture antibody-UCH-L1 antigen-complex). In some embodiments, the method comprises contacting the sample with the solution comprising at least one nuclease (e.g., an endonuclease) after contacting the sample with the capture antibody and the detection antibody (e.g., after formation of the capture antibody-UCH-L1 antigen-detection antibody complex). In some embodiments, the method further comprises measuring the amount or concentration of UCH-L1 in the sample based upon the signal generated by the detectable label in the capture antibody-UCH-L1 antigen-detection antibody complex. In some embodiments, the solution comprising at least one nuclease comprises at least about 1 U/mL DNase.

For any of the embodiments described herein, the sample can be obtained from the subject within about 48 hours after an actual or suspected injury to the head. For instance, in some embodiments the sample is obtained within about 24 hours after the actual or suspected injury to the head. In some embodiments, the sample is obtained within about 12 hours after the actual or suspected injury to the head. In some embodiments, the method further comprises determining whether the subject's levels of the TBI biomarker (e.g., GFAP, UCH-L1, or GFAP and UCH-L1) are elevated, not elevated, or that the assays for the TBI biomarker (e.g., assays for GFAP, UCH-L1, or GFAP and UCH-L1) need to be repeated.

In some embodiments, the method optionally comprises determining that the subject's levels of the TBI biomarker (e.g., GFAP, UCH-L1, or GFAP and UCH-L1) are elevated. The subject's levels of GFAP, UCH-L1, or GFAP and UCH-L1 are determined to be elevated when (i) the level of GFAP alone in the sample is equal to or above about 30 μg/mL, or equal to or above about 65 μg/mL (for example, for a point-of-care assay on a whole blood sample); (ii) the level of GFAP in the sample is equal to or above about 30 μg/mL, or equal to or above about 65 μg/mL (for example, for a point-of-care assay on a whole blood sample) and level of UCH-L1 in the sample is below about 360 μg/mL, cannot be determined or is not reported; (iii) the level of GFAP in the sample is equal to or above about 30 μg/mL, or equal to or above about 65 μg/mL (for example, for a point-of-care assay on a whole blood sample), and level of UCH-L1 in the sample is equal to or above about 360 μg/mL; (iv) the level of UCH-L1 alone in the sample is equal to or above about 360 μg/mL; or (v) the level of GFAP in the sample cannot be determined or is not reported and the level of UCH-L1 in the sample is equal to or above about 360 μg/mL.

In some embodiments, the method comprises determining that the subject's levels of the TBI biomarker (e.g., GFAP, UCH-L1, or GFAP and UCH-L1) are not elevated. The subject's levels of GFAP, UCH-L1, or UCH-L1 and GFAP are not elevated when the subject's levels of GFAP alone in the sample are below about 30 μg/mL, or below about 65 μg/mL (for example, for a point-of-care assay on a whole blood sample), levels of UCH-L1 alone in the sample are below about 360 μg/mL, or when the subject's levels of GFAP in the sample are below about 30 μg/mL, or below about 65 μg/mL (for example, for a point-of-care assay on a whole blood sample) and level of UCH-L1 in the sample are below about 360 μg/mL.

In some embodiments, the method comprises determining that assays for the TBI biomarker (e.g., UCH-L1, GFAP, or UCH-L1 and GFAP) should be repeated. For example, in some embodiments assays for UCH-L1, GFAP, or UCH-L1 and GFAP should be repeated when (i) the level of UCH-L1 alone in the sample cannot be determined or is not reported; (ii) the level of GFAP in the sample is below about 30 μg/mL, or below about 65 μg/mL (for example, for a point-of-care assay on a whole blood sample), and the level of UCH-L1 in the sample cannot be determined or is not reported; (iii) the level of GFAP alone in the sample cannot be determined or is not reported; (iv) the level of GFAP in the sample cannot be determined or is not reported and the level of UCH-L1 in the sample is below about 360 μg/mL; or (v) the level of GFAP in the sample cannot be determined or is not reported and the level of UCH-L1 in the sample cannot be determined or is not reported. In some embodiments, the method comprises communicating the determination on or from at least one instrument.

In some embodiments, the method further comprises performing a head computed tomography (CT) scan, magnetic resonance imaging (MRI) procedure, or both a CT scan or a MRI procedure on the subject when the subject's levels of the TBI biomarker (e.g., GFAP, UCH-L1, or GFAP and UCH-L1) are elevated. In some embodiments, the method further comprises determining not to perform a head computed tomography (CT) scan, magnetic resonance imaging (MRI) procedure, or both a head CT scan or a MRI procedure on the subject when the subject's levels of an ABI biomarkers, such as a TBI biomarker (e.g., GFAP, UCH-L1, or GFAP and UCH-L1) are not elevated.

In some embodiments, the method further comprises diagnosing the subject as having a traumatic brain injury (TBI) based upon the level of the TBI biomarker in the sample. In some embodiments, the method comprises diagnosing the subject as having a TBI when the level of GFAP alone is equal to or above about 30 μg/mL or 65 μg/mL, the level of UCH-L1 alone is equal to or above about 360 μg/mL, or the level of GFAP is equal to or above about 30 μg/mL or 65 μg/mL and/or the level of UCH-L1 is equal to or above about 360 μg/mL, regardless of whether a head CT scan is negative for a TBI or whether any head CT scan is performed.

In some embodiments, the method further comprises treating the subject for a mild, moderate, moderate to severe, or severe TBI when the subject's levels of GFAP, UCH-L1, or GFAP and UCH-L1 are elevated. In some embodiments, the method further comprises monitoring the subject when the subject's levels of GFAP, UCH-L1, or GFAP and UCH-L1 are elevated.

In some embodiments, the sample is taken within about 5 minutes, within about 10 minutes, within about 12 minutes, within about 15 minutes, within about 20 minutes, within about 30 minutes, within about 60 minutes, within about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours or within about 48 hours after the actual or suspected injury to the head.

In other embodiments, the sample is taken within about 8 hours to within about 48 hours after the actual or suspected injury to the head. In still other embodiments, the sample is taken within about 9 hours to within about 48 hours after the actual or suspected injury to the head. In still other embodiments, the sample is taken within about 10 hours to within about 48 hours after the actual or suspected injury to the head. In still other embodiments, the sample is taken within about 11 hours to within about 48 hours after the actual or suspected injury to the head. In still other embodiments, the sample is taken within about 12 hours to within about 48 hours after the actual or suspected injury to the head. In still other embodiments, the sample is taken within about 13 hours to within about 48 hours after the actual or suspected injury to the head. In still other embodiments, the sample is taken within about 14 hours to within about 48 hours after the actual or suspected injury to the head. In still other embodiments, the sample is taken within about 15 hours to within about 48 hours after the actual or suspected injury to the head. In still other embodiments, the sample is taken within about 16 hours to within about 48 hours after the actual or suspected injury to the head. In still other embodiments, the sample is taken within about 17 hours to within about 48 hours after the actual or suspected injury to the head. In still other embodiments, the sample is taken within about 18 hours to within about 48 hours after the actual or suspected injury to the head. In still other embodiments, the sample is taken within about 19 hours to within about 48 hours after the actual or suspected injury to the head. In still other embodiments, the sample is taken within about 20 hours to within about 48 hours after the actual or suspected injury to the head. In still other embodiments, the sample is taken within about 21 hours to within about 48 hours after the actual or suspected injury to the head. In still other embodiments, the sample is taken within about 22 hours to within about 48 hours after the actual or suspected injury to the head. In still other embodiments, the sample is taken within about 23 hours to within about 48 hours after the actual or suspected injury to the head. In still other embodiments, the sample is taken within about 24 hours to within about 48 hours after the actual or suspected injury to the head. In still other embodiments, the sample is taken within about 25 hours to within about 48 hours after the actual or suspected injury to the head. In still other embodiments, the sample is taken within about 26 hours to within about 48 hours after the actual or suspected injury to the head. In still other embodiments, the sample is taken within about 27 hours to within about 48 hours after the actual or suspected injury to the head. In still other embodiments, the sample is taken within about 28 hours to within about 48 hours after the actual or suspected injury to the head. In still other embodiments, the sample is taken within about 29 hours to within about 48 hours after the actual or suspected injury to the head. In still other embodiments, the sample is taken within about 30 hours to within about 48 hours after the actual or suspected injury to the head. In still other embodiments, the sample is taken within about 31 hours to within about 48 hours after the actual or suspected injury to the head. In still other embodiments, the sample is taken within about 32 hours to within about 48 hours after the actual or suspected injury to the head. In still other embodiments, the sample is taken within about 33 hours to within about 48 hours after the actual or suspected injury to the head. In still other embodiments, the sample is taken within about 34 hours to within about 48 hours after the actual or suspected injury to the head. In still other embodiments, the sample is taken within about 35 hours to within about 48 hours after the actual or suspected injury to the head. In still other embodiments, the sample is taken within about 36 hours to within about 48 hours after the actual or suspected injury to the head. In still other embodiments, the sample is taken within about 37 hours to within about 48 hours after the actual or suspected injury to the head. In still other embodiments, the sample is taken within about 38 hours to within about 48 hours after the actual or suspected injury to the head. In still other embodiments, the sample is taken within about 39 hours to within about 48 hours after the actual or suspected injury to the head. In still other embodiments, the sample is taken within about 40 hours to within about 48 hours after the actual or suspected injury to the head.

In some embodiments, the biomarker for TBI is GFAP. Accordingly, in some embodiments the methods described herein comprise performing an assay for GFAP. In some embodiments, the biomarker for TBI is UCH-L1. Accordingly, in some embodiments, the methods described herein comprise performing an assay for UCH-L1. In some embodiments, multiple biomarkers for TBI are assessed in the methods described herein. In some embodiments, the methods described herein comprise performing at least one assay for GFAP and/or at least one assay for UCH-L1. The at least one assay for UCH-L1 and/or at least one assay for GFAP can be performed simultaneously or sequentially, in any order.

In some embodiments, the sample is obtained a variety of ways. For example, the sample can be obtained after the subject sustained a head injury caused by physical shaking, blunt impact by an external mechanical or other force that results in a closed or open head trauma, one or more falls, explosions or blasts or other types of blunt force trauma. Alternatively, the sample can be obtained after the subject has ingested or been exposed to a fire, a chemical, toxin or combination of a chemical and toxin. Examples of chemicals or toxins are mold, asbestos, a pesticide, an insecticide, an organic solvent, a paint, a glue, a gas, an organic metal, a drug of abuse or one or more combinations thereof. Still further, the sample can be obtained from a subject that suffers from an autoimmune disease, a metabolic disorder, a brain tumor, hypoxia, a viral infection (e.g., SARS-CoV-2), a fungal infection, a bacterial infection, meningitis, hydrocephalus, or any combinations thereof.

In some embodiments, the assay (e.g., the assay for GFAP and/or the assay for UCH-L1) is an immunoassay or a clinical chemistry assay. In some embodiments, the assay is a single molecule detection assay or a point-of-care assay. In some embodiments, the amount of the at least one sample is about 10 μL to about 30 μL. For example, in some embodiments the amount of the at least one sample is about 20 μL.

In some embodiments, the assay (e.g., the at least one assay for UCH-L1, at least one assay for GFAP, or at least one assay for UCH-L1 and at least one assay for GFAP) is performed in about 10 to about 20 minutes. For example, in some embodiments the assay (e.g., the at least one assay for UCH-L1, at least one assay for GFAP, or at least one assay for UCH-L1 and at least one assay for GFAP) is performed in about 15 minutes.

In some embodiments, the subject has sustained an orthopedic injury in addition to an actual or suspected injury to the head. In some embodiments, the orthopedic injury and the injury to the head may have occurred simultaneously.

In some embodiments, the sample is selected from the group consisting of a whole blood sample, a capillary blood sample, a serum sample, a cerebrospinal fluid sample, a mixed sample of venous and capillary blood, a mixed sample of capillary blood and interstitial fluid, a tissue sample, a bodily fluid, and a plasma sample.

In still yet further embodiments, the present disclosure relates to a kit for performing an assay for a biomarker or analyte of interest. In some embodiments, the kit comprises a capture antibody, a detection antibody, and a solution that comprises at least one nuclease, wherein use of the kit for an assay for detecting one or more biomarkers or analytes of interest reduces (i) non-specific conjugate binding during the assay, (ii) formation of aggregates (such as DNA aggregates) during the assay; or (iii) a combination of (i) and (ii) when compared to an assay conduced using a kit that does not comprise the at least one nuclease. In some embodiments, the biomarker or analyte of interest is GFAP, UCH-L1 or GFAP and UCH-L1

In yet still further embodiments, the present disclosure relates to the use of a nuclease in preparation of a kit for measuring a level of a biomarker in a biological sample. In some embodiments, the biomarker is GFAP. In other embodiments, the biomarker is UCH-L1. In still wherein the nuclease used to prepare the kit is in the form of a solution. In still further embodiments, the solution contains at least 1 U/mL of the nuclease. In still yet further embodiments, the solution comprises about 1 U/mL to about 1.5 U/mL of the nuclease. In still yet further embodiments, the at least one nuclease is an endonuclease. In still yet further embodiments, the at least one nuclease is a Benzonase® nuclease. In yet further embodiments, the solution containing the nuclease further comprises at least one salt. In still further aspects, the at least one salt is magnesium chloride.

### BRIEF DESCRIPTION OF THE DRA WINGS

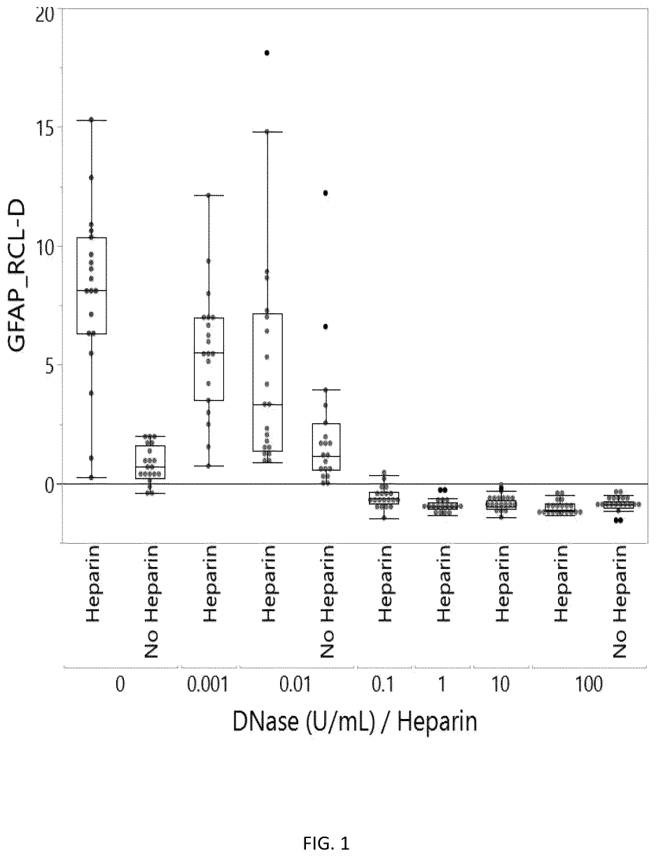

FIG. 1 shows a comparison of solutions containing various concentrations of nuclease with and without heparin on GFAP assay performance.

### DETAILED DESCRIPTION

In some embodiments, the present disclosure relates to a cartridge for use in a point-of-care device. The cartridge used in the point-of-care device comprises a housing. The housing contains a pre-treatment reagent for use in performing at least one assay to detect the presence of or determine the amount or concentration of at least one biomarker or analyte of interest. The pre-treatment reagent used in the cartridge comprises at least one nuclease. Additionally, the housing of the cartridge can further contain a biosensor chip, a sample chamber, and a waste container.

In other embodiments, the present disclosure relates to improved methods for detecting one or more biomarkers in a biological sample obtained from a subject. In some embodiments, the improved methods described herein can be used to aid in the diagnosis and evaluation of a subject (e.g., a human subject) that has or may have sustained an acquired brain injury (ABI), such as, for example, a mild, moderate, severe, or moderate to severe traumatic brain injury (TBI), using one or more biomarkers, such as one or more biomarkers of TBI. In some embodiments, the biomarkers of TBI comprise ubiquitin carboxy-terminal hydrolase L1 (UCH-L1), glial fibrillary acidic protein (GFAP), or a combination thereof. These methods involve detecting levels of the one or more biomarkers of TBI in one or more samples taken from the subject (e.g., a human subject) after an actual or suspected injury to the head. In some embodiments, the one or more samples are taken at a time point within 48 hours of an actual or suspected injury to the head. In some embodiments, detecting the levels of the one or more biomarkers of TBI comprises performing at least one assay in a sample obtained from a subject after an actual or suspected injury to the head. In some embodiments, the assay comprises contacting a sample, cither simultaneously or sequentially, in any order, with: (1) a capture antibody which binds to an epitope on the TBI biomarker to form a capture antibody-biomarker-complex, (2) a detection antibody which binds to an epitope on the TBI biomarker that is not bound by the capture antibody to form a capture antibody-biomarker-detection antibody complex, and (3) a solution comprising at least one nuclease.

In some embodiments, the present disclosure relates to improved methods for determining whether a subject's levels of one or more biomarkers, such as, biomarkers of TBI (e.g., GFAP, UCH-L1, or GFAP and UCH-L1) are elevated. In some embodiments, the methods comprise determining whether the subject's levels of the one or more biomarkers of TBI (GFAP, UCH-L1, or GFAP and UCH-L1) are elevated using an assay described above, and communicating the determination on or from at least one instrument. In some embodiments, determining whether the levels of the one or more biomarkers of TBI are elevated comprises performing at least one assay in a sample obtained from a subject after an actual or suspected injury to the head. In some embodiments, the assay comprises contacting a sample, either simultaneously or sequentially, in any order, with: (1) a capture antibody which binds to an epitope on the TBI biomarker to form a capture antibody-biomarker-complex, (2) a detection antibody which binds to an epitope on the TBI biomarker that is not bound by the capture antibody to form a capture antibody-biomarker-detection antibody complex, and (3) a solution comprising at least one nuclease.

In some embodiments, the methods involve detecting levels the one or more biomarkers of TBI in a sample obtained within about 48 hours after an actual or suspected injury to the head. For example, the methods may comprise detecting levels of GFAP, UCH-L1, or GFAP and UCH-L1 in a sample obtained within about 48 hours, within about 24 hours, or within about 12 hours after an actual or suspected injury to the head. In some embodiments, the methods comprise detecting levels of GFAP, UCH-L1, or GFAP and UCH-L1 in a sample obtained within about 12 hours to about 48 hours or within about 24 hours to about 48 hours. In yet still other embodiments, the methods comprise detecting levels of GFAP, UCH-L1, or GFAP and UCH-L1 within about 12 hours (e.g. within about 12 hours, within about 11 hours, within about 10 hours, within about 9 hours, within about 8 hours, within about 7 hours, within about 6 hours, within about 5 hours, within about 4 hours, within about 3 hours, within about 2 hours, within about 1 hour, or within about 30 minutes) after an actual or suspected injury to the head.

The present disclosure also relates to methods and systems that aid in the determination of whether a subject (e.g., a human subject) that has or may have sustained such an injury to the head would benefit from and thus receive a head computerized tomography (CT) scan, magnetic resonance imaging (MRI) procedure or both a head CT scan and a MRI procedure, based on the levels of one or more biomarkers such as UCH-L1, GFAP, or combination thereof. These methods involve detecting levels of at least one biomarker, such as UCH-L1, GFAP, or combination thereof, in one or more samples taken from the subject (e.g., a human subject) at a time point after an actual or suspected injury to the head. In some embodiments, the sample is taken from the subject within about 48 hours of an injury to the head (e.g., an actual injury) or suspected injury to the head. In some embodiments, levels of the at least one TBI biomarker are detected using an assay comprising contacting the sample, either simultaneously or sequentially, in any order, with: (1) a capture antibody which binds to an epitope on the TBI biomarker to form a capture antibody-biomarker-complex, (2) a detection antibody which binds to an epitope on the TBI biomarker that is not bound by the capture antibody to form a capture antibody-biomarker-detection antibody complex, and (3) a solution comprising at least one nuclease. For example, the methods may involve detecting levels of GFAP, UCH-L1, or UCH-L1 and GFAP within about 48 hours, within about 24 hours, or within about 12 hours after an actual or suspected injury to the head. The detection levels of the biomarker, such as UCH-L1, GFAP, or combination thereof, that are higher than reference levels of the biomarker after injury (e.g., an actual injury) or suspected injury to the head provides an aid in the determination of whether a subject should receive a head CT scan and/or a MRI procedure. For example, subjects (e.g., human subjects) having a level of the biomarker, such as UCH-L1, GFAP, or combination thereof, higher than a reference level of the biomarker, such as UCH-L1, GFAP, or a combination thereof, may also be identified as likely to have a positive head CT scan and/or MRI and thus benefit from having a CT scan and/or MRI procedure. Alternatively, subjects (e.g. human subjects) having a level of the biomarker, such as UCH-L1, GFAP, or combination thereof, lower than a reference level of the biomarker, such as UCH-L1, GFAP, or a combination thereof, may be identified as likely to have a negative head CT scan and/or MRI and thus would likely not benefit from a CT scan and/or MRI procedure.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Acquired brain injury” or (ABI) as used herein refers to damage to the brain that is caused by events occurring after birth. In other words, acquired brain injuries are not genetic or congenital but are the result neurological conditions and injuries. Acquired brain injuries are often divided into two categories. The first category is acquired traumatic brain injuries (TBI), that occur due to an external force, such as, for example, a sports injury, fall, physical shaking, blunt force trauma, explosion, blast, or exposure to a fire. The second category is non-traumatic acquired brain injuries that in some cases are caused by internal factors and include stroke, tumors, anoxia, infections, metabolic disorders, and others. As used herein, an acquired brain injury does not include or encompass damage to the brain that is caused by a stroke (including, such as, for example, ischemic stroke, hemorrhagic stroke, or a transient ischemic attack, etc.).

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al., J. Mol. Biol., 226:889-896 (1992). Selective mutation at selective mutagenesis positions and at contact or hypermutation positions with an activity-enhancing amino acid residue is described in U.S. Pat. No. 6,914,128 B1.

An “analog assay” as used herein refers to an assay in which the presence of and/or concentration of an analyte in a test sample is determined by measuring the total signal produced (e.g., fluorescence, color, etc.) by the analyte in an entire reaction mixture (e.g., a single reaction vessel). In an analog assay, the noise is indistinguishable from the signal. An example of an analog assay is an assay in which the presence of and/or concentration of an analyte is determined by measuring the total signal produced from a plurality of beads or microparticles contained in a single reaction vessel.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-UCH-L1 antibody or a UCH-L1 antibody).

An antibody as used herein also refers non-Ig derived alternatives (so-called antibody ‘mimetics’) such as, e.g., aptamers, DARPins, Affimers, Avimers, Knottins, Monobodies, and Affinity Clamps. As used herein, the term “aptamer” refers to a nucleic acid that has a specific binding affinity for a target analyte or molecule. It is recognized that affinity interactions are a matter of degree; however, in this context, the “specific binding affinity” of an aptamer for its analyte or target means that the aptamer binds to its analyte or target generally with a much higher degree of affinity than it binds to other components in a sample. An “aptamer” is a set of copies of one type or species of nucleic acid molecule that comprises a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single-stranded, double-stranded, or contain double-stranded regions, and can include higher ordered structures.

An aptamer can also be a photoaptamer, where a photoreactive or chemically reactive functional group is included in the aptamer to allow it to be covalently linked to its corresponding analyte or target. In some embodiments, an aptamer may include a detectable label. If an aptamer includes a detectable label, all copies of the aptamer need not have the same detectable label. Moreover, if different aptamers each include a detectable label, these different aptamers can have either the same detectable label or a different detectable label.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Aptamer” as used herein refers to a nucleic acid that has a specific binding affinity for a target analyte or molecule. It is recognized that affinity interactions are a matter of degree; however, in this context, the “specific binding affinity” of an aptamer for its analyte or target means that the aptamer binds to its analyte or target generally with a much higher degree of affinity than it binds to other components in a biological sample. An “aptamer” is a set of copies of one type or species of nucleic acid molecule that comprises a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single-stranded, double-stranded, or contain double-stranded regions, and can include higher ordered structures. An aptamer can also be a photoaptamer, where a photoreactive or chemically reactive functional group is included in the aptamer to allow it to be covalently linked to its corresponding analyte or target. In some aspects, an aptamer may include a detectable label.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

“Bead” and “particle” are used herein interchangeably and refer to a substantially spherical solid support. One example of a bead or particle is a microparticle. Microparticles that can be used herein can be any type known in the art. For example, the bead or particle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The microparticles can be of any size that would work in the methods described herein, e.g., from about 0.75 to about 5 nm, or from about 1 to about 5 nm, or from about 1 to about 3 nm. In other embodiments, the bead or particle can be a non-magnetic bead or particle, such as for example, non-magnetic polystyrene particles.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Biomarker”, “biomarker of interest” “analyte” or “analyte of interest”, as used interchangeably herein, refers to a naturally occurring or synthetic biological molecule found in an organism, in which the varying concentrations provide information useful in predicting the risk, occurrence, or severity of a disease, disorder, state, phase, or condition, such as traumatic brain injury (TBI) or other related disease phenotypes. In some embodiments, the biomarker is a biomolecule. Non-limiting examples of biomolecules include macromolecules such as proteins (e.g., peptides, polypeptides, protein fragments, protein complexes, fusion proteins, recombinant proteins, phosphoproteins, glycoproteins, lipoproteins). In some embodiments the biomarker or analyte is GFAP, UCH-L1, or a combination of GFAP and UCH-L1.

In some aspects, the biomarker may be a post-translationally modified protein (e.g., phosphorylated, methylated, glycosylated protein) and the first or the second binding member may be an antibody specific to a post-translational modification. A modified protein may be bound to a first binding member immobilized on a solid support where the first binding member binds to the modified protein but not the unmodified protein. In other embodiments, the first binding member may bind to both the unmodified and the modified protein, and the second binding member may be specific to the post-translationally modified protein.

In some aspects, the biomarker may be an aptamer. Aptamers, and use and methods of production thereof are reviewed in e.g., Shum et al., J Cancer Ther. 2013 4:872; Zhang et al., Curr Med Chem. 2011; 18:4185; Zhu et al., Chem Commun (Camb). 2012 48:10472; Crawford et al., Brief Funct Genomic Proteomic. 2003 2:72; Reverdatto et al., PLoS One 2013 8:065180.

“Bispecific antibody” is used herein to refer to a full-length antibody that is generated by quadroma technology (see Milstein et al., Nature, 305 (5934): 537-540 (1983)), by chemical conjugation of two different monoclonal antibodies (see, Staerz et al., Nature, 314 (6012): 628-631 (1985)), or by knob-into-hole or similar approaches, which introduce mutations in the Fc region (see Holliger et al., Proc. Natl. Acad. Sci. USA, 90 (14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within about an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Communicated” or “communicating” as used herein refers to the conveying, transmitting and/or reporting of an item of information. In some embodiments, the information that is communicated is an item of information obtained by performing an assay, such as, the amount or presence of a biomarker in a sample (e.g., a result). The information obtained by performing an assay can be communicated by a computer, in a document and/or spreadsheet, on a mobile device (e.g., a smart phone), on a website, in an e-mail, or any combination thereof. In some other embodiments, information is communicated on or from an instrument or device. In other embodiments, the information is communicated by being displayed, such as on an instrument or device.

A “clinically-relevant time frame” refers to a time frame (e.g., seconds, minutes, or hours) during which a careful and prudent medical practitioner (e.g., doctor, nurse, paramedic, or other) would reasonably consider the results of one or more biomarker tests to have a bearing on an imaging procedure, such as a head CT scan, or pursuant to guidelines established by an overseeing entity (e.g., a standard-setting body such as the World Health Organization (WHO), physicians review board, regulatory approval authority such as FEDA<EMEA, or other, etc.).

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pre-treatment reagent/solution, a substrate (e.g., as a solution), a stop solution, and the like that can be included in a kit or cartridge for assay of a test sample, such as a patient urine, whole blood, serum or plasma sample, in accordance with the methods described herein and other methods known in the art. Some components can be in solution or lyophilized for reconstitution for use in an assay.

“Correlated to” as used herein refers to compared to.

“CT scan” as used herein refers to a computerized tomography (CT) scan. A CT scan combines a series of X-ray images taken from different angles and uses computer processing to create cross-sectional images, or slices, of the bones, blood vessels and soft tissues inside your body. The CT scan may use X-ray CT, positron emission tomography (PET), single-photon emission computed tomography (SPECT), computed axial tomography (CAT scan), or computer aided tomography. The CT scan may be a conventional CT scan or a spiral/helical CT scan. In a conventional CT scan, the scan is taken slice by slice and after each slice the scan stops and moves down to the next slice, e.g., from the top of the abdomen down to the pelvis. The conventional CT scan requires patients to hold their breath to avoid movement artefact. The spiral/helical CT scan is a continuous scan which is taken in a spiral fashion and is a much quicker process where the scanned images are contiguous.

A head CT scan is “negative” for a TBI when no intracranial lesion(s) is observed in an image taken from a subject that has sustained, may have sustained or is suspected of sustaining an injury to the head. To further clarify, the head CT scan of a subject is “negative” for a TBI when a lesion is not found or identified; however, in some embodiments, the subject may still be experiencing symptoms (e.g., of TBI) even though the head CT is negative. Most subjects will be negative for a TBI on head CT given that not all injuries or lesions can be visualized by head CT. Consequently, the methods and assays described herein can be used to provide an assessment or determination of a subject with a negative head CT that may still have a TBI.

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. For example, a set of training samples comprising samples obtained from subjects known to have sustained an injury to the head (e.g., samples obtained from human subjects) known to have sustained a (i) mild TBI; and/or (ii) moderate, severe, or moderate to severe TBI and samples obtained from subjects (e.g., human subjects) known not to have sustained an injury to the head may be used to obtain assay-specific reference levels. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the disclosure. It is well within the ordinary skill of one in the art to determine the sensitivity and specificity associated with a given reference level in the methods of the disclosure, for example by repeated statistical analysis of assay data using a plurality of different possible reference levels.

Practically, when discriminating between a subject as having a traumatic brain injury or not having a traumatic brain injury or a subject as having a a mild versus a moderate, severe, or moderate to severe traumatic brain injury, the skilled person will balance the effect of raising a cutoff on sensitivity and specificity. Raising or lowering a cutoff will have a well-defined and predictable impact on sensitivity and specificity, and other standard statistical measures. It is well known that raising a cutoff will improve specificity but is likely to worsen sensitivity (proportion of those with disease who test positive). In contrast, lowering a cutoff will improve sensitivity but will worsen specificity (proportion of those without disease who test negative). The ramifications for detecting traumatic brain injury or determining a mild versus moderate, severe, or moderate to severe traumatic brain injury will be readily apparent to those skilled in the art. In discriminating whether a subject has or does not have a traumatic brain injury or a mild versus a moderate, severe, or moderate to severe traumatic brain injury, the higher the cutoff, specificity improves as more true negatives (i.e., subjects not having a traumatic brain injury, not having a mild traumatic brain injury, not have a moderate traumatic brain injury, not having a severe traumatic brain injury or not having a moderate to severe traumatic brain injury) are distinguished from those having a traumatic brain injury, a mild traumatic brain injury, a moderate traumatic brain injury, a severe traumatic brain injury or a moderate to severe traumatic brain injury. But at the same time, raising the cutoff decreases the number of cases identified as positive overall, as well as the number of true positives, so the sensitivity must decrease. Conversely, the lower the cutoff, sensitivity improves as more true positives (i.e., subjects having a traumatic brain injury, having a mild traumatic brain injury, having a moderate traumatic brain injury, having a severe traumatic brain injury or having a moderate to severe traumatic brain injury) are distinguished from those who do not have a traumatic brain injury, a mild traumatic brain injury, a moderate traumatic brain injury, a severe traumatic brain injury or a moderate to severe traumatic brain injury. But at the same time, lowering the cutoff increases the number of cases identified as positive overall, as well as the number of false positives, so the specificity must decrease.

Generally, a high sensitivity value helps one of skill rule out disease or condition (such as a traumatic brain injury, mild traumatic brain injury, moderate traumatic brain injury, severe traumatic brain injury or moderate to severe traumatic brain injury), and a high specificity value helps one of skill rule in disease or condition. Whether one of skill desires to rule out or rule in disease depends on what the consequences are for the patient for each type of error. Accordingly, one cannot know or predict the precise balancing employed to derive a test cutoff without full disclosure of the underlying information on how the value was selected. The balancing of sensitivity against specificity and other factors will differ on a case-by-case basis. This is why it is sometimes preferable to provide alternate cutoff (e.g., reference) values so a physician or practitioner can choose.

“Derivative” of an antibody as used herein may refer to an antibody having one or more modifications to its amino acid sequence when compared to a genuine or parent antibody and exhibits a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Digital assay” as used herein refers to an assay in which an analyte is captured and a molecule of the analyte segregated and interrogated (e.g., to detect the presence and/or concentration of the analyte in a sample). In a digital assay, noise is separated from signal. In a digital assay, the results are assigned a value of 1 or 0. Examples of digital assays include one or more of the following (which may overlap but are not mutually exclusive): single molecule detection assay, a nanowell assay, a single molecule enzyme linked immunosorbent assay, a direct capture counting assay, etc.

“Drugs of abuse” is used herein to refer to one or more additive substances (such as a drug) taken for non-medical reasons (such as for, example, recreational and/or mind-altering effects). Excessive overindulgence, use or dependence of such drugs of abuse is often referred to as “substance abuse”. Examples of drugs of abuse include alcohol, barbiturates, benzodiazepines, cannabis, cocaine, hallucinogens (such as ketamine, mescaline (peyote), PCP, psilocybin, DMT and/or LSD), methaqualone, opioids, amphetamines (including methamphetamines), anabolic steroids, inhalants (namely, substances which contain volatile substances that contain psychoactive properties such as, for example, nitrites, spray paints, cleaning fluids, markers, glues, etc.) and combinations thereof.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25:1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1) n-VD2-C-(X2) n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of UCH-L1, GFAP, or UCH-L1 and GFAP. Non-limiting examples of a DVD-Ig binding protein include (1) a DVD-Ig binding protein that binds one or more epitopes of UCH-L1, a DVD-Ig binding protein that binds an epitope of a human UCH-L1 and an epitope of UCH-L1 of another species (for example, mouse), and a DVD-Ig binding protein that binds an epitope of a human UCH-L1 and an epitope of another target molecule; (2) a DVD-Ig binding protein that binds one or more epitopes of GFAP, a DVD-Ig binding protein that binds an epitope of a human GFAP and an epitope of GFAP of another species (for example, mouse), and a DVD-Ig binding protein that binds an epitope of a human GFAP and an epitope of another target molecule; or (3) a DVD-Ig binding protein that binds one or more epitopes of UCH-L1 and GFAP, a DVD-Ig binding protein that binds an epitope of a human UCH-L1, a human GFAP, and an epitope of UCH-L1 of another species (for example, mouse), and a DVD-Ig binding protein that binds an epitope of a human UCH-L1, a human GFAP, and an epitope of another target molecule.

“Dynamic range” as used herein, refers to range over which an assay readout is proportional to the amount of target molecule or analyte in the sample being analyzed.

“Endonuclease” as used herein, refers to an enzyme which cleaves the phosphodiester bond within a polynucleotide chain. Endonucleases separate nucleotides other than the two nucleotides at the end (e.g., the 5′ end of the 3′ end) of the polynucleotide chain.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Fragment antigen-binding fragment” or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F(ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoids unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“GFAP” is used herein to describe glial fibrillary acidic protein. GFAP is a protein that is encoded by the GFAP gene in humans and by GFAP gene counterparts in other species, and which can be produced (e.g., by recombinant means, in other species).

“GFAP status” can mean either the level or amount of GFAP at a point in time (such as with a single measure of GFAP), the level or amount of GFAP associated with monitoring (such as with a repeat test on a subject to identify an increase or decrease in GFAP amount), the level or amount of GFAP associated with treatment for traumatic brain injury (whether a primary brain injury and/or a secondary brain injury), acquired brain injury, or other diseases, disorders or conditions, or combinations thereof.

“Glasgow Coma Scale” or “GCS” as used herein refers to a 15 point scale (e.g., described in 1974 by Graham Teasdale and Bryan Jennett, Lancet 1974; 2:81-4) that provides a practical method for assessing impairment of conscious level in patients who have suffered a brain injury. The test measures the best motor response, verbal response and eye opening response with these values: I. Best Motor Response (6—obey 2-part request; 5—brings hand above clavicle to stimulus on head neck; 4—bends arm at elbow rapidly but features not predominantly abnormal; 3—bends arm at elbow, features clearly predominantly abnormal; 2—extends arm at elbow; 1—no movement in arms/legs, no interfering factor; NT-paralyzed or other limiting factor); II. Verbal Response (5—correctly gives name, place and date; 4—not orientated but communication coherently; 3—intelligible single words; 2—only moans/groans; 1—no audible response, no interfering factor; NT-factor interfering with communication); and III. Eye Opening (4—open before stimulus; 3—after spoken or shouted request; 2—after fingertip stimulus; 1—no opening at any time, no interfering factor; NT—closed by local factor). The final score is determined by adding the values of I+II+III. A subject is considered to have a mild TBI if the GCS score is 13-15. A subject is considered to have a moderate TBI if the GCS score is 9-12. A subject is considered to have a severe TBI if the GCS score is 8 or less, typically 3-8.

“Glasgow Outcome Scale” as used herein refers to a global scale for functional outcome that rates patient status into one of five categories: Dead, Vegetative State, Severe Disability, Moderate Disability or Good Recovery. “Extended Glasgow Outcome Scale” or “GOSE” as used interchangeably herein provides more detailed categorization into eight categories by subdividing the categories of severe disability, moderate disability and good recovery into a lower and upper category as shown in Table 1.

TABLE 1

| | | | |
|---|---|---|---|
| 1 | Death | D | |
| 2 | Vegetative | VX | Condition of unawareness with only reflex |
| | state | | responses but with periods of spontaneous |
| | | | eye opening |
| 3 | Lower severe | SD− | Patient who is dependent for daily support |
| | disability | | for mental or physical disability, usually |
| 4 | Upper severe | SD+ | a combination of both. If the patient can |
| | disability | | be left alone for more than 8 hours at |
| | | | home it is upper level of SD, if not then |
| | | | it is low level of SD. |
| 5 | Lower moderate | MD− | Patients have some disability such as |
| | disability | | aphasia, hemiparesis or epilepsy and/or |
| 6 | Upper moderate | MD+ | deficits of memory or personality but are |
| | disability | | able to look after themselves. They are |
| | | | independent at home but dependent |
| | | | outside. If they are able to return to |
| | | | work even with special arrangement it is |
| | | | upper level of MD, if not then it is low |
| | | | level of MD. |
| 7 | Lower good | GR− | Resumption of normal life with the |
| | recovery | | capacity to work even if pre-injury status |
| 8 | Upper good | GR+ | has not been achieved. Some patients have |
| | recovery | | minor neurological or psychological |
| | | | deficits. If these deficits are not disabling |
| | | | then it is upper level of GR, if disabling |
| | | | then it is lower level of GR. |

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Injury to the head” or “head injury” as used interchangeably herein, refers to any trauma to the scalp, skull, or brain. Such injuries may include only a minor bump on the head or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. Alternatively, an injury to the head may be caused by the ingestion and/or exposure to a fire, chemical, toxin or a combination of a chemical and toxin. Examples of such chemicals and/or toxins include molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. Alternatively, an injury to the head may be caused as a result of a subject suffering from an autoimmune disease, a metabolic disorder, a brain tumor, hypoxia, a viral infection (e.g., SARS-CoV-2), a fungal infection, a bacterial infection, meningitis, hydrocephalus, or any combinations thereof. In some cases, it is not possible to be certain whether any such event or injury has occurred or taken place. For example, there may be no history on a patient or subject, the subject may be unable to speak, the subject may be aware of what events they were exposed to, etc. Such circumstances are described herein as the subject “may have sustained an injury to the head,” or as a “suspected head injury”. In certain embodiments herein, the closed head injury does not include and specifically excludes a cerebral vascular accident, such as stroke.

“Isolated polynucleotide” as used herein may mean a polynucleotide (e.g., of genomic, cDNA, or synthetic origin, or a combination thereof) that, by virtue of its origin, the isolated polynucleotide is not associated with all or a portion of a polynucleotide with which the “isolated polynucleotide” is found in nature; is operably linked to a polynucleotide that it is not linked to in nature; or does not occur in nature as part of a larger sequence.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111 In, 1251, 1311, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5 (6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16:1324-1328 (2006); Adamczyk et al., Bioorg. Med. Chem. Lett. 4:2313-2317 (2004); Adamczyk et al., Biorg. Med. Chem. Lett. 14:3917-3921 (2004); and Adamczyk et al., Org. Lett. 5:3779-3782 (2003)).

In one embodiment, the acridinium compound is an acridinium-9-carboxamide. Methods for preparing acridinium 9-carboxamides are described in Mattingly, J. Biolumin. Chemilumin. 6:107-114 (1991); Adamczyk et al., J. Org. Chem. 63:5636-5639 (1998); Adamczyk et al., Tetrahedron 55:10899-10914 (1999); Adamczyk et al., Org. Lett. 1:779-781 (1999); Adamczyk et al., Bioconjugate Chem. 11:714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5:3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl) acridinium fluorosulfonate (available from Cayman Chemical, Ann Arbor, MI). Methods for preparing acridinium 9-carboxylate aryl esters are described in McCapra et al., Photochem. Photobiol., 4:1111-21 (1965); Razavi et al., Luminescence 15:245-249 (2000); Razavi et al., Luminescence 15:239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6×His tag, which has an amino acid sequence of HHHHHH (SEQ ID NO: 3), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO:4) and derivatives thereof (e.g., ADDDDK (SEQ ID NO:5), etc.; (iii) Miscellaneous sequences can be used to link or connect the light and/or heavy chain variable regions of single chain variable region fragments. Examples of other linking sequences can be found in Bird et al., Science 242:423-426 (1988); Huston et al., PNAS USA 85:5879-5883 (1988); and McCafferty et al., Nature 348:552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen (e.g., although cross-reactivity or shared reactivity may occur). Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Magnetic resonance imaging” or “MRI” as used interchangeably herein refers to a medical imaging technique used in radiology to form pictures of the anatomy and the physiological processes of the body in both health and disease (e.g., referred to herein interchangeably as “an MRI”, “an MRI procedure” or “an MRI scan”). MRI is a form of medical imaging that measures the response of the atomic nuclei of body tissues to high-frequency radio waves when placed in a strong magnetic field, and that produces images of the internal organs. MRI scanners, which is based on the science of nuclear magnetic resonance (NMR), use strong magnetic fields, radio waves, and field gradients to generate images of the inside of the body.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Negative predictive value” or “NPV” as used interchangeably herein refers to the probability that a subject has a negative outcome given that they have a negative test result.

“Non-point-of-care device” refers to a device that is not a point-of-care device or a single use device. A non-point-of-care device refers to any device that does not meet any of the above limitations of a point-of-care or a single use device as defined herein. In some embodiments, the non-point-of-care device may be a relatively large instrument, such as a tabletop instrument, or even larger, such as a high-throughput instrument. Accordingly, in some embodiments the non-point-of-care device is not a handheld instrument. In some embodiments, the non-point-of-care device is capable of performing an assay on more than one clinical sample simultaneously. Suitable non-point-of-care devices include, for example, the ARCHITECT or Alinity platforms produced by Abbott Laboratories.

“Orthopedic injury” refers to one or more injuries to one or more parts of the musculoskeletal system, including injury to bones of the skeleton, muscles, cartilage, tendon, ligaments, joints, and other connective tissue that supports and binds tissues and organs together. In one embodiment, an orthopedic injury may be the result of a sudden accident and require medical attention. Examples of orthopedic injuries include dislocations (such as, for example, to a joint), fractures (including for example, stress or compression fractures) or breaks (such as, for example, to one or more bones), sprains (such as, for example, to an ankle, wrist, knee, shoulder, etc.), tears (such as, for example, a ligament tear such as ACL tear or meniscus tear, a cartilage tear such as a labral tear or a tendon and/or muscle tear such as a rotator cuff tear), or over use injuries (such as, for example, plantar fasciitis, tennis elbow, carpal tunnel syndrome). In one embodiment, the orthopedic injury is a fracture. In another embodiment, the orthopedic injury is a break. In another embodiment, the orthopedic injury is a sprain. In yet another embodiment, the orthopedic injury is a tear. In still another embodiment, the orthopedic injury is one or more of a fracture, break, sprain or tear.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, IL) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA). In some embodiments, the point-of-care device is a single-use device. The term “single-use device” or “single-use instrument” refers to a clinical diagnostic instrument that processes and performs a clinical diagnostic assay on a unit use basis (such as, for example, a single-use cartridge) for a single patient sample. A point-of-care instrument does not perform an assay on more than one clinical sample simultaneously. However, the point-of-care instrument may have the capability to measure more than one parameter (e.g., more than one analyte) in an individual clinical sample per unit use basis.

“Positive predictive value” or “PPV” as used interchangeably herein refers to the probability that a subject has a positive outcome given that they have a positive test result.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

A “receiver operating characteristic” curve or “ROC” curve refers to a graphical plot that illustrates the performance of a binary classifier system as its discrimination threshold is varied. For example, a ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). This disclosure provides exemplary reference levels. However, it is well-known that reference levels may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays. In certain embodiments described herein, the reference level is described as being determined by any assay having a certain specificity and sensitivity.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” “biological sample,” and “patient sample” as used interchangeably herein may be a sample of blood, such as whole blood (including for example, capillary blood, venous blood, dried blood spot, etc.), serum or plasma, or tissue, saliva, urine, amniotic fluid, an oropharyngeal specimen, a nasopharyngeal specimens, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Additionally, the sample can be a nasopharyngeal or oropharyngeal sample obtained using one or more swabs that, once obtained, is placed in a sterile tube containing a virus transport media (VTM) or universal transport media (UTM), and retained therein or transferred to another media for testing.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, frozen sections taken for histologic purposes, blood (such as whole blood, dried blood spots, etc.), plasma, serum, saliva, red blood cells, platelets, interstitial fluid, cerebral spinal fluid, etc. Cell types and tissues may also include lymph fluid, cerebrospinal fluid, or any fluid collected by aspiration. A tissue or cell type may be provided by removing a sample of cells from a human and a non-human animal but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some embodiments, the sample is a blood sample (e.g., a whole blood sample, a serum sample, or a plasma sample). In some embodiments, the sample is a whole blood sample. In some embodiments, the sample is a capillary blood sample. In some embodiments, the sample is a dried blood spot. In some embodiments, the sample is a serum sample. In yet other embodiments, the sample is a plasma sample. In some embodiments, the sample is an oropharyngeal specimen. In other embodiments, the sample is a nasopharyngeal specimen. In other embodiments, the sample is sputum. In other embodiments, the sample is endotracheal aspirate. In still yet other embodiments, the sample is bronchoalveolar lavage. In still yet other embodiments, the sample is a saliva sample.

“Sensitivity” of an assay as used herein refers to the proportion of subjects for whom the outcome is positive that are correctly identified as positive (e.g., correctly identifying those subjects with a disease or medical condition for which they are being tested). For example, this might include correctly identifying subjects as having a TBI as distinct from those who do not have a TBI, correctly identifying subjects having a moderate, severe, or moderate to severe TBI as distinct from those having a mild TBI, correctly identifying subjects as having a mild TBI as distinct from those having a moderate, severe, or moderate to severe TBI, correctly identifying subjects as having a moderate, severe, or moderate to severe TBI as distinct from those having no TBI or correctly identifying subjects as having a mild TBI as distinct from those having no TBI etc.

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects not having an TBI as distinct from those who do have a TBI, correctly identifying subjects not having a moderate, severe, or moderate to severe TBI as distinct from those having a mild TBI, or correctly identifying subjects as not having a mild TBI as distinct from those having a moderate, severe, or moderate to severe TBI, etc.)

“Series of calibrating compositions” refers to a plurality of compositions comprising a known concentration of (1) UCH-L1, wherein each of the compositions differs from the other compositions in the series by the concentration of UCH-L1; and/or (2) GFAP, wherein each composition differs from the other compositions in the series by the concentration of GFAP.

“Solid phase” or “solid support” as used interchangeably herein, refers to any material that can be used to attach and/or attract and immobilize (1) one or more capture reagents or capture specific binding partners, or (2) one or more detection reagents or detection specific binding partners. The solid phase can be chosen for its intrinsic ability to attract and immobilize a capture reagent. Alternatively, the solid phase can have affixed thereto a linking agent that has the ability to attract and immobilize the (1) capture reagent or capture specific binding partner, or (2) detection reagent or detection specific binding partner. For example, the linking agent can include a charged substance that is oppositely charged with respect to the capture reagent (e.g., capture specific binding partner) or detection reagent (e.g., detection specific binding partner) itself or to a charged substance conjugated to the (1) capture reagent or capture specific binding partner or (2) detection reagent or detection specific binding partner. In general, the linking agent can be any binding partner (preferably specific) that is immobilized on (attached to) the solid phase and that has the ability to immobilize the (1) capture reagent or capture specific binding partner, or (2) detection reagent or detection specific binding partner through a binding reaction. The linking agent enables the indirect binding of the capture reagent to a solid phase material before the performance of the assay or during the performance of the assay. For example, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art. In some embodiments, the solid phase is polystyrene or derivatized polystyrene.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced, and aptamers.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

“Sub-acute” as used herein refers to an injury (e.g., such as a traumatic brain injury) that is between an acute and chronic stage. In some embodiments, sub-acute refers to an injury from after about 24 hours to about three weeks after an actual or suspected injury to the head.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In some embodiments, the subject is a human. The subject or patient may be undergoing other forms of treatment.

“Treat,” “treating” or “treatment” are each used interchangeably herein to describe reversing, alleviating, ameliorating, or inhibiting the progress of a disease, disorder, state, phase and/or injury, or one or more symptoms of such disease, disorder, state, phase and/or injury to which such term applies. In some embodiments, a treatment may be either performed in an acute or chronic way. Depending on the condition of the subject, the term also refers to preventing a disease, disorder, state, phase and/or injury, and includes preventing the onset of a disease, disorder, state, phase and/or injury, or preventing the symptoms associated with a disease, disorder, state, phase and/or injury. “Preventing” also refers to preventing the recurrence of a disease, disorder, state, phase and/or injury or of one or more symptoms associated with such disease, disorder, state, phase and/or injury. “Treatment” and “therapeutically,” refer to the act of treating, as “treating” is defined above. In some aspects, the prevention or treatment of a disease, disorder, state, phase and/or injury can be done prior to affliction or injury with the disease, disorder, state, phase and/or injury, such as, for example, to reduce the severity of a disease, disorder, state, phase and/or injury or symptoms associated with a disease, disorder, state, phase and/or injury. Such treatment (including prevention or reduction) can include (a) administration of one or more pharmaceutical composition(s) and/or one or more nutritional composition(s) to a subject; (b) the use of one or more of physical therapy, occupational therapy, and/or counseling; (c) rest or reducing or refraining from normal daily activities, or certain activities (e.g., sports, work, exercise); or (d) any combinations of (a), (b) and (c).

In some embodiments, the prevention or treatment of a disease can be done prior to affliction or injury with the disease, disorder, state, phase and/or injury, such as, for example, to reduce the severity of a disease, disorder, state, phase and/or injury or symptoms associated with a disease, disorder, state, phase and/or injury. Such treatment (including prevention or reduction) can include (a) administration of one or more pharmaceutical composition and/or one or more nutritional compositions to a subject; (b) the use of one or more of physical therapy, occupational therapy, and/or counseling; (c) rest or reducing or refraining from normal daily activities, or certain activities (e.g., sports, work, exercise); or (d) any combinations of (a), (b) and (c).

“Traumatic Brain Injury” or “TBI” as used interchangeably herein refers to a complex injury with a broad spectrum of symptoms and disabilities. TBI is most often an acute event similar to other injuries. TBI can be classified as “mild,” “moderate,” “moderate to severe”, or “severe.” The causes of TBI are diverse and include, for example, physical shaking by a person, a car accident, injuries from firearms, cerebral vascular accidents (e.g., strokes), falls, explosions or blasts and other types of blunt force trauma. Other causes of TBI include the ingestion and/or exposure to one or more fires, chemicals or toxins (such as molds, asbestos, pesticides and insecticides, organic solvents, paints, glues, gases (such as carbon monoxide, hydrogen sulfide, and cyanide), organic metals (such as methyl mercury, tetraethyl lead and organic tin), one or more drugs of abuse or combinations thereof). Alternatively, TBI can occur in subjects suffering from an autoimmune disease, a metabolic disorder, a brain tumor, hypoxia, a viral infection (e.g., SARS-CoV-2, meningitis, etc., a fungal infection (e.g., meningitis), a bacterial infection (e.g., meningitis), hydrocephalus, or any combinations thereof. Young adults and the elderly are the age groups at highest risk for TBI. In certain embodiments herein, traumatic brain injury or TBI does not include and specifically excludes cerebral vascular accidents such as strokes.

“Mild TBI” as used herein refers to a head injury where a subject may or may not experience a loss of consciousness. For subjects that experience a loss of consciousness, it is typically brief, usually lasting only a few seconds or minutes. Mild TBI is also referred to as a concussion, minor head trauma, minor TBI, minor brain injury, and minor head injury. While MRI and CT scans are often normal, the individual with mild TBI may have cognitive problems such as headache, difficulty thinking, memory problems, attention deficits, mood swings and frustration.

Mild TBI is the most prevalent TBI and is often missed at time of initial injury. Typically, a subject has a Glasgow Coma Scale score of between 13-15 (such as 13-15 or 14-15). Fifteen percent (15%) of people with mild TBI have symptoms that last 3 months or more. Common symptoms of mild TBI include fatigue, headaches, visual disturbances, memory loss, poor attention/concentration, sleep disturbances, dizziness/loss of balance, irritability-emotional disturbances, feelings of depression, and seizures. Other symptoms associated with mild TBI include nausea, loss of smell, sensitivity to light and sounds, mood changes, getting lost or confused, and/or slowness in thinking.

“Moderate TBI” as used herein refers to a brain injury where loss of consciousness and/or confusion and disorientation is between 1 and 24 hours and the subject has a Glasgow Coma Scale score of between 9-13 (such as 9-12 or 9-13). The individual with moderate TBI may have abnormal brain imaging results. “Severe TBI” as used herein refers to a brain injury where loss of consciousness is more than 24 hours and memory loss after the injury or penetrating skull injury longer than 24 hours and the subject has a Glasgow Coma Scale score between 3-8. The deficits range from impairment of higher level cognitive functions to comatose states. Survivors may have limited function of arms or legs, abnormal speech or language, loss of thinking ability or emotional problems. Individuals with severe injuries can be left in long-term unresponsive states. For many people with severe TBI, long-term rehabilitation is often necessary to maximize function and independence.

“Moderate to severe” TBI as used herein refers to a spectrum of brain injury that includes a change from moderate to severe TBI over time and thus encompasses (e.g., temporally) moderate TBI alone, severe TBI alone, and moderate to severe TBI combined. For example, in some clinical situations, a subject may initially be diagnosed as having a moderate TBI but who, over the course of time (minutes, hours or days), progresses to having a severe TBI (such, as for example, in situations when there is a brain bleed). Alternatively, in some clinical situations, a subject may initially be diagnosed as having a severe TBI but who, over the course of time (minutes, hours or days), progresses to having a moderate TBI. Such subjects would be examples of patients that could be classified as “moderate to severe”. Common symptoms of moderate to severe TBI include cognitive deficits including difficulties with attention, concentration, distractibility, memory, speed of processing, confusion, perseveration, impulsiveness, language processing, and/or “executive functions”, not understanding the spoken word (receptive aphasia), difficulty speaking and being understood (expressive aphasia), slurred speech, speaking very fast or very slow, problems reading, problems writing, difficulties with interpretation of touch, temperature, movement, limb position and fine discrimination, the integration or patterning of sensory impressions into psychologically meaningful data, partial or total loss of vision, weakness of eye muscles and double vision (diplopia), blurred vision, problems judging distance, involuntary eye movements (nystagmus), intolerance of light (photophobia), hearing issues, such as decrease or loss of hearing, ringing in the cars (tinnitus), increased sensitivity to sounds, loss or diminished sense of smell (anosmia), loss or diminished sense of taste, the convulsions associated with epilepsy that can be several types and can involve disruption in consciousness, sensory perception, or motor movements, problems with control of bowel and bladder, sleep disorders, loss of stamina, appetite changes, problems with regulation of body temperature, menstrual difficulties, dependent behaviors, issues with emotional ability or stability, lack of motivation, irritability, aggression, depression, disinhibition, or denial/lack of awareness. Subjects having a moderate to severe TBI can have a Glasgow Coma Scale score from 3-12 (which includes the range of 9-12 for a moderate TBI, and 3-8 for a severe TBI).

“Ubiquitin carboxy-terminal hydrolase L1” or “UCH-L1” as used interchangeably herein refers to a deubiquitinating enzyme encoded by the UCH-L1 gene in humans and by UCH-L1 gene counterparts in other species. UCH-L1, also known as ubiquitin carboxyl-terminal esterase L1 and ubiquitin thiolesterase, is a member of a gene family whose products hydrolyze small C-terminal adducts of ubiquitin to generate the ubiquitin monomer.

“UCH-L1 status” can mean either the level or amount of UCH-L1 at a point in time (such as with a single measure of UCH-L1), the level or amount of UCH-L1 associated with monitoring (such as with a repeat test on a subject to identify an increase or decrease in UCH-L1 amount), the level or amount of UCH-L1 associated with treatment for traumatic brain injury (whether a primary brain injury and/or a secondary brain injury), acquired brain injury, or other diseases, disorders or conditions, or combinations thereof.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one embodiment, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-UCH-L1 antibody that differs from the corresponding fragment of anti-UCH-L1 antibody in amino acid sequence but is still antigenically reactive and can compete with the corresponding fragment of anti-UCH-L1 antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Cartridges for Use in Point-of-Care Devices

In some embodiments, provided herein are cartridges for use in point-of-care devices. The cartridges used in a point-of-care device comprise a housing. The housing contains at least a top portion and a bottom portion. Disposed within the housing is a biosensor chip, a sample chamber, a waste chamber, and all reagents necessary to perform an assay. The cartridge may also contain an air bladder, as well as fluid conduits, vents, and valve elements required to execute the assay. Generally, all fluid movements (such as of the sample and other reagents) are automatically controlled by the point-of-care device, such as by electromechanical or other interactions with the cartridge. In some further embodiments, the cartridge is a single-use cartridge such as that described in WO 2013/192289, the contents of which are herein incorporated by reference.

As mentioned, the housing comprises all the reagents necessary to perform an assay, such as, for example, pre-treatment reagents. Pre-treatment reagents can comprise: (a) one or more salts; (b) one or more anti-IgM antibodies; (c) one or more anti-IgG antibodies; (d) one or more nucleases; or (c) any combinations of (a), (b), (c) and (d). In some embodiments, the pre-treatment reagents can be printed as a layer (“pre-treatment printed layer”) in an appropriate location within the housing. For example, the pre-treatment printed layer can be printed in a location in the housing (e.g., such as in a sample chamber) to ensure that the sample is placed in contact with the pre-treatment printed layer prior to an assay being performed. In some embodiments, once the sample is in contact with the pre-treatment printed layer, the pre-treatment reagents contained in the pre-treatment printed layer dissolve into the sample.

In some embodiments, the one or more nucleases used in the pre-treatment reagents can be an endonuclease. In other embodiments, the one or more nucleases used in the pre-treatment reagents is a Benzonase® nuclease. Benzonase® nuclease is a Serratia marcescens nuclease (e.g., otherwise known as CAS Number: 9025-65-4, or Enzyme Commission number: 3.1.30.2 (BRENDA, IUBMB), or MDL number: MFCD00131010).

In some embodiments, the one or more salts used in the pre-treatment reagents is a chloride, such as magnesium chloride.

As mentioned previously, the cartridge comprises a biosensor chip. The biosensor chip contains one or more sensors comprising one or more reagents for performing an assay. Specifically, the chip can contain one or more “capture sensors”. The capture sensors comprise one or more antibodies or antigens that specifically bind to a biomarker or analyte of interest in a sample (“capture reagents”). The capture reagents are coated onto one or more solid supports (e.g., such as one or more microparticles) and immobilized (e.g., dried) on to the surface of the capture sensor.

The chip also comprises one or more areas containing one or more antibodies or antigens labeled with a detectable label that specifically binds to the biomarker or analyte of interest in the sample (“detection reagents”). In some embodiments, the detection reagents are printed or dried on the surface of the chip. Generally, the detection reagents are printed or dried upstream to the capture sensors. Optionally, the chip can further comprise one or more “reference sensors” comprising one or more solid supports immobilized (e.g., dried) on the sensor. The reference sensors are typically placed upstream to the capture sensors but downstream of the detection reagents. The reference sensors do not contain any capture or detection reagents but are present in the event the biomarker or analyte is not captured by the capture sensor. The reference sensors can be used to subtract signal that occurs due to the non-specific binding of reagents. This is useful not only in the absence of a biomarker or analyte of interest, but at low levels of the biomarker or analyte of interest as well, until the signal on the capture sensor is greater than what is measured on the reference signal.

To perform the assay, a fluid containing the sample and any pre-treatment reagents is exposed to the area containing the detection reagents and the capture sensors. Generally, the fluid containing the sample and any pre-treatment reagents flows over the area containing the detection reagents first and the detection reagents bind to any biomarker or analyte of interest in the sample to form biomarker/analyte-detection reagent complexes. The fluid containing the biomarker/analyte-detection reagent complexes moves continuously from area containing the detection reagents to the capture sensor where the capture reagents bind to the biomarker/analyte-detection reagent complexes to form biomarker/analyte reagent-analyte-detection reaction complexes which can be detected using routine techniques known in the art.

For example, in the context of the present disclosure, a cartridge may contain: (a) first capture sensor comprising immobilized anti-GFAP antibodies which are coated on to one or more solid supports (“GFAP capture sensor”); (b) a first reference sensor that is upstream to the first capture sensor; (c) a second capture sensor comprising immobilized anti-UCH-L1 antibodies which are coated on to one or more solid supports (“UCH-L1 capture sensor”); (d) a second reference sensor upstream to the second capture sensor; and (c) an area upstream of the first capture sensor, first reference sensor, second capture sensor, and second reference sensor, comprising printed or dried anti-UCH-L1 antibodies labeled with a first detectable label and anti-GFAP antibodies labeled with a second detectable label (where the first detectable label and the second detectable label can be the same label or different labels). To perform the assay, a fluid containing the sample and any pre-treatment reagents flows over the area containing the anti-UCH-L1 antibodies labeled with a first detectable label (“UCH-L1 detection reagent”) and the anti-GFAP antibodies labeled with a second detectable label (“GFAP detection reagent). The UCH-L1 detection reagent will bind to any UCH-L1 in the fluid to form a UCH-L1-UCH-L1 detection reagent complexes and/or the GFAP detection reagent will bind to any GFAP in the fluid to form a GFAP-GFAP detection reagent complexes. The UCH-L1-UCH-L1 detection reagent complexes and the GFAP-GFAP detection reagent complexes continuously flow to the GFAP capture sensor and the UCH-L1 capture sensor. The anti-GFAP antibodies immobilized on the GFAP capture sensor (“GFAP capture reagent”) will bind to the GFAP-GFAP detection reagent complexes to form GFAP capture reagent-GFAP-GFAP detection reagent complexes which can be detected using routine techniques known in the art. The anti-UCH-L1 antibodies immobilized on the UCH-L1 capture sensor (“UCH-L1 capture reagent”) will bind to the UCH-L1-UCH-L1 detection reagent complexes to form UCH-L1 capture reagent-UCH-L1-UCH-L1 detection reagent complexes which can be detected using routine techniques known in the art.

### 3. Methods of Measuring a Level of a Biomarker in a Biological Sample Obtained from a Subject

In some aspects, provided herein are methods for measuring an amount or level of a biomarker in biological sample obtained from a subject to assess, determine and/or diagnose whether a subject has suffered an injury and/or is suffering from a disease or other medical condition. In some aspects, the methods involve measuring a level of more than one biomarker. In other embodiments, the methods for measuring an amount or level of a biomarker in a sample comprise performing an assay on a sample obtained from the subject suspected of or who has suffered an injury or is suspected of or is suffering from a disease or other medical condition. In some embodiments, the assay comprises contacting a sample, either simultaneously or sequentially, in any order, with: (1) one or more capture antibodies which bind to an epitope on at least one biomarker to form a capture antibody-biomarker-complex, (2) one or more detection antibodies which bind to an epitope on the biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

In still yet other embodiments, the methods involve measuring a level of more than one biomarker. In some other embodiments, the methods for measuring an amount or concentration of a biomarker of in a sample comprise performing an assay on a sample obtained from a subject suspected of or who has suffered an injury or is suspected of or is suffering from a disease or other medical condition. In some embodiments, the assay comprises contacting a sample, either simultaneously or sequentially, in any order, with: (1) one or more capture antibodies which bind to an epitope on the biomarker to form a capture antibody-biomarker-complex, (2) one or more detection antibodies which bind to an epitope on the TBI biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

In some embodiments, the methods involve measuring an amount or level of more than one biomarker to assess, determine and/or diagnose whether a subject has suffered an injury and/or is suffering from a disease or other medical condition. In some other embodiments, the methods for measuring an amount or concentration of a biomarker in a sample comprise performing an assay on a sample obtained from the subject suspected of suffering (or who has suffered) from an injury, disease, or other medical condition. In some embodiments, the assay comprises contacting a sample, either simultaneously or sequentially, in any order, with: (1) one or more capture antibodies which bind to an epitope on the biomarker to form a capture antibody-biomarker-complex, (2) one or more detection antibodies which bind to an epitope on the biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

The use of a solution containing a nuclease in the methods described herein prevents or disrupts non-specific detection antibody (e.g., conjugate antibody) binding during the assay, particularly with the use of whole blood samples. Moreover, it has also been found that the use of a solution containing a nuclease in the methods described herein reduces the formation of aggregates (such as DNA aggregates) when compared to a method that does not use a solution containing a nuclease. While not wishing to be bound by any theory, when whole blood samples are used in certain assays such as described herein, the white blood cells (e.g., buffy coat) contained in the whole blood release DNA (e.g., nucleic acids) that aggregate with other molecules present during performance of the assay (e.g., heparin, salts, etc.) which causes non-specific detection antibody (e.g., conjugate antibody) binding. Additionally, in a cartridge used in a point-of-care device, nucleic acids from white blood cells in a whole blood sample are released within the cartridge. These nucleic acids polymerize within the cartridge thereby forming strands which adhere to capture and reference sensors through electrostatic interactions between the strands and any pre-treatment reagents). The presence of these nucleic acid strands which adhere to the capture and reference sensors can cause errors in the results reported by an assay. The use of a solution (e.g., such as a pre-treatment solution) containing a nuclease or to which a nuclease is added in the methods described herein may: (a) prevent or disrupt the formation of such aggregates, particularly when the sample is a whole blood sample; and (b) when used in a cartridge in a point-of-care device, prevent or inhibit the nucleic acid strands from adhering to one or more of the capture and/or reference sensor, thereby preventing errors from being reported by the assay.

In some embodiments the nuclease is an endonuclease or nuclease. In some embodiments, the nuclease cleaves DNA (e.g., is a DNase). In some embodiments, the nuclease cleaves RNA (e.g., is an RNase) but as described herein, preferably a nuclease that cleaves RNA (i.e., an RNase) also must have DNA cleavage activity (i.e., must also be a DNase). In some embodiments, the nuclease cleaves DNA and RNA. In some embodiments, the nuclease is a Benzonase® nuclease. Benzonase® nuclease is also known as a Serratia marcescens nuclease. In yet other embodiments, the solution comprising a nuclease comprises at least about 1 U/mL nuclease, such as, for example, a Benzonase® nuclease. In some embodiments, the solution comprising a nuclease comprises about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease. In still other embodiments, the final concentration or amount of nuclease used in the methods described herein is about 1.0 U/mL of nuclease. In still further embodiments, the final concentration or amount of Benzonase® nuclease used in the methods described herein is about 1.0 U/mL. In yet further embodiments, when included in a cartridge for use in a point-of-care device, the amount of nuclease (e.g., Benzonase® nuclease) included in a pre-treatment reagent is about 1.5 U/mL. Once the pre-treatment reagent containing a nuclease is mixed with the biological sample, the final concentration of nuclease in the sample is about 1.0 U/mL.

In some embodiments, the solution comprising a nuclease does not contain heparin. It has also been found that avoiding the use of heparin in the solution comprising the nuclease can further aid in preventing or disrupting the formation of aggregates that may otherwise occur during performance of the assay. In some embodiments, the method does not use any heparin.

In some embodiments, the solution comprising a nuclease further comprises one or more salts, such as magnesium chloride.

In some embodiments, the method comprises contacting the sample with the solution comprising a nuclease before contacting the sample with the capture antibody and/or the detection antibody. For example, the sample may be contacted with the solution comprising the nuclease as a pre-treatment step, or as part of a sample pre-treatment step. In yet other embodiments, the method comprises contacting the sample with the solution comprising a nuclease at the same time as contacting the sample with the capture antibody and/or detection antibody. In still yet other embodiments, the method comprises contacting the sample comprising a nuclease after contacting the sample with the capture antibody and/or detection antibody.

In some embodiments, the method comprises contacting the sample with the solution comprising the nuclease before contacting the sample with the capture antibody. In some embodiments, the method comprises contacting the sample with the solution comprising a nuclease after contacting the sample with the capture antibody (e.g., after formation of the capture antibody-biomarker complex). In some embodiments, the method comprises contacting the sample with the solution comprising a nuclease after contacting the sample with the capture antibody and the detection antibody (e.g., after formation of the capture antibody-biomarker-detection antibody complex). In still other embodiments, the method comprises contacting the sample with the solution comprising the nuclease at the same time the sample is contacted with the capture antibody and/or detection antibody. In still other embodiments, the method comprises contacting the sample with the solution comprising the nuclease after the sample is contacted with the capture antibody and/or detection antibody (e.g., after complex formation). In some embodiments, the method further comprises measuring the amount or concentration of the biomarker in the sample based upon the signal generated by the detectable label in the capture antibody-biomarker-detection antibody complex.

In some embodiments, the biomarker is GFAP. For example, determining the amount or level of GFAP from a biological sample of a subject can be used to assess and/or determine whether the subject has sustained an acquired brain injury, such as, an injury to the head (e.g., such as a traumatic brain injury), has suffered a stroke (such as an ischemic stroke), has SARS-CoV-2, has neuronal apotosis (e.g., such as that induced by deep hypothermic circulatory arrest), or has white matter lesions (subcortical), Parkinson's disease, Alzheimer's disease, Alexander disease, cancer (e.g., such as glioblastoma), or infection (such as Toxocara ova, lyme neuroborreliosis, etc.). Alternatively, determining the amount or level of GFAP can be used to assess, determine and/or diagnose whether a subject has sustained an injury to the head (e.g., such as a traumatic brain injury), has suffered a stroke (such as an ischemic stroke), an intracerebral hemorrhage, or astrocytic injury (such as that caused by SARS-CoV-2), or has Alzheimer's disease, Alexander disease, cancer (e.g., such as glioblastoma), or infection (such as Toxocara ova, lyme neuroborreliosis, etc.). Thus, in some embodiments the assay comprises contacting the sample, either simultaneously or sequentially, in any order, with: (1) one or more capture antibodies which bind to an epitope on GFAP to form a capture antibody-GFAP antigen-complex, (2) one or more detection antibodies which bind to an epitope on GFAP that is not bound by the capture antibody to form a capture antibody-GFAP antigen-detection antibody complex, and (3) a solution comprising a nuclease. Either the capture antibody or the detection antibody may comprise a detectable label. In some embodiments, the method comprises contacting the sample with the solution comprising a nuclease (e.g., an endonuclease) after contacting the sample with one or more capture antibodies (e.g., after formation of the capture antibody-GFAP antigen-complex). In some embodiments, the method comprises contacting the sample with the solution comprising a nuclease (e.g., an endonuclease) after contacting the sample with the one or more capture antibodies and the one or more detection antibodies (e.g., after formation of the capture antibody-GFAP antigen-detection antibody complex). In some embodiments, the method further comprises measuring the amount or concentration of GFAP in the sample based upon the signal generated by the detectable label in the capture antibody-GFAP antigen-detection antibody complex. In some embodiments, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In some embodiments, the solution comprising a nuclease does not contain heparin. In some embodiments, the method does not use any heparin.

In some embodiments, the biomarker is UCH-L1. For example, determining an amount or level of UCH-L1 in a biological sample obtained from a subject can be used to assess, determine and/or diagnose whether the subject has sustained an acquired brain injury, such as, an injury to the head (e.g., such as a traumatic brain injury), has suffered a stroke (such as an ischemic stroke), has SARS-CoV-2, has neuronal apotosis (e.g, such as that induced by deep hypothermic circulatory arrest), or has white matter lesions (subcortical), Parkinson's disease, Alzheimer's disease, Alexander disease, cancer (e.g., such as glioblastoma), or infection (such as Toxocara ova, lyme neuroborreliosis, etc.). Alternatively, determining the amount or level of UCH-L1 can be used to assess, determine and/or diagnose whether a subject has sustained an injury to the head (e.g., such as a traumatic brain injury), has suffered a stroke (such as an ischemic stroke), an intracerebral hemorrhage, or astrocytic injury (such as that caused by SARS-CoV-2), or has Alzheimer's disease, Alexander disease, cancer (e.g., such as glioblastoma), or infection (such as Toxocara ova, lyme neuroborreliosis, etc.). Thus, in some embodiments the assay comprises contacting the sample, either simultaneously or sequentially, in any order, with: (1) one or more capture antibodies which bind to an epitope on UCH-L1 to form a capture antibody-UCH-L1 antigen-complex, (2) one or more detection antibodies which bind to an epitope on UCH-L1 that is not bound by the capture antibody to form a capture antibody-UCH-L1 antigen-detection antibody complex, and (3) a solution comprising a nuclease. Either the capture antibody or the detection antibody may comprise a detectable label. In some embodiments, the method comprises contacting the sample with a solution comprising a nuclease (e.g., an endonuclease) before contacting the sample with the capture antibody. In some embodiments, the method comprises contacting the sample with the solution comprising a nuclease (e.g., an endonuclease) after contacting the sample with the capture antibody (e.g., after formation of the capture antibody-UCH-L1 antigen-complex). In still other embodiments, the method comprises contacting the sample with a solution comprising a nuclease (e.g., an endonuclease) at the same time as contacting the sample with the capture antibody and/or detection antibody. In some embodiments, the method comprises contacting the sample with the solution comprising a nuclease (e.g., an endonuclease) after contacting the sample with the capture antibody and the detection antibody (e.g., after formation of the capture antibody-UCH-L1 antigen-detection antibody complex). In some embodiments, the method further comprises measuring the amount or concentration of UCH-L1 in the sample based upon the signal generated by the detectable label in the capture antibody-UCH-L1 antigen-detection antibody complex. In some embodiments, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In some embodiments, the solution comprising a nuclease does not contain heparin.

### 4. Methods of Measuring a Level of a Biomarker of Acquired Brain Injury

In some embodiments, provided herein are methods for measuring an amount or level of a biomarker of acquired brain injury (ABI), such as, for example, traumatic brain injury (TBI) in a sample. In some embodiments, the methods involve measuring a level of more than one ABI biomarker. As used herein, a “biomarker of ABI” and “ABI” biomarker are used interchangeably. Additionally, as used herein, a “biomarker of TBI” and “TBI biomarker” are used interchangeably herein.

In some embodiments, the ABI biomarker is GFAP, UCH-L1, or GFAP and UCH-L1. In some other embodiments, the methods for measuring an amount, concentration or level of an ABI biomarker in a sample comprise performing an assay on a sample obtained from the subject after an actual or suspected injury to the head. In some embodiments, the assay comprises contacting a sample, cither simultaneously or sequentially, in any order, with: (1) one or more capture antibodies which bind to an epitope on the ABI biomarker to form a capture antibody-biomarker-complex, (2) one or more detection antibodies which bind to an epitope on the ABI biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

In still yet other embodiments, the methods involve measuring a level of more than one TBI biomarker. In some embodiments, the ABI biomarker is GFAP, UCH-L1, or GFAP and UCH-L1. In some other embodiments, the methods for measuring an amount, concentration, or level of a ABI biomarker in a sample comprise performing an assay on a sample obtained from the subject after an actual or suspected injury to the head. In some embodiments, the assay comprises contacting a sample, either simultaneously or sequentially, in any order, with: (1) one or more capture antibodies which bind to an epitope on the TBI biomarker to form a capture antibody-biomarker-complex, (2) one or more detection antibodies which bind to an epitope on the ABI biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

In some embodiments, the ABI biomarker is a TBI biomarker. In still other embodiments, the methods involve measuring a level of more than one TBI biomarker. In some embodiments, the TBI biomarker is GFAP, UCH-L1, or GFAP and UCH-L1. In some other embodiments, the methods for measuring an amount, concentration or level of a TBI biomarker in a sample comprise performing an assay on a sample obtained from the subject after an actual or suspected injury to the head. In some embodiments, the assay comprises contacting a sample, either simultaneously or sequentially, in any order, with: (1) one or more capture antibodies which bind to an epitope on the TBI biomarker to form a capture antibody-biomarker-complex, (2) one or more detection antibodies which bind to an epitope on the TBI biomarker that is not bound by the capture antibody to form a capture antibody-biomarker-detection antibody complex, and (3) a solution comprising a nuclease. For example, in some embodiments, the nuclease is added before the capture antibody and/or detection antibody are added (e.g., as a pre-treatment step). In other embodiments, the nuclease is added at the same time the capture antibody and/or detection antibody are added. In yet other embodiments, the nuclease is added after the capture antibody and/or detection antibody is added. Either the capture antibody or the detection antibody may comprise a detectable label. In some embodiments, the capture antibody comprises a detectable label. In some embodiments, the detection antibody comprises a detectable label.

The use of a solution containing a nuclease in the methods described herein prevents or disrupts non-specific detection antibody (e.g., conjugate antibody) binding during the assay, particularly with the use of whole blood samples. Moreover, it has also been found that the use of a solution containing a nuclease in the methods described herein reduces the formation of aggregates. While not wishing to be bound by any theory, when whole blood samples are used in certain assays, the white blood cells (e.g., buffy coat) contained in the whole blood release DNA (e.g., nucleic acids) that aggregate with other molecules present during performance of the assay (e.g., heparin, salts, etc.) which causes non-specific detection antibody (e.g., conjugate antibody) binding. Additionally, in a cartridge used in a point-of-care device, nucleic acids from white blood cells in a whole blood sample are released within the cartridge. These nucleic acids polymerize within the cartridge thereby forming strands which adhere to capture and reference sensors through electrostatic interactions between the strands and any pre-treatment reagents). The presence of these nucleic acid strands which adhere to the capture and reference sensors can cause errors in the results reported by an assay. The use of a solution (e.g., such as a pre-treatment solution) containing a nuclease in the methods described herein may: (a) prevent or disrupt the formation of such aggregates, particularly when the sample is a whole blood sample; and (b) when used in a cartridge in a point-of-care device, prevent the nucleic acid strands from adhering to one or more of the capture and/or reference sensor, thereby preventing errors from being reported by the assay. The use of a solution (e.g., such as a pre-treatment solution) containing a nuclease in the methods described herein may prevent or disrupt the formation of such aggregates, particularly when the sample is a whole blood sample, compared to a method that does not use a solution (e.g., such as a pre-treatment solution) containing a nuclease.

In some embodiments the nuclease is an endonuclease or nuclease. In some embodiments, the nuclease cleaves DNA (e.g., is a DNase). In some embodiments, the nuclease cleaves RNA (e.g., is an RNase), but preferably is a nuclease that cleaves RNA (i.e., an RNase) also must have DNA cleavage activity (i.e., must also be a DNase). In some embodiments, the nuclease cleaves DNA and RNA. In some embodiments, the nuclease is a Benzonase® nuclease. In yet other embodiments, the solution comprising a nuclease comprises at least about 1 U/mL nuclease, such as, for example, a Benzonase® nuclease. In some embodiments, the solution comprising a nuclease comprises about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease. In still other embodiments, the final concentration or amount of nuclease used in the methods described herein is about 1.0 U/mL of nuclease. In still further embodiments, the final concentration or amount of Benzonase® nuclease used in the methods described herein is about 1.0 U/mL. In yet further embodiments, when included in a cartridge for use in a point-of-care device, the amount of nuclease (e.g., Benzonase® nuclease) included in a pre-treatment reagent is about 1.5 U/mL. Once the pre-treatment reagent containing a nuclease is mixed with the biological sample, the final concentration of nuclease in the sample is about 1.0 U/mL.

In some embodiments, the solution comprising a nuclease does not contain heparin. It has also been found that avoiding the use of heparin in the solution comprising the nuclease can further aid in preventing or disrupting the formation of aggregates that may otherwise occur during performance of the assay. In some embodiments, the method does not use any heparin.

In some embodiments, the method comprises contacting the sample with the solution comprising a nuclease before contacting the sample with the capture antibody and/or the detection antibody. For example, the sample may be contacted with the solution comprising the nuclease as a pre-treatment step, or as part of a sample pre-treatment step. In yet other embodiments, the method comprises contacting the sample with the solution comprising a nuclease at the same time as contacting the sample with the capture antibody and/or detection antibody. In still yet other embodiments, the method comprises contacting the sample comprising a nuclease after contacting the sample with the capture antibody and/or detection antibody.

In some embodiments, the method comprises contacting the sample with the solution comprising the nuclease before contacting the sample with the capture antibody. In some embodiments, the method comprises contacting the sample with the solution comprising a nuclease after contacting the sample with the capture antibody (e.g., after formation of the capture antibody-biomarker complex). In some embodiments, the method comprises contacting the sample with the solution comprising a nuclease after contacting the sample with the capture antibody and the detection antibody (e.g., after formation of the capture antibody-biomarker-detection antibody complex). In still other embodiments, the method comprises contacting the sample with the solution comprising the nuclease at the same time the sample is contacted with the capture antibody and/or detection antibody. In still other embodiments, the method comprises contacting the sample with the solution comprising the nuclease after the sample is contacted with the capture antibody and/or detection antibody (e.g., after complex formation). In some embodiments, the method further comprises measuring the amount or concentration of the biomarker in the sample based upon the signal generated by the detectable label in the capture antibody-biomarker-detection antibody complex.

In some embodiments, the biomarker is GFAP, optionally used as an ABI or TBI biomarker. Accordingly, in some embodiments the assay comprises contacting the sample, either simultaneously or sequentially, in any order, with: (1) one or more capture antibodies which bind to an epitope on GFAP to form a capture antibody-GFAP antigen-complex, (2) one or more detection antibodies which bind to an epitope on GFAP that is not bound by the capture antibody to form a capture antibody-GFAP antigen-detection antibody complex, and (3) a solution comprising a nuclease. Either the capture antibody or the detection antibody may comprise a detectable label. In some embodiments, the method comprises contacting the sample with the solution comprising a nuclease (e.g., an endonuclease) after contacting the sample with one or more capture antibodies (e.g., after formation of the capture antibody-GFAP antigen-complex). In some embodiments, the method comprises contacting the sample with the solution comprising a nuclease (e.g., an endonuclease) after contacting the sample with the one or more capture antibodies and the one or more detection antibodies (e.g., after formation of the capture antibody-GFAP antigen-detection antibody complex). In some embodiments, the method further comprises measuring the amount or concentration of GFAP in the sample based upon the signal generated by the detectable label in the capture antibody-GFAP antigen-detection antibody complex. In some embodiments, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In some embodiments, the solution comprising a nuclease does not contain heparin. In yet other embodiments, the solution comprising a nuclease further comprises one or more salts, such as magnesium chloride.

In some embodiments, the biomarker is UCH-L1, optionally used as an ABI or TBI biomarker. Accordingly, in some embodiments the assay comprises contacting the sample, either simultaneously or sequentially, in any order, with: (1) one or more capture antibodies which bind to an epitope on UCH-L1 to form a capture antibody-UCH-L1 antigen-complex, (2) one or more detection antibodies which bind to an epitope on UCH-L1 that is not bound by the capture antibody to form a capture antibody-UCH-L1 antigen-detection antibody complex, and (3) a solution comprising a nuclease. Either the capture antibody or the detection antibody may comprise a detectable label. In some embodiments, the method comprises contacting the sample with a solution comprising a nuclease (e.g., an endonuclease) before contacting the sample with the capture antibody. In some embodiments, the method comprises contacting the sample with the solution comprising a nuclease (e.g., an endonuclease) after contacting the sample with the capture antibody (e.g., after formation of the capture antibody-UCH-L1 antigen-complex). In still other embodiments, the method comprises contacting the sample with a solution comprising a nuclease (e.g., an endonuclease) at the same time as contacting the sample with the capture antibody and/or detection antibody. In some embodiments, the method comprises contacting the sample with the solution comprising a nuclease (e.g., an endonuclease) after contacting the sample with the capture antibody and the detection antibody (e.g., after formation of the capture antibody-UCH-L1 antigen-detection antibody complex). In some embodiments, the method further comprises measuring the amount or concentration of UCH-L1 in the sample based upon the signal generated by the detectable label in the capture antibody-UCH-L1 antigen-detection antibody complex. In some embodiments, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In some embodiments, the solution comprising a nuclease does not contain heparin. In some embodiments, the method does not use any heparin.

### 5. Methods and Systems of Determining Whether a Subject's Levels of an ABI Biomarker are Elevated

In some embodiments, the disclosure relates to methods and systems of determining whether a subject's levels of an ABI biomarker, such as a TBI biomarker (e.g., GFAP, UCH-L1, or GFAP and UCH-L1) are elevated. In some embodiments, the methods and systems for determining whether a subject's levels of a TBI biomarker (e.g., GFAP, UCH-L1, or GFAP and UCH-L1) are elevated aid in the diagnosis and evaluation of whether the subject has sustained an injury to the head. In some embodiments, the methods and systems for determining whether a subject's levels of a TBI biomarker (e.g., GFAP, UCH-L1, or GFAP and UCH-L1) are elevated can aid in the determination of whether or not a subject requires further evaluation, such as by a head computed tomography (CT) scan and/or a magnetic resonance imaging (MRI) procedure.

In some embodiments, methods for determining whether a subject's level of at least one ABI or TBI biomarker is elevated involves performing an assay described herein, including the assays described above in section 3. In some embodiments, the assay comprises contacting the sample, either simultaneously or sequentially, in any order, with: (1) a capture antibody which binds to an epitope on the TBI biomarker to form a capture antibody-biomarker-complex, (2) a detection antibody which binds to an epitope on the TBI biomarker that is not bound by the capture antibody to form a capture antibody-biomarker-detection antibody complex, and (3) a solution comprising a nuclease salts. In some embodiments, the method comprises performing at least one assay for UCH-L1, at least one assay for GFAP, or at least one assay for UCH-L1 and at least one assay for GFAP in at least one sample obtained from the subject (e.g., from the human subject). In some embodiments, the sample is obtained within about 48 hours after an actual or suspected injury to the head. In other embodiments, the sample is obtained within about 24 hours after an actual or suspected injury to the head. In yet other embodiments, the sample is obtained within about 12 hours after an actual or suspected injury to the head. The method comprises determining whether the subject's levels of GFAP, UCH-L1, or GFAP and UCH-L1 are elevated based upon a comparison of the level of GFAP in the sample to a reference level of GFAP, the level of UCH-L1 in the sample to a reference level of UCH-L1, or the level of GFAP in the sample to a reference level of GFAP and the level of UCH-L1 in the sample to a reference level of UCH-L1.

In some embodiments, the method can include obtaining a sample within about 48 hours (e.g., within about 48 hours, within about 24 hours, or within about 12 hours) of an actual or suspected injury to the subject and contacting the sample with an antibody for the biomarker ubiquitin carboxy-terminal hydrolase L1 (UCH-L1) and/or an antibody for the biomarker glial fibrillary acidic protein (GFAP), to allow formation of a complex of the antibody and the biomarker. The method also includes detecting the resulting antibody-biomarker complex or complexes.

In some embodiments, the sample is taken from the subject (e.g., human subject) within about 48 hours of injury of an actual or suspected injury to the head. For example, the sample can be taken from the subject (e.g., a human subject) within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In still other embodiments, the sample is taken within about 8 hours to within about 48 hours after the actual or suspected injury to the head. In still other embodiments, the sample is taken within about 9 hours to within about 48 hours after the actual or suspected injury to the head. In still other embodiments, the sample is taken within about 10 hours to within about 48 hours after the actual or suspected injury to the head. In still other embodiments, the sample is taken within about 11 hours to within about 48 hours after the actual or suspected injury to the head. In still other embodiments, the sample is taken within about 12 hours to within about 48 hours after the actual or suspected injury to the head. In still other embodiments, the sample is taken within about 13 hours to within about 48 hours after the actual or suspected injury to the head. In still other embodiments, the sample is taken within about 14 hours to within about 48 hours after the actual or suspected injury to the head. In still other embodiments, the sample is taken within about 15 hours to within about 48 hours after the actual or suspected injury to the head. In still other embodiments, the sample is taken within about 16 hours to within about 48 hours after the actual or suspected injury to the head. In still other embodiments, the sample is taken within about 17 hours to within about 48 hours after the actual or suspected injury to the head. In still other embodiments, the sample is taken within about 18 hours to within about 48 hours after the actual or suspected injury to the head. In still other embodiments, the sample is taken within about 19 hours to within about 48 hours after the actual or suspected injury to the head. In still other embodiments, the sample is taken within about 20 hours to within about 48 hours after the actual or suspected injury to the head. In still other embodiments, the sample is taken within about 21 hours to within about 48 hours after the actual or suspected injury to the head. In still other embodiments, the sample is taken within about 22 hours to within about 48 hours after the actual or suspected injury to the head. In still other embodiments, the sample is taken within about 23 hours to within about 48 hours after the actual or suspected injury to the head. In still other embodiments, the sample is taken within about 24 hours to within about 48 hours after the actual or suspected injury to the head. In still other embodiments, the sample is taken within about 25 hours to within about 48 hours after the actual or suspected injury to the head. In still other embodiments, the sample is taken within about 26 hours to within about 48 hours after the actual or suspected injury to the head. In still other embodiments, the sample is taken within about 27 hours to within about 48 hours after the actual or suspected injury to the head. In still other embodiments, the sample is taken within about 28 hours to within about 48 hours after the actual or suspected injury to the head. In still other embodiments, the sample is taken within about 29 hours to within about 48 hours after the actual or suspected injury to the head. In still other embodiments, the sample is taken within about 30 hours to within about 48 hours after the actual or suspected injury to the head. In still other embodiments, the sample is taken within about 31 hours to within about 48 hours after the actual or suspected injury to the head. In still other embodiments, the sample is taken within about 32 hours to within about 48 hours after the actual or suspected injury to the head. In still other embodiments, the sample is taken within about 33 hours to within about 48 hours after the actual or suspected injury to the head. In still other embodiments, the sample is taken within about 34 hours to within about 48 hours after the actual or suspected injury to the head. In still other embodiments, the sample is taken within about 35 hours to within about 48 hours after the actual or suspected injury to the head. In still other embodiments, the sample is taken within about 36 hours to within about 48 hours after the actual or suspected injury to the head. In still other embodiments, the sample is taken within about 37 hours to within about 48 hours after the actual or suspected injury to the head. In still other embodiments, the sample is taken within about 38 hours to within about 48 hours after the actual or suspected injury to the head. In still other embodiments, the sample is taken within about 39 hours to within about 48 hours after the actual or suspected injury to the head. In still other embodiments, the sample is taken within about 40 hours to within about 48 hours after the actual or suspected injury to the head.

In some embodiments, the onset of the presence of the biomarker, such as UCH-L1, GFAP, or a combination thereof, appears within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In other embodiments, the onset of the presence of the biomarker, such as UCH-L1, GFAP, or a combination thereof, appears within about 8 hours to within about 48 hours, within about 9 hours to within about 48 hours, within about 10 hours to within about 48 hours, within about 11 hours to within about 48 hours, within about 12 hours to within about 48 hours, within about 13 hours to within about 48 hours, within about 14 hours to within about 48 hours, within about 15 hours to within about 48 hours, within about 16 hours to within about 48 hours, within about 17 hours to within about 48 hours, within about 18 hours to within about 48 hours, within about 19 hours to within about 48 hours, within about 20 hours to within about 48 hours, within about 21 hours to within about 48 hours, within about 22 hours to within about 48 hours, within about 23 hours to within about 48 hours, within about 24 hours to within about 48 hours, 25 hours to within about 48 hours, within about 26 hours to within about 48 hours, within about 27 hours to within about 48 hours, within about 29 hours to within about 48 hours, within about 30 hours to within about 48 hours, within about 31 hours to within about 48 hours, within about 32 hours to within about 48 hours, within about 33 hours to within about 48 hours, within about 34 hours to within about 48 hours, within about 35 hours to within about 48 hours, within about 36 hours to within about 48 hours, within about 37 hours to within about 48 hours, within about 38 hours to within about 48 hours, within about 39 hours to within about 48 hours, or within about 40 hours to within about 48 hours, after an actual or suspected injury to the head.

In some embodiments, the method comprises performing at least one assay for a TBI biomarker, and determining whether the subject's levels of the TBI biomarker are elevated based upon the results of the assay. In some embodiments, the levels are determined to be elevated. In some embodiments, the levels are determined to not be elevated.

In some embodiments, the method comprises performing at least one assay for UCH-L1, at least one assay for GFAP, or at least one assay for UCH-L1 and at least one assay for GFAP in at least one sample obtained from the subject, and determining whether the subject's levels of UCH-L1, GFAP, or GFAP and UCH-L1 are elevated based upon the results of the assays. In some embodiments, the method comprises determining that the subject's levels of GFAP, UCH-L1, or GFAP and UCH-L1 are elevated. In some embodiments, the method comprises determining that the subject's levels of GFAP, UCH-L1, or GFAP and UCH-L1 are elevated when the level of GFAP alone in the sample is equal to or above about 30 μg/mL or 65 μg/mL, the level of UCH-L1 alone in the sample is equal to or about 360 μg/mL, the level of GFAP in the sample is equal to or about 30 μg/mL or 65 μg/mL and the the level of UCH-L1 is below about 360 μg/mL, or the level of GFAP in the sample is equal to or above about 30 μg/mL or 65 μg/mL and the level of UCH-L1 is below about 360 μg/mL, cannot be determined by the assay for UCH-L1, or is not reported by the assay for UCH-L1. In some embodiments, the method comprises determining that the subject's levels of GFAP, UCH-L1, or GFAP and UCH-L1 are elevated when the level of GFAP alone is equal to or above about 30 μg/mL or 65 μg/mL, the level of UCH-L1 alone is equal to or above about 360 μg/mL, or the level of GFAP is equal to or above about 30 μg/mL or 65 μg/mL and level of UCH-L1 is equal to or above about 360 μg/mL. In some embodiments, the method comprises determining that the subject's levels of GFAP and UCH-L1 are elevated when the level of GFAP cannot be determined by the assay for GFAP or is not reported by the assay for GFAP, and the level of UCH-L1 is equal to or above about 360 μg/mL.

In some embodiments, the method comprises determining that the subject's levels of GFAP, UCH-L1, or GFAP and UCH-L1 are not elevated. In some embodiments, the method comprises determining that the subject's levels of GFAP, UCH-L1, or GFAP and UCH-L1 are not elevated when the level of GFAP alone in the sample is below about 30 μg/mL or 65 μg/mL, the level of UCH-L1 alone in the sample is below about 360 μg/mL, or the level of GFAP in the sample is below about 30 μg/mL or 65 μg/mL and level of UCH-L1 in the sample is below about 360 μg/mL.

In some embodiments, the method comprises determining that the assays for UCH-L1, GFAP, or UCH-L1 and GFAP should be repeated. In some embodiments, the method comprises determining that the assays for UCH-L1, GFAP, or UCH-L1 and GFAP should be repeated when the level of UCH-L1 alone in the sample cannot be determined or is not reported, the level of GFAP is below about 30 μg/mL or 65 μg/mL and the level of UCH-L1 cannot be determined by the assay for UCH-L1 or is not reported by the assay for UCH-L1, or the level of GFAP alone in the sample cannot be determined or is not reported. In some embodiments, the method comprises determining that the assays for UCH-L1 and GFAP should be repeated when the level of GFAP cannot be determined by the assay for GFAP or is not reported by the assay for GFAP and the level of UCH-L1 is below about 360 μg/mL. In some embodiments, the method comprises determining that the assays for UCH-L1 and GFAP should be repeated when the level of GFAP cannot be determined by the assay for GFAP or is not reported by the assay for GFAP and the level of UCH-L1 cannot be determined by the UCH-L1 or is not reported by the assay for UCH-L1.

In some embodiments, the method comprises communicating the determination of whether the levels of the TBI biomarker are elevated, not elevated, or that assays need to be repeated from at least one instrument (e.g. a point-of-care device). In some embodiments, the method comprises communicating the determination (e.g. the determination that subject's levels of GFAP, UCH-L1, or GFAP and UCH-L1 are elevated, the determination that the subject's levels of GFAP, UCH-L1, or GFAP and UCH-L1 are not elevated, or the determination that the assays for GFAP, UCH-L1, or GFAP and UCH-L1 should be repeated) on or from at least one instrument. Suitable instruments are described herein, including point-of-care devices and non-point-of care devices that may contain a user interface that communicate by displaying the determination.

As discussed in further detail in Section 8, in some embodiments, the instrument contains software to execute one or more tasks. In some embodiments, the instrument contains software to automatically determine the next appropriate step in a method as described herein. For example, the instrument may contain software that determines whether levels of GFAP, UCH-L1, or GFAP and UCH-L1 are elevated, whether levels are not elevated, and/or whether the assays need to be repeated. The software may display this determination, such as on a graphical user interface.

In some embodiments, the instrument stores software that instructs a processor to execute a given task. In some embodiments, the software stores machine readable instructions that instruct a processor to execute a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine-readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine readable instructions and/or the corresponding program(s) can be executed in whole or in part. Thus, the disclosed machine-readable instructions and/or corresponding program(s) are intended to encompass such machine readable instructions and/or program(s) regardless of the particular format or state of the machine readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

In some embodiments, the method further comprises performing a head computed tomography (CT) scan, a magnetic resonance imaging (MRI) procedure, or both a CT scan or a MRI procedure on the subject when the subject's levels of the TBI biomarker are determined to be elevated. In some embodiments, the method comprises performing a head CT scan, an MRI procedure, or both a head CT scan and an MRI procedure when the subject's levels of GFAP, UCH-L1, or GFAP and UCH-L1 are elevated. For example, in some embodiments the method further comprises performing a head CT scan on the subject when the subject's levels of GFAP, UCH-L1, or GFAP and UCH-L1 are elevated. As another example, in some embodiments the method further comprises performing an MRI procedure on the subject when the subject's levels of GFAP, UCH-L1, or GFAP and UCH-L1 are elevated. In some embodiments, the method further comprises performing a head CT scan and an MRI procedure on the subject when the subject's levels of GFAP, UCH-L1, or GFAP and UCH-L1 are elevated.

In some embodiments, the method further comprises not performing a head computed tomography (CT) scan, a magnetic resonance imaging (MRI) procedure, or both a head CT scan or a MRI procedure on the subject when the subject's levels of the TBI biomarker (e.g., GFAP, UCH-L1, or GFAP and UCH-L1) are not elevated. In other words, the method involves “ruling out” the need for a head CT scan, an MRI procedure or both when the subject's TBI biomarker levels (e.g. GFAP, UCH-L1, or GFAP and UCH-L1 levels) are not elevated.

In some embodiments, the method further comprises diagnosing the subject as having a traumatic brain injury (TBI) based upon the levels of the TBI biomarker. In some embodiments, the method comprises diagnosing the subject as having a TBI when the level of GFAP alone is equal to or above about 30 μg/mL or 65 μg/mL, the level of UCH-L1 alone is equal to or above about 360 μg/mL, or the level of GFAP is equal to or above about 30 μg/mL or 65 μg/mL and the level of UCH-L1 is equal to or above about 360 μg/mL, regardless of whether a head CT scan is negative for a TBI or whether any head CT scan is performed.

In some embodiments, the method further comprises treating the subject for a mild, moderate, moderate to severe, or severe TBI when the subject's levels of the TBI biomarker are determined to be elevated. In some embodiments, the method further comprises treating the subject for a mild, moderate to severe, or severe TBI when the subject's levels of GFAP, UCH-L1, or GFAP and UCH-L1 are determined to be elevated. For example, in some embodiments the method further comprises treating the subject for a mild TBI when the subject's levels of GFAP, UCH-L1, or GFAP and UCH-L1 are determined to be elevated. In some embodiments, the method further comprises treating the subject for a moderate to severe TBI when the subject's levels of GFAP, UCH-L1, or GFAP and UCH-L1 are determined to be elevated. In some embodiments, the method further comprises treating the subject for a severe TBI when the subject's levels of GFAP, UCH-L1, or GFAP and UCH-L1 are determined to be elevated. In some embodiments, selection of the appropriate treatment may be facilitated by results from a head CT scan, an MRI procedure, or both, if performed on the subject. For example, results from a head CT scan and/or MRI procedure may help in further differentiating between a mild, moderate to severe, or a severe TBI in the subject. Such a differentiation may assist in selection of the appropriate treatment for the subject. In some embodiments, the method further comprises monitoring the subject when the subject's levels of GFAP, UCH-L1, or GFAP and UCH-L1 are elevated.

In some embodiments, the method further includes treating a subject (e.g., a human subject) assessed as having mild, moderate, severe, or moderate to severe traumatic brain injury with a traumatic brain injury treatment, as described below. In yet other embodiments, the method further includes treating a subject (e.g., a human subject) assessed with a mild traumatic brain injury with traumatic brain injury treatment, as described below. In yet other embodiments, the method further includes treating a subject (e.g., a human subject) assessed with moderate traumatic brain injury with traumatic brain injury treatment, as described below. In yet other embodiments, the method further includes treating a subject assessed with severe traumatic brain injury with a traumatic brain injury treatment. In some embodiments, the method further includes monitoring a subject (e.g., a human subject) assessed as having mild traumatic brain injury, as described below. In other embodiments, the method further includes monitoring a subject (e.g., a human subject) assessed as having a moderate traumatic brain injury, as described below. In yet other embodiments, the method further includes monitoring a subject (e.g., a human subject) assessed as having a severe traumatic brain injury, as described below. In yet other embodiments, the method further includes monitoring a subject (e.g., a human subject) assessed as having a moderate to severe traumatic brain injury.

In some embodiments, the method comprises performing an assay for a first TBI biomarker, and an assay for a second TBI biomarker. The assays may be performed simultaneously or sequentially. For example, in some embodiments the method comprises performing an assay for GFAP and an assay for UCH-L1. The at least one assay for GFAP and the at least one assay for UCH-L1 may be performed simultaneously. Alternatively, the assay for GFAP and the assay for UCH-L1 may be performed sequentially. The assays may be performed sequentially, in any order. For example, the assay for GFAP may be performed first, followed by the assay for UCH-L1. As another example, the assay for UCH-L1 may be performed first, followed by the assay for GFAP.

In some embodiments, the assay (e.g., the assay for the TBI biomarker) is performed in about 10 to about 20 minutes. In some embodiments, the assay is performed in about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, the at least one assay for GFAP and/or at the at least one assay for UCH-L1 are each performed in about 10 to about 20 minutes. In some embodiments, the at least one assay for GFAP and/or at the at least one assay for UCH-L1 are each performed in about 10 minutes. In some embodiments, the at least one assay for GFAP and/or at the at least one assay for UCH-L1 are each performed in about 11 minutes. In some embodiments, the at least one assay for GFAP and/or at the at least one assay for UCH-L1 are each performed in about 12 minutes. In some embodiments, the at least one assay for GFAP and/or at the at least one assay for UCH-L1 are each performed in about 13 minutes. In some embodiments, the at least one assay for GFAP and/or at the at least one assay for UCH-L1 are each performed in about 14 minutes. In some embodiments, the at least one assay for GFAP and/or at the at least one assay for UCH-L1 are each performed in about 15 minutes. In some embodiments, the at least one assay for GFAP and/or at the at least one assay for UCH-L1 are each performed in about 16 minutes. In some embodiments, the at least one assay for GFAP and/or at the at least one assay for UCH-L1 are each performed in about 17 minutes. In some embodiments, the at least one assay for GFAP and/or at the at least one assay for UCH-L1 are each performed in about 18 minutes. In some embodiments, the at least one assay for GFAP and/or at the at least one assay for UCH-L1 are each performed in about 19 minutes. In some embodiments, the at least one assay for GFAP and/or at the at least one assay for UCH-L1 are each performed in about 20 minutes.

Tests or assays competent to perform the claimed methods will be employed, such as, for example, assays having various sensitivities and sensitivities as described herein. Moreover, the assays employed in the methods described herein can be employed in a clinical chemistry format such as would be known by one of ordinary skill in the art. Such assays are described in further detail herein. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4th edition, chapter 3.5, pages 315-322, the contents of which are herein incorporated by reference).

### 6. Methods of Aiding in the Diagnosis and Evaluation of Whether a Subject has Sustained or is Suspected of Having Sustained an Injury to the Head Using a Reference Level

In yet another embodiment, the present disclosure relates, among other methods, to an improved method of evaluating or aiding in the diagnosis and evaluation of whether a subject (e.g., human subject) has sustained or may have sustained an injury to the head. In some embodiments, the methods for determining whether a subject's levels of an ABI biomarker, such as, a TBI biomarker (e.g., GFAP, UCH-L1, or GFAP and UCH-L1) are elevated can assist in the determination of whether a subject has sustained an acquired brain injury, such as a traumatic brain injury. In some embodiments, methods for determining whether a subject's level of an ABI biomarker is elevated involve performing one or more of the assays described above in section 3.

In some embodiments, these method can aid in determining the extent of traumatic brain injury in a subject (e.g., human subject) with an actual or suspected injury to the head, e.g., determining whether the subject (e.g., a human subject) has a mild traumatic brain injury, moderate traumatic brain injury, severe traumatic brain injury, or a moderate to severe traumatic brain injury. As used herein, “determining whether the subject (e.g., a human subject) has a mild traumatic brain injury, a moderate traumatic brain injury, a severe traumatic brain injury, or a moderate to severe brain injury” refers to the fact that the aforementioned method can be used, e.g., with other information (e.g., clinical assessment data), to determine that the subject is more likely than not to have a mild traumatic brain injury, moderate traumatic brain injury, severe traumatic brain injury, or moderate to severe traumatic brain injury. The method can include performing an assay on a sample obtained from the subject (e.g., a human subject) within about 48 hours after an actual or suspected injury to the head to measure or detect a levels of a TBI biomarker (e.g., ubiquitin carboxy-terminal hydrolase L1 (UCH-L1) and/or glial fibrillary acidic protein (GFAP)) in the sample and determining whether the subject (e.g., a human subject) has sustained a mild, moderate, severe, or a moderate to severe traumatic brain injury (TBI) based upon the levels of the TBI biomarker (e.g., GFAP, UCH-L1, or GFAP and UCH-L1). In some embodiments, the method can include performing an assay, such as those described in Section 3, on a sample obtained from the subject (e.g., a human subject) within about 24 hours after an actual or suspected injury to the head to measure or detect a levels of ubiquitin carboxy-terminal hydrolase L1 (UCH-L1) and/or glial fibrillary acidic protein (GFAP) in the sample and determining whether the subject (e.g., a human subject) has sustained a mild, moderate, severe, or a moderate to severe traumatic brain injury (TBI) based upon the levels of GFAP, UCH-L1, or GFAP and UCH-L1. In other embodiments, the method can include performing an assay, such as those described in Section 3, on a sample obtained from the subject (e.g., a human subject) within about 12 hours after an actual or suspected injury to the head to measure or detect a levels of ubiquitin carboxy-terminal hydrolase L1 (UCH-L1) and/or glial fibrillary acidic protein (GFAP) in the sample and determining whether the subject (e.g., a human subject) has sustained a mild, moderate, severe, or a moderate to severe traumatic brain injury (TBI) based upon the levels of GFAP, UCH-L1, or GFAP and UCH-L1. In some embodiments, the subject is determined as having a mild, moderate, severe, or moderate or severe TBI based upon the determination of whether the levels of GFAP, UCH-L1, or GFAP and UCH-L1 are elevated in the sample obtained from the subject. In some embodiments, the subject is determined as having a mild, moderate, severe, or moderate to severe TBI when the levels of GFAP, UCH-L1, or GFAP and UCH-L1 are determined to be elevated. In some embodiments, determination of whether levels of GFAP, UCH-L1, or GFAP and UCH-L1 is dependent on comparing the level of GFAP in the sample to a reference level for GFAP, the level of UCH-L1 in the sample to a reference level for UCH-L1, or the level of GFAP in the sample to a reference level for GFAP and comparing the level of UCH-L1 in the sample to a reference level for UCH-L1. The sample can be a biological sample.

In some embodiments, the method can include obtaining a sample within about 48 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. In other embodiments, the method can include obtaining a sample within about 24 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. In yet further embodiments, the method can include obtaining a sample within about 12 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. The method also includes detecting the resulting antibody-biomarker complex.

In some embodiments, the sample is taken from the subject (e.g., human subject) within about 48 hours of injury of an actual or suspected injury to the head. For example, the sample can be taken from the subject (e.g., a human subject) within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In other embodiments, the sample is taken within about 8 hours to within about 48 hours after the actual or suspected injury to the head. In still other embodiments, the sample is taken within about 9 hours to within about 48 hours after the actual or suspected injury to the head. In still other embodiments, the sample is taken within about 10 hours to within about 48 hours after the actual or suspected injury to the head. In still other embodiments, the sample is taken within about 11 hours to within about 48 hours after the actual or suspected injury to the head. In still other embodiments, the sample is taken within about 12 hours to within about 48 hours after the actual or suspected injury to the head. In still other embodiments, the sample is taken within about 13 hours to within about 48 hours after the actual or suspected injury to the head. In still other embodiments, the sample is taken within about 14 hours to within about 48 hours after the actual or suspected injury to the head. In still other embodiments, the sample is taken within about 15 hours to within about 48 hours after the actual or suspected injury to the head. In still other embodiments, the sample is taken within about 16 hours to within about 48 hours after the actual or suspected injury to the head. In still other embodiments, the sample is taken within about 17 hours to within about 48 hours after the actual or suspected injury to the head. In still other embodiments, the sample is taken within about 18 hours to within about 48 hours after the actual or suspected injury to the head. In still other embodiments, the sample is taken within about 19 hours to within about 48 hours after the actual or suspected injury to the head. In still other embodiments, the sample is taken within about 20 hours to within about 48 hours after the actual or suspected injury to the head. In still other embodiments, the sample is taken within about 21 hours to within about 48 hours after the actual or suspected injury to the head. In still other embodiments, the sample is taken within about 22 hours to within about 48 hours after the actual or suspected injury to the head. In still other embodiments, the sample is taken within about 23 hours to within about 48 hours after the actual or suspected injury to the head. In still other embodiments, the sample is taken within about 24 hours to within about 48 hours after the actual or suspected injury to the head. In still other embodiments, the sample is taken within about 25 hours to within about 48 hours after the actual or suspected injury to the head. In still other embodiments, the sample is taken within about 26 hours to within about 48 hours after the actual or suspected injury to the head. In still other embodiments, the sample is taken within about 27 hours to within about 48 hours after the actual or suspected injury to the head. In still other embodiments, the sample is taken within about 28 hours to within about 48 hours after the actual or suspected injury to the head. In still other embodiments, the sample is taken within about 29 hours to within about 48 hours after the actual or suspected injury to the head. In still other embodiments, the sample is taken within about 30 hours to within about 48 hours after the actual or suspected injury to the head. In still other embodiments, the sample is taken within about 31 hours to within about 48 hours after the actual or suspected injury to the head. In still other embodiments, the sample is taken within about 32 hours to within about 48 hours after the actual or suspected injury to the head. In still other embodiments, the sample is taken within about 33 hours to within about 48 hours after the actual or suspected injury to the head. In still other embodiments, the sample is taken within about 34 hours to within about 48 hours after the actual or suspected injury to the head. In still other embodiments, the sample is taken within about 35 hours to within about 48 hours after the actual or suspected injury to the head. In still other embodiments, the sample is taken within about 36 hours to within about 48 hours after the actual or suspected injury to the head. In still other embodiments, the sample is taken within about 37 hours to within about 48 hours after the actual or suspected injury to the head. In still other embodiments, the sample is taken within about 38 hours to within about 48 hours after the actual or suspected injury to the head. In still other embodiments, the sample is taken within about 39 hours to within about 48 hours after the actual or suspected injury to the head. In still other embodiments, the sample is taken within about 40 hours to within about 48 hours after the actual or suspected injury to the head.

In some embodiments, the onset of the presence of the biomarker, such as UCH-L1, GFAP, or a combination thereof, appears within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In other embodiments, the onset of the presence of the biomarker, such as UCH-L1, GFAP, or a combination thereof, appears within about 8 hours to within about 48 hours, within about 9 hours to within about 48 hours, within about 10 hours to within about 48 hours, within about 11 hours to within about 48 hours, within about 12 hours to within about 48 hours, within about 13 hours to within about 48 hours, within about 14 hours to within about 48 hours, within about 15 hours to within about 48 hours, within about 16 hours to within about 48 hours, within about 17 hours to within about 48 hours, within about 18 hours to within about 48 hours, within about 19 hours to within about 48 hours, within about 20 hours to within about 48 hours, within about 21 hours to within about 48 hours, within about 22 hours to within about 48 hours, within about 23 hours to within about 48 hours, within about 24 hours to within about 48 hours, 25 hours to within about 48 hours, within about 26 hours to within about 48 hours, within about 27 hours to within about 48 hours, within about 29 hours to within about 48 hours, within about 30 hours to within about 48 hours, within about 31 hours to within about 48 hours, within about 32 hours to within about 48 hours, within about 33 hours to within about 48 hours, within about 34 hours to within about 48 hours, within about 35 hours to within about 48 hours, within about 36 hours to within about 48 hours, within about 37 hours to within about 48 hours, within about 38 hours to within about 48 hours, within about 39 hours to within about 48 hours, or within about 40 hours to within about 48 hours, after an actual or suspected injury to the head.

In some embodiments, the subject has received a Glasgow Coma Scale score before or after the assay is performed. In some embodiments, the subject (e.g., a human subject) is suspected as having moderate, severe, or moderate to severe traumatic brain injury based on the Glasgow Coma Scale score. In some embodiments, the reference level of the biomarker, such as UCH-L1, GFAP, or a combination thereof, is correlated with subjects having moderate, severe, or moderate to severe traumatic brain injury. In some embodiments, the reference level of the biomarker, such as UCH-L1, GFAP, or a combination thereof, is correlated with a Glasgow Coma Scale score of 9-13 (a moderate TBI). In some embodiments, the reference level of the biomarker, such as UCH-L1, GFAP, or a combination thereof, is correlated with a Glasgow Coma Scale score of 3-8 (a severe TBI). In some embodiments, the reference level of the biomarker, such as UCH-L1, GFAP, or a combination thereof, is correlated with a Glasgow Coma Scale score of 3-12 (a moderate, severe, or moderate to severe TBI). In some embodiments, the subject is suspected as having mild traumatic brain injury based on the Glasgow Coma Scale score. In some embodiments, the reference level of the biomarker, such as UCH-L1, GFAP, or a combination thereof, is correlated with subjects having mild traumatic brain injury. In some embodiments, the reference level of the biomarker, such as UCH-L1, GFAP, or a combination thereof, is correlated with a Glasgow Coma Scale score of 13-15 (mild TBI).

Generally, a reference level of the biomarker, such as UCH-L1, GFAP, or a combination thereof, can also be employed as a benchmark against which to assess results obtained upon assaying a test sample for the biomarker, such as UCH-L1, GFAP, or a combination thereof. Generally, in making such a comparison, the reference level of the biomarker, such as UCH-L1, GFAP, or a combination thereof, is obtained by running or conducting a particular assay a sufficient number of times and under appropriate conditions such that a linkage or association of analyte presence, amount or concentration with a particular stage or endpoint of TBI or with particular indicia can be made. Typically, the reference level of the biomarker, such as UCH-L1, GFAP, or a combination thereof, is obtained with assays of reference subjects (or populations of subjects). The biomarker, such as UCH-L1, GFAP, or a combination thereof, measured can include fragments thereof, degradation products thereof, and/or enzymatic cleavage products thereof.

In certain embodiments, the reference level may be correlated with control subjects (e.g., human subjects) that have not sustained a head injury.

In still yet further embodiments, the method comprises determining that the subject has a traumatic brain injury when the subject's levels of the TBI biomarker are elevated. For example, in some embodiments the method comprises determining that the subject has a TBI when the GFAP, UCH-L1, or GFAP and UCH-L1 are elevated. For example, in some embodiments the method comprises determining that the subject has a mild, moderate, severe, or moderate to severe traumatic brain injury when the level of GFAP alone in the sample obtained from the subject is equal to or above the threshold value of about 30 μg/mL or 65 μg/mL, the level of GFAP in the sample obtained from the subject is equal to or above the threshold value of about 30 μg/mL or 65 μg/mL and the level of UCH-L1 is below the threshold value of about 360 μg/mL, cannot be determined, or is not reported. In some embodiments, the method comprises determining that the subject has a mild, moderate, severe, or moderate to severe traumatic brain injury when the level of UCH-L1 alone in the sample is equal to or above the threshold value of about 360 μg/mL or the level of GFAP in the sample obtained from the subject is equal to or above the threshold value of about 30 μg/mL or 65 μg/mL and the level of UCH-L1 in the sample is equal to or above the threshold value of about 360 μg/mL. In some embodiments, the method comprises determining that the subject has a mild, moderate, severe, or moderate to severe traumatic brain injury when the level of GFAP in the sample obtained from the subject cannot be determined or is not reported and the level of UCH-L1 in the sample is equal to or above the threshold value of about 360 μg/mL.

In some embodiments, the method comprises determining that the subject likely does not have a traumatic brain injury when the subject's levels of the TBI biomarker (e.g., GFAP, UCH-L1, or GFAP and UCH-L1) are not elevated. For example, in some embodiments the method comprises determining that the subject likely does not have a traumatic brain injury when the level of GFAP alone in the sample is below the threshold level of about 30 μg/mL or 65 μg/mL, the level of UCH-L1 alone in the sample is below the threshold level of about 360 μg/mL, or the level of GFAP in the sample obtained from the subject is below the threshold value of about 30 μg/mL or 65 μg/mL and when the level of UCH-L1 in the sample is below the threshold value of about 360 μg/mL.

In some embodiments, the method further includes treating a subject (e.g. a human subject) assessed as having mild, moderate, severe, or moderate to severe traumatic brain injury with a traumatic brain injury treatment, as described below. In yet other embodiments, the method further includes treating a subject (e.g., a human subject) assessed with a mild traumatic brain injury with traumatic brain injury treatment, as described below. In yet other embodiments, the method further includes treating a subject (e.g., a human subject) assessed with moderate traumatic brain injury with traumatic brain injury treatment, as described below. In yet other embodiments, the method further includes treating a subject assessed with severe traumatic brain injury with a traumatic brain injury treatment. In some embodiments, the method further includes monitoring a subject (e.g., a human subject) assessed as having mild traumatic brain injury, as described below. In other embodiments, the method further includes monitoring a subject (e.g., a human subject) assessed as having a moderate traumatic brain injury, as described below. In yet other embodiments, the method further includes monitoring a subject (e.g., a human subject) assessed as having a severe traumatic brain injury, as described below. In yet other embodiments, the method further includes monitoring a subject (e.g., a human subject) assessed as having a moderate to severe traumatic brain injury.

Tests or assays competent to perform the claimed methods will be employed, such as, for example, assays having various sensitivities and sensitivities as described herein. Moreover, the assays employed in the methods described herein can be employed in a clinical chemistry format such as would be known by one of ordinary skill in the art. Such assays are described in further detail herein. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4th edition, chapter 3.5, pages 315-322, the contents of which are herein incorporated by reference).

### 7. Methods of Aiding in the Determination of Whether to Perform a CT Scan and/or MRI on a Subject Who has Sustained or May have Sustained an Injury to the Head Using a Reference Level

The present disclosure relates, among other methods, to an improved method of aiding in determining whether to perform a computerized tomography (CT) scan and/or magnetic resonance imaging on a subject (e.g., a human subject) who has sustained or may have sustained an actual or suspected injury to the head. In some embodiments, the methods for determining whether a subject's levels of an ABI biomarker, such as a TBI biomarker (e.g., GFAP, UCH-L1, or GFAP and UCH-L1) are elevated can assist in the determination of whether to perform a CT scan or MRI on a subject. In some embodiments, methods for determining whether to perform a CT scan and/or magnetic resonance imaging on a subject who has sustained or may have sustained an actual or suspected injury to the head involve performing one or more of the assays described above in section 3.

As used herein, “determination of whether to perform a CT scan on a subject” refers to the fact that the aforementioned method can be used, e.g., with other information (e.g., clinical assessment data), to determine that the subject (e.g., a human subject) is more likely than not to have a positive head CT scan. As used herein, “determination of whether to perform a MRI on a subject” refers to the fact that the aforementioned method can be used, e.g., with other information (e.g., clinical assessment data), to determine that the subject (e.g., a human subject) is more likely than not to have a positive head MRI scan. Specifically, such a method can comprise the steps of: (a) performing an assay on a sample obtained from the subject within about 48 hours after an actual or suspected injury to the head to determine whether the subject's levels of a biomarker of TBI (e.g. GFAP, UCH-L1, or GFAP and UCH-L1) are elevated using an assay described herein; and (b) determining whether to perform a CT scan and/or a MRI on the subject (e.g., a human subject) based upon whether the subject's levels of the TBI biomarker are elevated. In some embodiments, the assay, such as those described in Section 3, is performed on a sample obtained from the subject within about 24 hours after an actual or suspected injury to the head. In yet further embodiments, the assay, such as those described in Example 3, is performed on a sample obtained from the subject within about 12 hours after the actual or suspected injury to the head. In some embodiments, the method comprises performing a head CT scan or an MRI procedure on the subject when the levels of the TBI biomarker (e.g., GFAP, UCH-L1, or GFAP and UCH-L1) are determined to be elevated. In some embodiments, a CT scan is performed on the subject. In other embodiments, a MRI procedure is performed on the subject. In yet further embodiments, a CT scan and MRI is performed on the subject (the order in which the CT scan and MRI is performed is not critical). In some embodiments, the method comprises not performing a head CT scan or an MRI procedure on the subject when the levels of the biomarker of TBI are not determined to be elevated. In other words, the method involves “ruling out” the need for a head CT scan, an MRI procedure or both when the subject's GFAP, UCH-L1, or GFAP and UCH-L1 levels are not elevated. The sample can be a biological sample.

In some embodiments, the method can include obtaining a sample (e.g., a human subject) within about 48 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. In some embodiments, the method can include obtaining a sample (e.g., a human subject) within about 24 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. In some embodiments, the method can include obtaining a sample (e.g., a human subject) within about 12 hours of an actual or suspected injury to the subject and contacting the sample with an antibody for a TBI biomarker, such as ubiquitin carboxy-terminal hydrolase L1 (UCH-L1), glial fibrillary acidic protein (GFAP), or a combination thereof, to allow formation of a complex of the antibody and the biomarker. The method also includes detecting the resulting antibody-biomarker complex.

In some embodiments, the sample is taken from the subject (e.g., human subject) within about 2 hours of an actual or suspected injury to the head. For example, the sample can be taken from the subject within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, or about 2 hours of injury after an actual or suspected injury to the head. In some embodiments, the onset of the presence of the biomarker, such as UCH-L1, GFAP, or a combination thereof, appears within about 0 minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 20 minutes, about 30 minutes, about 60 minutes, about 90 minutes, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, within about 24 hours, within about 25 hours, within about 26 hours, within about 27 hours, within about 28 hours, within about 29 hours, within about 30 hours, within about 31 hours, within about 32 hours, within about 33 hours, within about 34 hours, within about 35 hours, within about 36 hours, within about 37 hours, within about 38 hours, within about 39 hours, within about 40 hours, within about 41 hours, within about 42 hours, within about 43 hours, within about 44 hours, within about 45 hours, within about 46 hours, within about 47 hours, or within about 48 hours after an actual or suspected injury to the head.

In other embodiments, the sample is taken within about 8 hours to within about 48 hours after the actual or suspected injury to the head. In still other embodiments, the sample is taken within about 9 hours to within about 48 hours after the actual or suspected injury to the head. In still other embodiments, the sample is taken within about 10 hours to within about 48 hours after the actual or suspected injury to the head. In still other embodiments, the sample is taken within about 11 hours to within about 48 hours after the actual or suspected injury to the head. In still other embodiments, the sample is taken within about 12 hours to within about 48 hours after the actual or suspected injury to the head. In still other embodiments, the sample is taken within about 13 hours to within about 48 hours after the actual or suspected injury to the head. In still other embodiments, the sample is taken within about 14 hours to within about 48 hours after the actual or suspected injury to the head. In still other embodiments, the sample is taken within about 15 hours to within about 48 hours after the actual or suspected injury to the head. In still other embodiments, the sample is taken within about 16 hours to within about 48 hours after the actual or suspected injury to the head. In still other embodiments, the sample is taken within about 17 hours to within about 48 hours after the actual or suspected injury to the head. In still other embodiments, the sample is taken within about 18 hours to within about 48 hours after the actual or suspected injury to the head. In still other embodiments, the sample is taken within about 19 hours to within about 48 hours after the actual or suspected injury to the head. In still other embodiments, the sample is taken within about 20 hours to within about 48 hours after the actual or suspected injury to the head. In still other embodiments, the sample is taken within about 21 hours to within about 48 hours after the actual or suspected injury to the head. In still other embodiments, the sample is taken within about 22 hours to within about 48 hours after the actual or suspected injury to the head. In still other embodiments, the sample is taken within about 23 hours to within about 48 hours after the actual or suspected injury to the head. In still other embodiments, the sample is taken within about 24 hours to within about 48 hours after the actual or suspected injury to the head. In still other embodiments, the sample is taken within about 25 hours to within about 48 hours after the actual or suspected injury to the head. In still other embodiments, the sample is taken within about 26 hours to within about 48 hours after the actual or suspected injury to the head. In still other embodiments, the sample is taken within about 27 hours to within about 48 hours after the actual or suspected injury to the head. In still other embodiments, the sample is taken within about 28 hours to within about 48 hours after the actual or suspected injury to the head. In still other embodiments, the sample is taken within about 29 hours to within about 48 hours after the actual or suspected injury to the head. In still other embodiments, the sample is taken within about 30 hours to within about 48 hours after the actual or suspected injury to the head. In still other embodiments, the sample is taken within about 31 hours to within about 48 hours after the actual or suspected injury to the head. In still other embodiments, the sample is taken within about 32 hours to within about 48 hours after the actual or suspected injury to the head. In still other embodiments, the sample is taken within about 33 hours to within about 48 hours after the actual or suspected injury to the head. In still other embodiments, the sample is taken within about 34 hours to within about 48 hours after the actual or suspected injury to the head. In still other embodiments, the sample is taken within about 35 hours to within about 48 hours after the actual or suspected injury to the head. In still other embodiments, the sample is taken within about 36 hours to within about 48 hours after the actual or suspected injury to the head. In still other embodiments, the sample is taken within about 37 hours to within about 48 hours after the actual or suspected injury to the head. In still other embodiments, the sample is taken within about 38 hours to within about 48 hours after the actual or suspected injury to the head. In still other embodiments, the sample is taken within about 39 hours to within about 48 hours after the actual or suspected injury to the head. In still other embodiments, the sample is taken within about 40 hours to within about 48 hours after the actual or suspected injury to the head.

In some embodiments, the subject has received a CT scan before or after the assay is performed. In some embodiments, the subject is suspected as having a traumatic brain injury based on the CT scan. In some embodiments, the reference level of the biomarker, such as UCH-L1, GFAP, or a combination thereof, is correlated with positive head CT scan.

Generally, a reference level of the biomarker, such as UCH-L1, GFAP, or a combination thereof, can be employed as a benchmark against which to assess results obtained upon assaying a test sample for UCH-L1, GFAP, or a combination thereof. Generally, in making such a comparison, the reference level of the biomarker, such as UCH-L1, GFAP, or a combination thereof, is obtained by running a particular assay a sufficient number of times and under appropriate conditions such that a linkage or association of analyte presence, amount or concentration with a particular stage or endpoint of TBI or with particular indicia can be made. Typically, the reference level of the biomarker, such as UCH-L1, GFAP, or a combination thereof, is obtained with assays of reference subjects (or populations of subjects). The biomarker, such as UCH-L1, GFAP, or a combination thereof, measured can include fragments thereof, degradation products thereof, and/or enzymatic cleavage products thereof.

In yet still further embodiments, the method comprises performing a head CT scan or an MRI on the subject when the subject's levels of the TBI biomarker (e.g., GFAP, UCH-L1, or GFAP and UCH-L1) are elevated. For example, in some embodiments the method comprises performing a head CT scan or a MRI procedure on the subject when the level of GFAP alone in the sample obtained from the subject is equal to or above the threshold value of about 30 μg/mL or 65 μg/mL, the level of GFAP and the level of UCH-L1 is below the threshold value of about 360 μg/mL, cannot be determined, or is not reported. In some embodiments, the method comprises performing a head CT scan or a MRI procedure on the subject when the level of UCH-L1 alone in the sample is equal to or above the threshold value of about 360 μg/mL, or level of GFAP in the sample obtained from the subject is equal to or above the threshold value of about 30 μg/mL or 65 μg/mL and the level of UCH-L1 in the sample is equal to or above the threshold value of about 360 μg/mL. In some embodiments, the method comprises performing a head CT scan or a MRI procedure on the subject when the level of GFAP in the sample obtained from the subject cannot be determined or is not reported and the level of UCH-L1 in the sample is equal to or above the threshold value of about 360 μg/mL.

In some embodiments, the method comprises determining that the subject does not require a head CT scan or an MRI when the subject's levels of the TBI biomarker (e.g., GFAP and/or UCH-L1) are not elevated. For example, in some embodiments the method comprises determining that the subject does not require a head CT scan or a MRI procedure when level of GFAP alone in the sample is below about 30 μg/mL or 65 μg/mL, the level of UCH-L1 alone in the sample is below about 360 μg/mL, or the level of GFAP in the sample obtained from the subject is below the threshold value of about 30 μg/mL or 65 μg/mL and when the level of UCH-L1 in the sample is below the threshold value of about 360 μg/mL.

In some embodiments, the method further includes treating the subject (e.g., human subject) with a traumatic brain injury treatment and/or monitoring the subject, as described below.

### 8. Apparatus, Non-Transitory Machine-Readable and Computer Readable Storage Mediums, and Systems

In some embodiments, disclosed herein are apparatus, machine-readable and computer readable storage mediums, and systems for use in implementing or performing the methods described in Sections 3-5. More specifically, the apparatus, machine-readable and computer readable storage mediums and systems described herein can be used to identify at least one biomarker in one or more biological samples obtained from a subject, determine the amount, concentration or level of one or more biomarkers in the one or more biological samples, and communicate from an apparatus (e.g., such as a point-of-care, non-point-of-care, or a point-of-care or non-point-of-care apparatus), whether the level of one or more biomarkers, is greater than, less than, or between one or more reference or other levels. As used herein, the term “apparatus” is used interchangeably with “device” or “instrument”.

In another embodiment, the present disclosure relates to an apparatus, device, or instrument. The apparatus, device, or instrument contains software to execute one or more tasks, including the performance of the methods described in Sections 3-5. In some embodiments, the apparatus, device, or instrument contains software to automatically determine the next appropriate step in the methods described herein. For example, the apparatus, device, or instrument may contain software that determines the concentration or level of a biomarker. The software may display this determination, such as on a graphical user interface.

In some embodiments, the apparatus, device, or instruments stores software that instructs processor or processor circuitry to execute or instantiate a given task. In some embodiments, the software stores machine-readable instructions that cause processor circuitry to execute or instantiate a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or non-transitory machine-readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term “non-transitory computer readable medium” is defined to include any type of computer readable storage device and/or storage disk and to exclude propagating signals and to exclude transmission media.

In some further embodiments, disclosed herein is a system for measuring or determining the amount or level of one or more biomarkers in a biological sample. The system comprises:
a. a data-obtaining module to obtain data of a concentration or level of at least one biomarker from one or more assays performed on at least one or more biological samples obtained from a subject, where a solution containing at least one nuclease is used when performing the steps of the assay;
b. an evaluation module to analyze the data to obtain an evaluation result of the amount or level of at least one biomarker in the biological sample (e.g., if the amount or level of one biomarker in the biological sample is being determined then a first evaluation result is provided; if the amount or level of two or more biomarkers in the biological sample is being determined then a first evaluation result is provided for the first biomarker and a second evaluation result is provided for the second biomarkers; or if the amount or level of three or more biomarkers in the biological is being determined then a first evaluation result is provided for the first biomarker, a second evaluation result is provided for the second biomarker, and a third evaluation result is provided for the third biomarker); and
c. a data-outputting module to output the evaluation result (e.g., the first evaluation results, the second evaluation result or the third evaluation result).

In some embodiments of the system, the at least one nuclease is an endonuclease. In still other embodiments, the at least one nuclease is a Benzonase® nuclease. Benzonase® nuclease is also known as a Serratia marcescens nuclease.

In some embodiments of the system, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In yet further embodiments, the solution contains about 1 U/mL to about 1.5 U/mL of the nuclease. In some embodiments, the solution comprising a nuclease does not contain heparin. In some embodiments, the system does not employ heparin.

In some embodiments of the system, the at least one biomarker is a biomarker of acquired brain injury. In other embodiments, the at least one biomarker is a biomarker of traumatic brain injury. In still further embodiments, the at least one biomarker is GFAP. In still further embodiments, the at least one biomarker is UCH-L1. In yet further embodiments, the at least one biomarker is GFAP and UCH-L1.

In further embodiments, disclosed herein is a computer device that comprises a storage device having machine-readable instructions stored thereon, and a processor. The processor executes the machine-readable instructions to perform the steps of the above computer-implemented method or the steps performed by the above evaluation system.

In further embodiments, disclosed herein is an apparatus, device, or instrument that comprises a storage device having machine-readable instructions stored thereon, and a processor. The processor instantiates or executes the machine-readable instructions to:
(A) identify at least one biomarker (e.g., a first biomarker, a second biomarker, a third biomarker, a fourth biomarker, or a fifth biomarker) in at least one or more biological samples obtained from a subject using at least one assay, where the at least one assay is performed using a solution containing at least one nuclease;
(B) determine an amount or level of at least one biomarker (e.g., a first biomarker, a second biomarker, a third biomarker, a fourth biomarker, or a fifth biomarker) in the one or more biological samples, determine a level of at least one biomarker (e.g., a first biomarker, a second biomarker, a third biomarker, a fourth biomarker, or a fifth biomarker) in one or more biological samples; and
(C) communicate from the apparatus, device, or instrument (e.g., such as displaying on the apparatus, device, or instrument) the amount or level of the at least one biomarker determined in the biological sample, or alternatively, whether the subject's level of the at least one biomarker is greater than, less than, or between a reference level for the sample biomarker (e.g., if the level of GFAP is being determined, then a reference level for GFAP is used; if the level of UCH-L1 is being determined, then a reference level for UCH-L1 is used).

In some embodiments of the device, the at least one nuclease is an endonuclease. In still other embodiments of the device, the at least one nuclease is a Benzonase® nuclease.

In some embodiments of the apparatus, device, or instrument, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In yet further embodiments, the solution contains about 1 U/mL to about 1.5 U/mL of the nuclease. In some embodiments, the solution comprising a nuclease does not contain heparin. In some embodiments, no heparin is used in the device. In some embodiments, the solution comprising a nuclease further comprises one or more salts, such as magnesium chloride.

In some embodiments of the apparatus, device, or instrument, the at least one biomarker is a biomarker of acquired brain injury. In other embodiments, the at least one biomarker is a biomarker of traumatic brain injury. In still further embodiments, the at least one biomarker is GFAP. In still further embodiments the at least one biomarker is UCH-L1. In yet further embodiments, the at least one biomarker is GFAP and UCH-L1.

In further embodiments, disclosed herein is a non-transitory machine-readable storage medium having machine-readable instructions stored thereon, wherein the machine-readable instructions are configured to be executed by a processor to perform the steps of the above described computer-implemented method or the steps performed by the above one or more systems, such as the evaluation system.

In yet further embodiments, disclosed herein is a non-transitory machine-readable storage medium. The non-transitory machine-readable storage medium can comprise instructions to cause one or processors or processor circuitry to at least:
(A) identify at least one biomarker (e.g., a first biomarker, a second biomarker, a third biomarker, a fourth biomarker, a fifth biomarker), in at least one or more biological samples obtained from a subject using at least one assay, where the at least one assay is performed using a solution containing at least one nuclease;
(B) determine an amount or level of at least one biomarker in the one or more biological samples, determine a level on at least one biomarker (e.g., (e.g., a first biomarker, a second biomarker, a third biomarker, a fourth biomarker, a fifth biomarker), in one or more biological samples; and
(C) communicate from an apparatus, device, or instrument (e.g., such as displaying on the apparatus, device, or instrument) whether the subject's level of the at least one biomarker (e.g., a first biomarker, a second biomarker, a third biomarker, a fourth biomarker, a fifth biomarker).

In some embodiments of the non-transitory machine-readable storage medium, the at least one biomarker is a biomarker of acquired brain injury. In other embodiments, the at least one biomarker is a biomarker of traumatic brain injury. In yet further embodiments, the at least one biomarker is GFAP. In still further embodiments, the biomarker is UCH-L1. In yet still further embodiments, the biomarker is GFAP and UCH-L1.

In some embodiments of the non-transitory machine-readable storage medium the at least one nuclease is an endonuclease. In still other embodiments of the non-transitory machine-readable storage medium at least one nuclease is a Benzonase® nuclease.

In some embodiments of the non-transitory machine-readable storage medium, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In yet further embodiments, the solution contains about 1 U/mL to about 1.5 U/mL of the nuclease. In some embodiments, the solution comprising a nuclease does not contain heparin. In some embodiments, the non-transitory machine-readable storage medium does not employ any heparin.

In still further embodiments, disclosed herein is a computer readable storage medium having stored thereon a computer program, where the computer program implements steps of a method of measuring an amount or level of at least one biomarker in a sample when executed by a processor, where the method comprises:
a) acquiring signal data generated by contacting a sample obtained from a subject, either simultaneously or sequentially, in any order, with: (1) one or more capture antibodies which bind to an epitope on the at least one biomarker to form a capture antibody-biomarker-complex; (2) one or more detection antibodies which bind to an epitope on the biomarker that is not bound by the capture antibody to form a capture antibody-biomarker-detection antibody complex; and (3) a solution comprising at least one nuclease; and
b) calculating an amount or level of the biomarker in the sample based upon the data from step a).

In some embodiments of the computer readable storage medium, the at least one biomarker is a biomarker of acquired brain injury. In other embodiments, the at least one biomarker is a biomarker of traumatic brain injury. In yet further embodiments, the at least one biomarker is GFAP. In still further embodiments, the biomarker is UCH-L1. In yet still further embodiments, the biomarker is GFAP and UCH-L1.

In some embodiments of computer readable storage medium, the at least one nuclease is an endonuclease. In still other embodiments of the non-transitory machine-readable storage medium at least one nuclease is a Benzonase® nuclease.

In some embodiments computer readable storage medium, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In yet further embodiments, the solution contains about 1 U/mL to about 1.5 U/mL of the nuclease. In some embodiments, the solution comprising a nuclease does not contain heparin. In some embodiments, the solution comprising a nuclease further comprises one or more salts. In yet further embodiments, the salt is magnesium chloride.

In yet further embodiments, disclosed herein is computer readable storage medium having stored thereon a computer program, where the computer program implements steps of a method of an assay for measuring an amount or level of at least one biomarker in a sample when executed by a processor, wherein the method comprises the steps of:
a) acquiring signal data generated by a solution comprising at least one nuclease when performing the assay; and
b) calculating an amount or level of the at least one biomarker in the sample based upon the data from step a).

In some embodiments of the computer readable storage medium, the at least one biomarker is a biomarker of acquired brain injury. In other embodiments, the at least one biomarker is a biomarker of traumatic brain injury. In yet further embodiments, the at least one biomarker is GFAP. In still further embodiments, the biomarker is UCH-L1. In yet still further embodiments, the biomarker is GFAP and UCH-L1.

In some embodiments of computer readable storage medium, the at least one nuclease is an endonuclease. In still other embodiments of the non-transitory machine-readable storage medium at least one nuclease is a Benzonase® nuclease.

In some embodiments computer readable storage medium, the solution comprising a nuclease comprises at least about 1 U/mL nuclease (e.g., about 1 U/mL nuclease, about 1.1 U/mL nuclease, about 1.2 U/mL nuclease, about 1.3 U/mL nuclease, about 1.4 U/mL nuclease, about 1.5 U/mL nuclease, about 1.6 U/mL nuclease, about 1.7 U/mL nuclease, about 1.8 U/mL nuclease, about 1.9 U/mL nuclease, or about 2.0 U/mL nuclease). In yet further embodiments, the solution contains about 1 U/mL to about 1.5 U/mL of the nuclease. In some embodiments, the solution comprising a nuclease does not contain heparin. In yet further embodiments, the solution further comprises at least one salt. In still further embodiments, the at least one salt is magnesium chloride.

### 9. Treatment and Monitoring of a Subject Suffering from an Acquired Brain Injury

The subject (e.g., a human subject) identified or assessed in the methods described above as having elevated levels of an ABI biomarker, such as a TBI biomarker (e.g., GFAP, UCH-L1, or GFAP and UCH-L1), which may be indicative of an acquired brain injury, such as a traumatic brain injury, may be treated or monitored. In some embodiments, the method further includes treating the subject (e.g., human subject) determined as having elevated levels of GFAP and/or UCH-L1 with a traumatic brain injury treatment, such as any treatments known in the art. For example, treatment of traumatic brain injury can take a variety of forms depending on the severity of the injury to the head. For example, for subjects suffering from mild TBI, the treatment may include one or more of rest, abstaining for physical activities, such as sports, avoiding light or wearing sunglasses when out in the light, medication for relief of a headache or migraine, anti-nausea medication, etc. Treatment for patients suffering from moderate, severe, or moderate to severe TBI might include administration of one or more appropriate medications (such as, for example, diuretics, anti-convulsant medications, medications to sedate and put an individual in a drug-induced coma, or other pharmaceutical or biopharmaceutical medications (either known or developed in the future for treatment of TBI), one or more surgical procedures (such as, for example, removal of a hematoma, repairing a skull fracture, decompressive craniectomy, etc.) and one or more therapies (such as, for example one or more rehabilitation (e.g., such as physical therapy, occupational therapy, or combinations thereof), cognitive behavioral therapy, anger management, counseling psychology, etc.). In some embodiments, the method further includes monitoring the subject (e.g., a human subject) assessed as having elevated levels of the TBI biomarker. For example, in some embodiments the method further includes monitoring the subject assessed as having elevated levels of GFAP, UCH-L1, or GFAP and UCH-L1 (e.g., which may be indicative or mild, moderate, severe, or moderate to severe traumatic brain injury, or mild, moderate, severe, or moderate to severe traumatic brain injury). For example, monitoring the subject assessed as having elevated levels of GFAP, UCH-L1, or GFAP and UCH-L1 may comprise monitoring with a CT scan and/or a MRI procedure. In some embodiments, a subject identified as having traumatic brain injury, such as mild traumatic brain injury, moderate traumatic brain injury, severe traumatic brain injury, or moderate to severe traumatic brain injury or mild traumatic brain injury, moderate traumatic brain injury, severe traumatic brain injury, or moderate to severe traumatic brain injury may be monitored with CT scan and/or MRI.

### 10. Methods for Measuring the Level of UCH-L1

In some embodiments, measuring the level of UCH-L1 includes contacting the sample with a first specific binding member and second specific binding member. In some embodiments the first specific binding member is a capture antibody and the second specific binding member is a detection antibody. In some embodiments, measuring the level of UCH-L1 includes contacting the sample, either simultaneously or sequentially, in any order, with: (1) a capture antibody (e.g., UCH-L1-capture antibody), which binds to an epitope on UCH-L1 or UCH-L1 fragment to form a capture antibody-UCH-L1 antigen complex (e.g., UCH-L1-capture antibody-UCH-L1 antigen complex), and (2) a detection antibody (e.g., UCH-L1-detection antibody), which includes a detectable label and binds to an epitope on UCH-L1 that is not bound by the capture antibody, to form a UCH-L1 antigen-detection antibody complex (e.g., UCH-L1 antigen-UCH-L1-detection antibody complex), such that a capture antibody-UCH-L1 antigen-detection antibody complex (e.g., UCH-L1-capture antibody-UCH-L1 antigen-UCH-L1-detection antibody complex) is formed, and measuring the amount or concentration of UCH-L1 in the sample based on the signal generated by the detectable label in the capture antibody-UCH-L1 antigen-detection antibody complex.

In some embodiments, the first specific binding member is immobilized on a solid support. In some embodiments, the second specific binding member is immobilized on a solid support. In some embodiments, the first specific binding member is a UCH-L1 antibody as described below.

In some embodiments, the sample is diluted or undiluted. In some embodiments, the sample is about 1 to about 30 microliters. In some embodiments, the sample is about 10 to about 30 microliters. In some embodiments, the sample is about 20 microliters. In some embodiments, the sample is from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, or about 15 microliters. In some embodiments, the sample is about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters, about 25 microliters, about 26 microliters, about 27 microliters, about 28 microliters, about 29 microliters, or about 30 microliters. In some embodiments, the sample is from about 1 to about 150 microliters or less or from about 1 to about 30 microliters or less.

Some instruments (such as, for example the Abbott Laboratories instruments ARCHITECT®, Alinity, and other core laboratory instruments) other than a point-of-care device may be capable of measuring levels of UCH-L1 in a sample higher or greater than 25,000 μg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 11. UCH-L1 Antibodies

The methods described herein may use an isolated antibody that specifically binds to ubiquitin carboxy-terminal hydrolase L1 (“UCH-L1”) (or fragments thereof), referred to as “UCH-L1 antibody.” The UCH-L1 antibodies can be used to assess the UCH-L1 status as a measure of traumatic brain injury, detect the presence of UCH-L1 in a sample, quantify the amount of UCH-L1 present in a sample, or detect the presence of and quantify the amount of UCH-L1 in a sample.

a. Ubiquitin Carboxy-Terminal Hydrolase L1 (UCH-L1)

Ubiquitin carboxy-terminal hydrolase L1 (“UCH-L1”), which is also known as “ubiquitin C-terminal hydrolase,” is a deubiquitinating enzyme. UCH-L1 is a member of a gene family whose products hydrolyze small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary. The catalytic triad of UCH-L1 contains a cysteine at position 90, an aspartate at position 176, and a histidine at position 161 that are responsible for its hydrolase activity.

Human UCH-L1 may have the following amino acid sequence:

(SEQ ID NO: 1)
MQLKPMEINPEMLNKVLSRLGVAGQWRFVDVLGLEEESLGSVPAPACALL
LLFPLTAQHENFRKKQIEELKGQEVSPKVYFMKQTIGNSCGTIGLIHAVA
NNQDKLGFEDGSVLKQFLSETEKMSPEDRAKCFEKNEAIQAAHDAVAQEG
QCRVDDKVNFHFILFNNVDGHLYELDGRMPFPVNHGASSEDTLLKDAAKV
CREFTEREQGEVRESAVALCKAA.

The human UCH-L1 may be a fragment or variant of SEQ ID NO: 1. The fragment of UCH-L1 may be between 5 and 225 amino acids, between 10 and 225 amino acids, between 50 and 225 amino acids, between 60 and 225 amino acids, between 65 and 225 amino acids, between 100 and 225 amino acids, between 150 and 225 amino acids, between 100 and 175 amino acids, or between 175 and 225 amino acids in length. The fragment may comprise a contiguous number of amino acids from SEQ ID NO: 1.

b. UCH-L1-Recognizing Antibody

The antibody is an antibody that binds to UCH-L1, a fragment thereof, an epitope of UCH-L1, or a variant thereof. The antibody may be a fragment of the anti-UCH-L1 antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-UCH-L1 antibodies may be a chimeric anti-UCH-L1 or humanized anti-UCH-L1 antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-UCH-L1 antibodies. For example, libraries of naïve human single chain variable fragments (scFv) may be used to select human anti-UCH-L1 antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to UCH-L1 (SEQ ID NO: 1), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region. In some embodiments, the epitope has a length of between 3 to 25 amino acids, between about 5 to 25 amino acids, between 5 to 20 amino acids, between 5 to 15 amino acids, between 10 to 25 amino acids, between 10 to 20 amino acids, or between 10 to 15 amino acids.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human UCH-L1) and the other heavy and light chain are specific for an antigen other than human UCH-L1 by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with UCH-L1 or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al., J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. Sec, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two fold stronger than the binding affinity of the parent antibody for the antigen.

The antibody may be a linear antibody. The procedure for making a linear antibody is known in the art and described in Zapata et al., (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (1311), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-UCH-L1 Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with UCH-L1 with myeloma cells and then screening the hybridomas resulting from the fusion for hybridoma clones that secrete an antibody able to bind a polypeptide of the disclosure. Briefly, rats can be immunized with a UCH-L1 antigen. In a preferred embodiment, the UCH-L1 antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a UCH-L1 antigen, antibodies and/or antibody-producing cells may be obtained from the animal. An anti-UCH-L1 antibody-containing serum is obtained from the animal by bleeding or sacrificing the animal. The serum may be used as it is obtained from the animal, an immunoglobulin fraction may be obtained from the serum, or the anti-UCH-L1 antibodies may be purified from the serum. Serum or immunoglobulins obtained in this manner are polyclonal, thus having a heterogeneous array of properties.

Once an immune response is detected, e.g., antibodies specific for the antigen UCH-L1 are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding UCH-L1. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. Sec, e.g., Harlow and Lane, supra. In a preferred embodiment, the myeloma cells do not secrete immunoglobulin polypeptides (a non-secretory cell line). After fusion and antibiotic selection, the hybridomas are screened using UCH-L1, or a portion thereof, or a cell expressing UCH-L1. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-UCH-L1 antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-UCH-L1 antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-UCH-L1 Monoclonal Antibodies Using SLAM

In another embodiment of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen UCH-L1, a subunit of UCH-L1, or a fragment thereof, is coupled to sheep red blood cells using a linker, such as biotin, and used to identify single cells that secrete antibodies with specificity for UCH-L1. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to UCH-L1. The amplified immunoglobulin sequences further can be manipulated in vitro, such as by in vitro affinity maturation method. See, for example, PCT Publication No. WO 97/29131 and PCT Publication No. WO 00/56772.

### (3) Anti-UCH-L1 Monoclonal Antibodies Using Transgenic Animals

In another embodiment of the disclosure, antibodies are produced by immunizing a non-human animal comprising some, or all, of the human immunoglobulin locus with a UCH-L1 antigen. In an embodiment, the non-human animal is a XENOMOUSE® transgenic mouse, an engineered mouse strain that comprises large fragments of the human immunoglobulin loci and is deficient in mouse antibody production. Sec, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. Sec also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

### (4) Anti-UCH-L1 Monoclonal Antibodies Using Recombinant Antibody Libraries

In vitro methods also can be used to make the antibodies of the disclosure, wherein an antibody library is screened to identify an antibody having the desired UCH-L1-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with UCH-L1, or a portion of UCH-L1. Alternatively, the recombinant antibody library may be from a naive subject, i.e., one who has not been immunized with UCH-L1, such as a human antibody library from a human subject who has not been immunized with human UCH-L1. Antibodies of the disclosure are selected by screening the recombinant antibody library with the peptide comprising human UCH-L1 to thereby select those antibodies that recognize UCH-L1. Methods for conducting such screening and selection are well known in the art, such as described in the references in the preceding paragraph. To select antibodies of the disclosure having particular binding affinities for UCH-L1, such as those that dissociate from human UCH-L1 with a particular K_{off} rate constant, the art-known method of surface plasmon resonance can be used to select antibodies having the desired K_{off} rate constant. To select antibodies of the disclosure having a particular neutralizing activity for hUCH-L1, such as those with a particular IC₅₀, standard methods known in the art for assessing the inhibition of UCH-L1 activity may be used.

In one embodiment, the disclosure pertains to an isolated antibody, or an antigen-binding portion thereof, that binds human UCH-L1. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant UCH-L1 Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human UCH-L1) and the other heavy and light chain are specific for an antigen other than human UCH-L1 by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for UCH-L1 and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for UCH-L1, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g., mice that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-UCH-L1 Antibodies

Anti-UCH-L1 antibodies may be generated using the techniques described above as well as using routine techniques known in the art. In some embodiments, the anti-UCH-L1 antibody may be an unconjugated UCH-L1 antibody, such as UCH-L1 antibodies available from United State Biological (Catalog Number: 031320), Cell Signaling Technology (Catalog Number: 3524), Sigma-Aldrich (Catalog Number: HPA005993), Santa Cruz Biotechnology, Inc. (Catalog Numbers: sc-58593 or sc-58594), R&D Systems (Catalog Number: MAB6007), Novus Biologicals (Catalog Number: NB600-1160), Biorbyt (Catalog Number: orb33715), Enzo Life Sciences, Inc. (Catalog Number: ADI-905-520-1), Bio-Rad (Catalog Number: VMA00004), Bio Vision (Catalog Number: 6130-50), Abcam (Catalog Numbers: ab75275 or ab104938), Invitrogen Antibodies (Catalog Numbers: 480012), ThermoFisher Scientific (Catalog Numbers: MA1-46079. MA5-17235. MA1-90008, or MA1-83428), EMD Millipore (Catalog Number: MABN48), or Sino Biological Inc. (Catalog Number: 50690-R011). The anti-UCH-L1 antibody may be conjugated to a fluorophore, such as conjugated UCH-L1 antibodies available from BioVision (Catalog Number: 6960-25) or Aviva Systems Biology (Cat. Nos. OAAF01904-FITC).

Alternatively, the antibodies described in WO 2018/067468 and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 12. Methods for Measuring the Level of GFAP

In the methods described above, GFAP levels can be measured by any means, such as antibody dependent methods, such as immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in clinical chemistry format such as would be known by one skilled in the art.

In some embodiments, measuring the level of GFAP includes contacting the sample with a first specific binding member and second specific binding member. In some embodiments the first specific binding member is a capture antibody and the second specific binding member is a detection antibody. In some embodiments, measuring the level of GFAP includes contacting the sample, either simultaneously or sequentially, in any order, with: (1) a capture antibody (e.g., GFAP-capture antibody), which binds to an epitope on GFAP or GFAP fragment to form a capture antibody-GFAP antigen complex (e.g., GFAP-capture antibody-GFAP antigen complex), and (2) a detection antibody (e.g., GFAP-detection antibody), which includes a detectable label and binds to an epitope on GFAP that is not bound by the capture antibody, to form a GFAP antigen-detection antibody complex (e.g., GFAP antigen-GFAP-detection antibody complex), such that capture antibody-GFAP antigen-detection antibody complex (e.g., GFAP-capture antibody-GFAP antigen-GFAP-detection antibody complex) is formed, and measuring the amount or concentration of GFAP in the sample based on the signal generated by the detectable label in the capture antibody-GFAP antigen-detection antibody complex.

In some embodiments, the first specific binding member is immobilized on a solid support. In some embodiments, the second specific binding member is immobilized on a solid support. In some embodiments, the first specific binding member is a GFAP antibody as described below.

In some embodiments, the sample is diluted or undiluted. In some embodiments, the sample is about 1 to about 30 microliters. In some embodiments, the sample is about 10 to about 30 microliters. In some embodiments, the sample is about 20 microliters. In some embodiments, the sample is from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, or about 15 microliters. In some embodiments, the sample is about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters about 25 microliters, about 26 microliters, about 27 microliters, about 28 microliters, about 29 microliters, or about 30 microliters. In some embodiments, the sample is from about 1 to about 150 microliters or less or from about 1 to about 30 microliters or less.

Some instruments (such as, for example the Abbott Laboratories instruments ARCHITECT, Alinity, and other core laboratory instruments) other than a point-of-care device may be capable of measuring levels of GFAP in a sample higher or greater than 25,000 μg/mL.

Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety

### 13. GFAP Antibodies

The methods described herein may use an isolated antibody that specifically binds to Glial fibrillary acidic protein (“GFAP”) (or fragments thereof), referred to as “GFAP antibody.” The GFAP antibodies can be used to assess the GFAP status as a measure of traumatic brain injury, detect the presence of GFAP in a sample, quantify the amount of GFAP present in a sample, or detect the presence of and quantify the amount of GFAP in a sample.

a. Glial Fibrillary Acidic Protein (GFAP)

Glial fibrillary acidic protein (GFAP) is a 50 kDa intracytoplasmic filamentous protein that constitutes a portion of the cytoskeleton in astrocytes, and it has proved to be the most specific marker for cells of astrocytic origin. GFAP protein is encoded by the GFAP gene in humans. GFAP is the principal intermediate filament of mature astrocytes. In the central rod domain of the molecule, GFAP shares considerable structural homology with the other intermediate filaments. GFAP is involved in astrocyte motility and shape by providing structural stability to astrocytic processes. Glial fibrillary acidic protein and its breakdown products (GFAP-BDP) are brain-specific proteins released into the blood as part of the pathophysiological response after traumatic brain injury (TBI). Following injury to the human CNS caused by trauma, genetic disorders, or chemicals, astrocytes proliferate and show extensive hypertrophy of the cell body and processes, and GFAP is markedly upregulated. In contrast, with increasing astrocyte malignancy, there is a progressive loss of GFAP production. GFAP can also be detected in Schwann cells, enteric glia cells, salivary gland neoplasms, metastasizing renal carcinomas, epiglottic cartilage, pituicytes, immature oligodendrocytes, papillary meningiomas, and myoepithelial cells of the breast.

Human GFAP may have the following amino acid sequence:

(SEQ ID NO: 2)
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALARLEEEGQSLKDEMARHLQEYQ
DLLNVKLALDIEIATYRKLLEGEENRITIPVQTFSNLQIRETSLDTKSVS
EGHLKRNIVVKTVEMRDGEVIKESKQEHKDVM.

The human GFAP may be a fragment or variant of SEQ ID NO: 2. The fragment of GFAP may be between 5 and 400 amino acids, between 5 and 300 amino acids, between 5 and 200 amino acids, between 5 and 100 amino acids, between 5 and 50 amino acids, between 5 and 25 amino acids, between 5 and 15 amino acids, between 10 and 400 amino acids, between 10 and 300 amino acids, between 10 and 200 amino acids, between 10 and 100 amino acids, between 10 and 50 amino acids, between 10 and 25 amino acids, between 50 and 400 amino acids, between 60 and 400 amino acids, between 65 and 400 amino acids, between 100 and 400 amino acids, between 150 and 400 amino acids, between 100 and 300 amino acids, or between 200 and 300 amino acids in length. The fragment may comprise a contiguous number of amino acids from SEQ ID NO: 2. The human GFAP fragment or variant of SEQ ID NO: 2 may be a GFAP breakdown product (BDP). The GFAP BDP may be 38 kDa, 42 kDa (fainter 41 kDa), 47 kDa (fainter 45 kDa); 25 kDa (fainter 23 kDa); 19 kDa, or 20 kDa. In some embodiments, the human GFAP fragment or variant can be a GFAP BDP comprising between 5 to 25 amino acids, between 5 to 50 amino acids, between 5 to 100 amino acids, 5 to 200 amino acids, between 10 to 20 amino acids, between 10 to 25 amino acids, between 10 to 50 amino acids, between 10 to 100 amino acids, or between 10 to 200 amino acids.

b. GFAP-Recognizing Antibody

The antibody is an antibody that binds to GFAP, a fragment thereof, an epitope of GFAP, or a variant thereof. The antibody may be a fragment of the anti-GFAP antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

The anti-GFAP antibodies may be a chimeric anti-GFAP or humanized anti-GFAP antibody. In one embodiment, both the humanized antibody and chimeric antibody are monovalent. In one embodiment, both the humanized antibody and chimeric antibody comprise a single Fab region linked to an Fc region.

Human antibodies may be derived from phage-display technology or from transgenic mice that express human immunoglobulin genes. The human antibody may be generated as a result of a human in vivo immune response and isolated. See, for example, Funaro et al., BMC Biotechnology, 2008 (8): 85. Therefore, the antibody may be a product of the human and not animal repertoire. Because it is of human origin, the risks of reactivity against self-antigens may be minimized. Alternatively, standard yeast display libraries and display technologies may be used to select and isolate human anti-GFAP antibodies. For example, libraries of naïve human single chain variable fragments (scFv) may be used to select human anti-GFAP antibodies. Transgenic animals may be used to express human antibodies.

Humanized antibodies may be antibody molecules from non-human species antibody that binds the desired antigen having one or more complementarity determining regions (CDRs) from the non-human species and framework regions from a human immunoglobulin molecule.

The antibody is distinguishable from known antibodies in that it possesses different biological function(s) than those known in the art.

### (1) Epitope

The antibody may immunospecifically bind to GFAP (SEQ ID NO: 2), a fragment thereof, or a variant thereof. The antibody may immunospecifically recognize and bind at least three amino acids, at least four amino acids, at least five amino acids, at least six amino acids, at least seven amino acids, at least eight amino acids, at least nine amino acids, or at least ten amino acids within an epitope region. The antibody may immunospecifically recognize and bind to an epitope that has at least three contiguous amino acids, at least four contiguous amino acids, at least five contiguous amino acids, at least six contiguous amino acids, at least seven contiguous amino acids, at least eight contiguous amino acids, at least nine contiguous amino acids, or at least ten contiguous amino acids of an epitope region. In some embodiments, the epitope has a length of between 3 to 25 amino acids, between about 5 to 25 amino acids, between 5 to 20 amino acids, between 5 to 15 amino acids, between 10 to 25 amino acids, between 10 to 20 amino acids, or between 10 to 15 amino acids.

c. Antibody Preparation/Production

Antibodies may be prepared by any of a variety of techniques, including those well known to those skilled in the art. In general, antibodies can be produced by cell culture techniques, including the generation of monoclonal antibodies via conventional techniques, or via transfection of antibody genes, heavy chains, and/or light chains into suitable bacterial or mammalian cell hosts, in order to allow for the production of antibodies, wherein the antibodies may be recombinant. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies include Chinese Hamster Ovary (CHO cells) (including dhfr-CHO cells, described in Urlaub and Chasin, Proc. Natl. Acad. Sci. USA, 77:4216-4220 (1980)), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies. In addition, bifunctional antibodies may be produced in which one heavy and one light chain are an antibody (i.e., binds human GFAP) and the other heavy and light chain are specific for an antigen other than human GFAP by crosslinking an antibody to a second antibody by standard chemical crosslinking methods.

In a preferred system for recombinant expression of an antibody, or antigen-binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the method of synthesizing a recombinant antibody may be by culturing a host cell in a suitable culture medium until a recombinant antibody is synthesized. The method can further comprise isolating the recombinant antibody from the culture medium.

Methods of preparing monoclonal antibodies involve the preparation of immortal cell lines capable of producing antibodies having the desired specificity. Such cell lines may be produced from spleen cells obtained from an immunized animal. The animal may be immunized with GFAP or a fragment and/or variant thereof. The peptide used to immunize the animal may comprise amino acids encoding human Fc, for example the fragment crystallizable region or tail region of human antibody. The spleen cells may then be immortalized by, for example, fusion with a myeloma cell fusion partner. A variety of fusion techniques may be employed. For example, the spleen cells and myeloma cells may be combined with a nonionic detergent for a few minutes and then plated at low density on a selective medium that supports that growth of hybrid cells, but not myeloma cells. One such technique uses hypoxanthine, aminopterin, thymidine (HAT) selection. Another technique includes electrofusion. After a sufficient time, usually about 1 to 2 weeks, colonies of hybrids are observed. Single colonies are selected and their culture supernatants tested for binding activity against the polypeptide. Hybridomas having high reactivity and specificity may be used.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. Affinity chromatography is an example of a method that can be used in a process to purify the antibodies.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites.

The Fv fragment can be produced by preferential proteolytic cleavage of an IgM, and on rare occasions IgG or IgA immunoglobulin molecules. The Fv fragment may be derived using recombinant techniques. The Fv fragment includes a non-covalent VH::VL heterodimer including an antigen-binding site that retains much of the antigen recognition and binding capabilities of the native antibody molecule.

The antibody, antibody fragment, or derivative may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, yeast or the like, display library); e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161) that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al. (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994)).

An affinity matured antibody may be produced by any one of a number of procedures that are known in the art. For example, see Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); Hawkins et al, J. Mol. Biol., 226:889-896 (1992). Selective mutation at selective mutagenesis positions and at contact or hypermutation positions with an activity enhancing amino acid residue is described in U.S. Pat. No. 6,914,128 B1.

Antibody variants can also be prepared using delivering a polynucleotide encoding an antibody to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

Antibody variants also can be prepared by delivering a polynucleotide to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbiol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. Sec, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFvs), including tobacco seeds and potato tubers. Sec, e.g., Conrad et al. (1998) Plant Mol. Biol. 38:101-109 and reference cited therein. Thus, antibodies can also be produced using transgenic plants, according to known methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Small antibody fragments may be diabodies having two antigen-binding sites, wherein fragments comprise a heavy chain variable domain (VH) connected to a light chain variable domain (VL) in the same polypeptide chain (VH VL). See for example, EP 404,097; WO 93/11161; and Hollinger et al., (1993) Proc. Natl. Acad. Sci. USA 90:6444-6448. By using a linker that is too short to allow pairing between the two domains on the same chain, the domains are forced to pair with the complementary domains of another chain and create two antigen-binding sites. See also, U.S. Pat. No. 6,632,926 to Chen et al. which is hereby incorporated by reference in its entirety and discloses antibody variants that have one or more amino acids inserted into a hypervariable region of the parent antibody and a binding affinity for a target antigen which is at least about two-fold stronger than the binding affinity of the parent antibody for the antigen.

The antibody may be a linear antibody. The procedure for making a linear antibody is known in the art and described in Zapata et al. (1995) Protein Eng. 8 (10): 1057-1062. Briefly, these antibodies comprise a pair of tandem Fd segments (VH-CH1-VH-CH1) which form a pair of antigen binding regions. Linear antibodies can be bispecific or monospecific.

The antibodies may be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

It may be useful to detectably label the antibody. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (1311), yttrium-90 (90Y), bismuth-212 (212Bi), bismuth-213 (213Bi), technetium-99m (99mTc), rhenium-186 (186Re), and rhenium-188 (188Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibody production via the use of hybridoma technology, the selected lymphocyte antibody method (SLAM), transgenic animals, and recombinant antibody libraries is described in more detail below.

### (1) Anti-GFAP Monoclonal Antibodies Using Hybridoma Technology

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, second edition, (Cold Spring Harbor Laboratory Press, Cold Spring Harbor, 1988); Hammerling, et al., In Monoclonal Antibodies and T-Cell Hybridomas, (Elsevier, N.Y., 1981). It is also noted that the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods of generating monoclonal antibodies as well as antibodies produced by the method may comprise culturing a hybridoma cell secreting an antibody of the disclosure wherein, preferably, the hybridoma is generated by fusing splenocytes isolated from an animal, e.g., a rat or a mouse, immunized with GFAP with myeloma cells and then screening the hybridomas resulting from the fusion for hybridoma clones that secrete an antibody able to bind a polypeptide of the disclosure. Briefly, rats can be immunized with a GFAP antigen. In a preferred embodiment, the GFAP antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks; however, a single administration of the polypeptide may also be used.

After immunization of an animal with a GFAP antigen, antibodies and/or antibody-producing cells may be obtained from the animal. An anti-GFAP antibody-containing serum is obtained from the animal by bleeding or sacrificing the animal. The serum may be used as it is obtained from the animal, an immunoglobulin fraction may be obtained from the serum, or the anti-GFAP antibodies may be purified from the serum. Serum or immunoglobulins obtained in this manner are polyclonal, thus having a heterogeneous array of properties.

Once an immune response is detected, e.g., antibodies specific for the antigen GFAP are detected in the rat serum, the rat spleen is harvested and splenocytes isolated. The splenocytes are then fused by well-known techniques to any suitable myeloma cells, for example, cells from cell line SP20 available from the American Type Culture Collection (ATCC, Manassas, Va., US). Hybridomas are selected and cloned by limited dilution. The hybridoma clones are then assayed by methods known in the art for cells that secrete antibodies capable of binding GFAP. Ascites fluid, which generally contains high levels of antibodies, can be generated by immunizing rats with positive hybridoma clones.

In another embodiment, antibody-producing immortalized hybridomas may be prepared from the immunized animal. After immunization, the animal is sacrificed and the splenic B cells are fused to immortalized myeloma cells as is well known in the art. Sec, e.g., Harlow and Lane, supra. In a preferred embodiment, the myeloma cells do not secrete immunoglobulin polypeptides (a non-secretory cell line). After fusion and antibiotic selection, the hybridomas are screened using GFAP, or a portion thereof, or a cell expressing GFAP. In a preferred embodiment, the initial screening is performed using an enzyme-linked immunosorbent assay (ELISA) or a radioimmunoassay (RIA), preferably an ELISA. An example of ELISA screening is provided in PCT Publication No. WO 00/37504.

Anti-GFAP antibody-producing hybridomas are selected, cloned, and further screened for desirable characteristics, including robust hybridoma growth, high antibody production, and desirable antibody characteristics. Hybridomas may be cultured and expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, hybridomas are rat hybridomas. In another embodiment, hybridomas are produced in a non-human, non-rat species such as mice, sheep, pigs, goats, cattle, or horses. In yet another preferred embodiment, the hybridomas are human hybridomas, in which a human non-secretory myeloma is fused with a human cell expressing an anti-GFAP antibody.

Antibody fragments that recognize specific epitopes may be generated by known techniques. For example, Fab and F(ab′)₂ fragments of the disclosure may be produced by proteolytic cleavage of immunoglobulin molecules, using enzymes such as papain (to produce two identical Fab fragments) or pepsin (to produce an F(ab′)₂ fragment). A F(ab′)₂ fragment of an IgG molecule retains the two antigen-binding sites of the larger (“parent”) IgG molecule, including both light chains (containing the variable light chain and constant light chain regions), the CH1 domains of the heavy chains, and a disulfide-forming hinge region of the parent IgG molecule. Accordingly, an F(ab′)₂ fragment is still capable of crosslinking antigen molecules like the parent IgG molecule.

### (2) Anti-GFAP Monoclonal Antibodies Using SLAM

In another embodiment of the disclosure, recombinant antibodies are generated from single, isolated lymphocytes using a procedure referred to in the art as the selected lymphocyte antibody method (SLAM), as described in U.S. Pat. No. 5,627,052; PCT Publication No. WO 92/02551; and Babcook et al., Proc. Natl. Acad. Sci. USA, 93:7843-7848 (1996). In this method, single cells secreting antibodies of interest, e.g., lymphocytes derived from any one of the immunized animals are screened using an antigen-specific hemolytic plaque assay, wherein the antigen GFAP, a subunit of GFAP, or a fragment thereof, is coupled to sheep red blood cells using a linker, such as biotin, and used to identify single cells that secrete antibodies with specificity for GFAP. Following identification of antibody-secreting cells of interest, heavy- and light-chain variable region cDNAs are rescued from the cells by reverse transcriptase-PCR (RT-PCR) and these variable regions can then be expressed, in the context of appropriate immunoglobulin constant regions (e.g., human constant regions), in mammalian host cells, such as COS or CHO cells. The host cells transfected with the amplified immunoglobulin sequences, derived from in vivo selected lymphocytes, can then undergo further analysis and selection in vitro, for example, by panning the transfected cells to isolate cells expressing antibodies to GFAP. The amplified immunoglobulin sequences further can be manipulated in vitro, such as by in vitro affinity maturation method. See, for example, PCT Publication No. WO 97/29131 and PCT Publication No. WO 00/56772.

### (3) Anti-GFAP Monoclonal Antibodies Using Transgenic Animals

In another embodiment of the disclosure, antibodies are produced by immunizing a non-human animal comprising some, or all, of the human immunoglobulin locus with a GFAP antigen. In an embodiment, the non-human animal is a XENOMOUSE® transgenic mouse, an engineered mouse strain that comprises large fragments of the human immunoglobulin loci and is deficient in mouse antibody production. See, e.g., Green et al., Nature Genetics, 7:13-21 (1994) and U.S. Pat. Nos. 5,916,771; 5,939,598; 5,985,615; 5,998,209; 6,075,181; 6,091,001; 6,114,598; and 6,130,364. See also PCT Publication Nos. WO 91/10741; WO 94/02602; WO 96/34096; WO 96/33735; WO 98/16654; WO 98/24893; WO 98/50433; WO 99/45031; WO 99/53049; WO 00/09560; and WO 00/37504. The XENOMOUSE® transgenic mouse produces an adult-like human repertoire of fully human antibodies, and generates antigen-specific human monoclonal antibodies. The XENOMOUSE® transgenic mouse contains approximately 80% of the human antibody repertoire through introduction of megabase sized, germline configuration YAC fragments of the human heavy chain loci and x light chain loci. See Mendez et al., Nature Genetics, 15:146-156 (1997), Green and Jakobovits, J. Exp. Med., 188:483-495 (1998), the disclosures of which are hereby incorporated by reference.

### (4) Anti-GFAP Monoclonal Antibodies Using Recombinant Antibody Libraries

In vitro methods also can be used to make the antibodies of the disclosure, wherein an antibody library is screened to identify an antibody having the desired GFAP-binding specificity. Methods for such screening of recombinant antibody libraries are well known in the art and include methods described in, for example, U.S. Pat. No. 5,223,409 (Ladner et al.); PCT Publication No. WO 92/18619 (Kang et al.); PCT Publication No. WO 91/17271 (Dower et al.); PCT Publication No. WO 92/20791 (Winter et al.); PCT Publication No. WO 92/15679 (Markland et al.); PCT Publication No. WO 93/01288 (Breitling et al.); PCT Publication No. WO 92/01047 (McCafferty et al.); PCT Publication No. WO 92/09690 (Garrard et al.); Fuchs et al., Bio/Technology, 9:1369-1372 (1991); Hay et al., Hum. Antibod. Hybridomas, 3:81-85 (1992); Huse et al., Science, 246:1275-1281 (1989); McCafferty et al., Nature, 348:552-554 (1990); Griffiths et al., EMBO J., 12:725-734 (1993); Hawkins et al., J. Mol. Biol., 226:889-896 (1992); Clackson et al., Nature, 352:624-628 (1991); Gram et al., Proc. Natl. Acad. Sci. USA, 89:3576-3580 (1992); Garrard et al., Bio/Technology, 9:1373-1377 (1991); Hoogenboom et al., Nucl. Acids Res., 19:4133-4137 (1991); Barbas et al., Proc. Natl. Acad. Sci. USA, 88:7978-7982 (1991); U.S. Patent Application Publication No. 2003/0186374; and PCT Publication No. WO 97/29131, the contents of each of which are incorporated herein by reference.

The recombinant antibody library may be from a subject immunized with GFAP, or a portion of GFAP. Alternatively, the recombinant antibody library may be from a naive subject, i.e., one who has not been immunized with GFAP, such as a human antibody library from a human subject who has not been immunized with human GFAP. Antibodies of the disclosure are selected by screening the recombinant antibody library with the peptide comprising human GFAP to thereby select those antibodies that recognize GFAP. Methods for conducting such screening and selection are well known in the art, such as described in the references in the preceding paragraph. To select antibodies of the disclosure having particular binding affinities for GFAP, such as those that dissociate from human GFAP with a particular K_{off} rate constant, the art-known method of surface plasmon resonance can be used to select antibodies having the desired K_{off} rate constant. To select antibodies of the disclosure having a particular neutralizing activity for hGFAP, such as those with a particular IC₅₀, standard methods known in the art for assessing the inhibition of GFAP activity may be used.

In one embodiment, the disclosure pertains to an isolated antibody, or an antigen-binding portion thereof, that binds human GFAP. Preferably, the antibody is a neutralizing antibody. In various embodiments, the antibody is a recombinant antibody or a monoclonal antibody.

For example, antibodies can also be generated using various phage display methods known in the art. In phage display methods, functional antibody domains are displayed on the surface of phage particles which carry the polynucleotide sequences encoding them. Such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv, or disulfide stabilized Fv antibody domains recombinantly fused to either the phage gene III or gene VIII protein. Examples of phage display methods that can be used to make the antibodies include those disclosed in Brinkmann et al., J. Immunol. Methods, 182:41-50 (1995); Ames et al., J. Immunol. Methods, 184:177-186 (1995); Kettleborough et al., Eur. J. Immunol., 24:952-958 (1994); Persic et al., Gene, 187:9-18 (1997); Burton et al., Advances in Immunology, 57:191-280 (1994); PCT Publication No. WO 92/01047; PCT Publication Nos. WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

As described in the above references, after phage selection, the antibody coding regions from the phage can be isolated and used to generate whole antibodies including human antibodies or any other desired antigen binding fragment, and expressed in any desired host, including mammalian cells, insect cells, plant cells, yeast, and bacteria, e.g., as described in detail below. For example, techniques to recombinantly produce Fab, Fab′, and F(ab′)₂ fragments can also be employed using methods known in the art such as those disclosed in PCT publication No. WO 92/22324; Mullinax et al., BioTechniques, 12 (6): 864-869 (1992); Sawai et al., Am. J. Reprod. Immunol., 34:26-34 (1995); and Better et al., Science, 240:1041-1043 (1988). Examples of techniques which can be used to produce single-chain Fvs and antibodies include those described in U.S. Pat. Nos. 4,946,778 and 5,258,498; Huston et al., Methods in Enzymology, 203:46-88 (1991); Shu et al., Proc. Natl. Acad. Sci. USA, 90:7995-7999 (1993); and Skerra et al., Science, 240:1038-1041 (1988).

Alternative to screening of recombinant antibody libraries by phage display, other methodologies known in the art for screening large combinatorial libraries can be applied to the identification of antibodies of the disclosure. One type of alternative expression system is one in which the recombinant antibody library is expressed as RNA-protein fusions, as described in PCT Publication No. WO 98/31700 (Szostak and Roberts), and in Roberts and Szostak, Proc. Natl. Acad. Sci. USA, 94:12297-12302 (1997). In this system, a covalent fusion is created between an mRNA and the peptide or protein that it encodes by in vitro translation of synthetic mRNAs that carry puromycin, a peptidyl acceptor antibiotic, at their 3′ end. Thus, a specific mRNA can be enriched from a complex mixture of mRNAs (e.g., a combinatorial library) based on the properties of the encoded peptide or protein, e.g., antibody, or portion thereof, such as binding of the antibody, or portion thereof, to the dual specificity antigen. Nucleic acid sequences encoding antibodies, or portions thereof, recovered from screening of such libraries can be expressed by recombinant means as described above (e.g., in mammalian host cells) and, moreover, can be subjected to further affinity maturation by either additional rounds of screening of mRNA-peptide fusions in which mutations have been introduced into the originally selected sequence(s), or by other methods for affinity maturation in vitro of recombinant antibodies, as described above. A preferred example of this methodology is PROfusion display technology.

In another approach, the antibodies can also be generated using yeast display methods known in the art. In yeast display methods, genetic methods are used to tether antibody domains to the yeast cell wall and display them on the surface of yeast. Such yeast can be utilized to display antigen-binding domains expressed from a repertoire or combinatorial antibody library (e.g., human or murine). Examples of yeast display methods that can be used to make the antibodies include those disclosed in U.S. Pat. No. 6,699,658 (Wittrup et al.) incorporated herein by reference.

d. Production of Recombinant GFAP Antibodies

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the disclosure in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Exemplary mammalian host cells for expressing the recombinant antibodies of the disclosure include Chinese Hamster Ovary (CHO cells) (including dhfr-CHO cells, described in Urlaub and Chasin, Proc. Natl. Acad. Sci. USA, 77:4216-4220 (1980), used with a DHFR selectable marker, e.g., as described in Kaufman and Sharp, J. Mol. Biol., 159:601-621 (1982), NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure may be performed. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure (i.e., binds human GFAP) and the other heavy and light chain are specific for an antigen other than human GFAP by crosslinking an antibody of the disclosure to a second antibody by standard chemical crosslinking methods.

In a preferred system for recombinant expression of an antibody, or antigen-binding portion thereof, of the disclosure, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells, and recover the antibody from the culture medium. Still further, the disclosure provides a method of synthesizing a recombinant antibody of the disclosure by culturing a host cell of the disclosure in a suitable culture medium until a recombinant antibody of the disclosure is synthesized. The method can further comprise isolating the recombinant antibody from the culture medium.

### (1) Humanized Antibody

The humanized antibody may be an antibody or a variant, derivative, analog or portion thereof which immunospecifically binds to an antigen of interest and which comprises a framework (FR) region having substantially the amino acid sequence of a human antibody and a complementary determining region (CDR) having substantially the amino acid sequence of a non-human antibody. The humanized antibody may be from a non-human species antibody that binds the desired antigen having one or more complementarity determining regions (CDRs) from the non-human species and framework regions from a human immunoglobulin molecule.

As used herein, the term “substantially” in the context of a CDR refers to a CDR having an amino acid sequence at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. According to one embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or of a heavy chain.

The humanized antibody can be selected from any class of immunoglobulins, including IgM, IgG, IgD, IgA and IgE, and any isotype, including without limitation IgG 1, IgG2, IgG3, and IgG4. The humanized antibody may comprise sequences from more than one class or isotype, and particular constant domains may be selected to optimize desired effector functions using techniques well-known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In one embodiment, such mutations, however, will not be extensive. Usually, at least 90%, at least 95%, at least 98%, or at least 99% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

The humanized antibody may be designed to minimize unwanted immunological response toward rodent anti-human antibodies, which limits the duration and effectiveness of therapeutic applications of those moieties in human recipients. The humanized antibody may have one or more amino acid residues introduced into it from a source that is non-human. These non-human residues are often referred to as “import” residues, which are typically taken from a variable domain. Humanization may be performed by substituting hypervariable region sequences for the corresponding sequences of a human antibody. Accordingly, such “humanized” antibodies are chimeric antibodies wherein substantially less than an intact human variable domain has been substituted by the corresponding sequence from a non-human species. For example, see U.S. Pat. No. 4,816,567, the contents of which are herein incorporated by reference. The humanized antibody may be a human antibody in which some hypervariable region residues, and possibly some FR residues are substituted by residues from analogous sites in rodent antibodies. Humanization or engineering of antibodies of the present disclosure can be performed using any known method, such as but not limited to those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

The humanized antibody may retain high affinity for GFAP and other favorable biological properties. The humanized antibody may be prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristics, such as increased affinity for GFAP, is achieved. In general, the hypervariable region residues may be directly and most substantially involved in influencing antigen binding.

As an alternative to humanization, human antibodies (also referred to herein as “fully human antibodies”) can be generated. For example, it is possible to isolate human antibodies from libraries via PROfusion and/or yeast related technologies. It is also possible to produce transgenic animals (e.g. mice that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. The humanized or fully human antibodies may be prepared according to the methods described in U.S. Pat. Nos. 5,770,429; 5,833,985; 5,837,243; 5,922,845; 6,017,517; 6,096,311; 6,111,166; 6,270,765; 6,303,755; 6,365,116; 6,410,690; 6,682,928; and 6,984,720, the contents each of which are herein incorporated by reference.

e. Anti-GFAP Antibodies

Anti-GFAP antibodies may be generated using the techniques described above as well as using routine techniques known in the art. In some embodiments, the anti-GFAP antibody may be an unconjugated GFAP antibody, such as GFAP antibodies available from Dako (Catalog Number: M0761), ThermoFisher Scientific (Catalog Numbers: MA5-12023, A-21282, 13-0300. MA1-19170. MA1-19395, MA5-15086, MA5-16367, MA1-35377, MA1-06701, or MA1-20035), AbCam (Catalog Numbers: ab10062, ab4648, ab68428, ab33922, ab207165, ab 190288, ab115898, or ab21837), EMD Millipore (Catalog Numbers: FCMAB257P, MAB360, MAB3402, 04-1031, 04-1062, MAB5628), Santa Cruz (Catalog Numbers: sc-166481, sc-166458, sc-58766, sc-56395, sc-51908, sc-135921, sc-71143, sc-65343, or sc-33673), Sigma-Aldrich (Catalog Numbers: G3893 or G6171) or Sino Biological Inc. (Catalog Number: 100140-R012-50). The anti-GFAP antibody may be conjugated to a fluorophore, such as conjugated GFAP antibodies available from ThermoFisher Scientific (Catalog Numbers: A-21295 or A-21294). EMD Millipore (Catalog Numbers: MAB3402X, MAB3402B, MAB3402B, or MAB3402C3) or AbCam (Catalog Numbers: ab49874 or ab194325).

Alternatively, the antibodies described in WO 2018/067474, WO2018/081649, U.S. Pat. No. 11,078,298, U.S. Patent Publication No. 2019/0502127, and/or Bazarian et al., “Accuracy of a rapid GFAP/UCH-L1 test for the prediction of intracranial injuries on head CT after mild traumatic brain injury”, Acad. Emerg. Med., (Aug. 6, 2021), the contents of which are herein incorporated by reference, can also be used.

### 14. Variations on Methods

The disclosed methods of determining the presence or amount of analyte of interest (UCH-L1 and/or GFAP) present in a sample may be as described herein. The methods may also be adapted in view of other methods for analyzing analytes. Examples of well-known variations include, but are not limited to, immunoassay, such as sandwich immunoassay (e.g., monoclonal-monoclonal sandwich immunoassays, monoclonal-polyclonal sandwich immunoassays, including enzyme detection (enzyme immunoassay (EIA) or enzyme-linked immunosorbent assay (ELISA), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, etc.

a. Immunoassay

The analyte of interest, and/or peptides of fragments thereof (e.g., UCH-L1 and/or GFAP, and/or peptides or fragments thereof, i.e., UCH-L1 and/or GFAP fragments), may be analyzed using UCH-L1 and/or GFAP antibodies in an immunoassay. The presence or amount of analyte (e.g., UCH-L1 and/or GFAP) can be determined using antibodies and detecting specific binding to the analyte (e.g., UCH-L1 and/or GFAP). For example, the antibody, or antibody fragment thereof, may specifically bind to the analyte (e.g., UCH-L1 and/or GFAP). If desired, one or more of the antibodies can be used in combination with one or more commercially available monoclonal/polyclonal antibodies. Such antibodies are available from companies such as R&D Systems, Inc. (Minneapolis, MN) and Enzo Life Sciences International, Inc. (Plymouth Meeting, PA).

The presence or amount of analyte (e.g., UCH-L1 and/or GFAP) present in a body sample may be readily determined using an immunoassay, such as sandwich immunoassay (e.g., monoclonal-monoclonal sandwich immunoassays, monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, MN)). An example of a point-of-care device that can be used is i-STAT (Abbott, Laboratories, Abbott Park, IL). Other methods that can be used include a chemiluminescent microparticle immunoassay, in particular those employing the ARCHITECT or Alinity automated series of analyzers (Abbott Laboratories, Abbott Park, IL), as an example. Other methods include, for example, mass spectrometry, and immunohistochemistry (e.g., with sections from tissue biopsies), using anti-analyte (e.g., anti-UCH-L1 and/or anti-GFAP) antibodies (monoclonal, polyclonal, chimeric, humanized, human, etc.) or antibody fragments thereof against analyte (e.g., UCH-L1 and/or GFAP). Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antibody to the analyte (e.g., UCH-L1 and/or GFAP) can be detected via direct labels, such as fluorescent or luminescent tags, metals and radionuclides attached to the antibody or via indirect labels, such as alkaline phosphatase or horseradish peroxidase.

The use of immobilized antibodies or antibody fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

A homogeneous format may be used. For example, after the test sample is obtained from a subject, a mixture is prepared. The mixture contains the test sample being assessed for analyte (e.g., UCH-L1 and/or GFAP), a first specific binding partner, and a second specific binding partner. The order in which the test sample, the first specific binding partner, and the second specific binding partner are added to form the mixture is not critical. The test sample is simultaneously contacted with the first specific binding partner and the second specific binding partner. In some embodiments, the first specific binding partner and any UCH-L1 and/or GFAP contained in the test sample may form a first specific binding partner-analyte (e.g., UCH-L1 and/or GFAP)-antigen complex and the second specific binding partner may form a first specific binding partner-analyte of interest (e.g., UCH-L1 and/or GFAP)-second specific binding partner complex. In some embodiments, the second specific binding partner and any UCH-L1 and/or GFAP contained in the test sample may form a second specific binding partner-analyte (e.g., UCH-L1)-antigen complex and the first specific binding partner may form a first specific binding partner-analyte of interest (e.g., UCH-L1 and/or GFAP)-second specific binding partner complex. The first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

A heterogeneous format may be used. For example, after the test sample is obtained from a subject, a first mixture is prepared. The mixture contains the test sample being assessed for analyte (e.g., UCH-L1 and/or GFAP) and a first specific binding partner, wherein the first specific binding partner and any UCH-L1 and/or GFAP contained in the test sample form a first specific binding partner-analyte (e.g., UCH-L1 and/or GFAP)-antigen complex. The first specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). The order in which the test sample and the first specific binding partner are added to form the mixture is not critical.

The first specific binding partner may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, and a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fc. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The solid support on which the first specific binding member is immobilized may be stored in dry form or in a liquid. The magnetic beads may be subjected to a magnetic field prior to or after contacting with the sample with a magnetic bead on which the first specific binding member is immobilized.

After the mixture containing the first specific binding partner-analyte (e.g., UCH-L1 or GFAP) antigen complex is formed, any unbound analyte (e.g., UCH-L1 and/or GFAP) is removed from the complex using any technique known in the art. For example, the unbound analyte (e.g., UCH-L1 and/or GFAP) can be removed by washing. Desirably, however, the first specific binding partner is present in excess of any analyte (e.g., UCH-L1 and/or GFAP) present in the test sample, such that all analyte (e.g., UCH-L1 and/or GFAP) that is present in the test sample is bound by the first specific binding partner.

After any unbound analyte (e.g., UCH-L1 and/or GFAP) is removed, a second specific binding partner is added to the mixture to form a first specific binding partner-analyte of interest (e.g., UCH-L1 and/or GFAP)-second specific binding partner complex. The second specific binding partner may be an anti-analyte antibody (e.g., anti-UCH-L1 antibody that binds to an epitope having an amino acid sequence comprising at least three contiguous (3) amino acids of SEQ ID NO: 1 or anti-GFAP antibody that binds to an epitope having an amino acid sequence comprising at least three contiguous (3) amino acids of SEQ ID NO: 2). Moreover, the second specific binding partner is labeled with or contains a detectable label as described above.

The use of immobilized antibodies or antibody fragments thereof may be incorporated into the immunoassay. The antibodies may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. An assay strip can be prepared by coating the antibody or plurality of antibodies in an array on a solid support. This strip can then be dipped into the test sample and processed quickly through washes and detection steps to generate a measurable signal, such as a colored spot.

In some embodiments, it is possible that other antibodies can be selected which similarly may assist with maintaining the dynamic range and low end sensitivity of the immunoassays. For example, it may be useful to select at least one first antibody (such as a capture antibody or first specific binding partner) that binds to an epitope near the N-terminus of the 38 kDa BDP and at least one second antibody (such as a detection antibody or second specific binding partner) that binds to an epitope near the middle of the 38 kDa BDP, e.g., near the middle of the 38 kDa BDP, and that does not overlap with the first antibody. Other variations are possible and could be readily tested by one of ordinary skill, such as by confirming antibodies bind to different epitopes by examining binding to short peptides, and then screening antibody pairs using low calibrator concentration. Moreover, selecting antibodies of differing affinity for GFAP also can assist with maintaining or increasing the dynamic range of the assay. GFAP antibodies have been described in the literature and are commercially available.

### (1) Sandwich Immunoassay

A sandwich immunoassay measures the amount of antigen between two layers of antibodies (i.e., at least one capture antibody) and a detection antibody (i.e., at least one detection antibody). The capture antibody and the detection antibody bind to different epitopes on the antigen, e.g., analyte of interest such as UCH-L1 and/or GFAP. Desirably, binding of the capture antibody to an epitope does not interfere with binding of the detection antibody to an epitope. Either monoclonal or polyclonal antibodies may be used as the capture and detection antibodies in the sandwich immunoassay.

Generally, at least two antibodies are employed to separate and quantify analyte (e.g., UCH-L1 and/or GFAP) in a test sample. More specifically, the at least two antibodies bind to certain epitopes of analyte (e.g., UCH-L1 and/or GFAP) forming an immune complex which is referred to as a “sandwich”. One or more antibodies can be used to capture the analyte (e.g., UCH-L1 and/or GFAP) in the test sample (these antibodies are frequently referred to as a “capture” antibody or “capture” antibodies) and one or more antibodies is used to bind a detectable (namely, quantifiable) label to the sandwich (these antibodies are frequently referred to as the “detection” antibody or “detection” antibodies). In a sandwich assay, the binding of an antibody to its epitope desirably is not diminished by the binding of any other antibody in the assay to its respective epitope. Antibodies are selected so that the one or more first antibodies brought into contact with a test sample suspected of containing analyte (e.g., UCH-L1 and/or GFAP) do not bind to all or part of an epitope recognized by the second or subsequent antibodies, thereby interfering with the ability of the one or more second detection antibodies to bind to the analyte (e.g., UCH-L1 and/or GFAP).

The antibodies may be used as a first antibody in said immunoassay. The antibody immunospecifically binds to epitopes on analyte (e.g., UCH-L1 and/or GFAP). In addition to the antibodies of the present disclosure, said immunoassay may comprise a second antibody that immunospecifically binds to epitopes that are not recognized or bound by the first antibody.

A test sample suspected of containing analyte (e.g., UCH-L1 and/or GFAP) can be contacted with at least one first capture antibody (or antibodies) and at least one second detection antibodies either simultaneously or sequentially. In the sandwich assay format, a test sample suspected of containing analyte (e.g., UCH-L1 and/or GFAP) is first brought into contact with the at least one first capture antibody that specifically binds to a particular epitope under conditions which allow the formation of a first antibody-analyte (e.g., UCH-L1 and/or GFAP) antigen complex. If more than one capture antibody is used, a first multiple capture antibody-UCH-L1 and/or GFAP antigen complex is formed. In a sandwich assay, the antibodies, preferably, the at least one capture antibody, are used in molar excess amounts of the maximum amount of analyte (e.g., UCH-L1 and/or GFAP) expected in the test sample. For example, from about 5 μg/mL to about 1 mg/mL of antibody per ml of microparticle coating buffer may be used.

i. Anti-UCH-L1 Capture Antibody

Optionally, prior to contacting the test sample with the at least one first capture antibody, the at least one first capture antibody can be bound to a solid support which facilitates the separation the first antibody-analyte (e.g., UCH-L1 and/or GFAP) complex from the test sample. Any solid support known in the art can be used, including but not limited to, solid supports made out of polymeric materials in the forms of wells, tubes, or beads (such as a microparticle). The antibody (or antibodies) can be bound to the solid support by adsorption, by covalent bonding using a chemical coupling agent or by other means known in the art, provided that such binding does not interfere with the ability of the antibody to bind analyte (e.g., UCH-L1 and/or GFAP). Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl) carbodiimide.

After the test sample suspected of containing analyte (e.g., UCH-L1 and/or GFAP) is incubated in order to allow for the formation of a first capture antibody (or multiple antibody)-analyte (e.g., UCH-L1 and/or GFAP) complex. The incubation can be carried out at a pH of from about 4.5 to about 10.0, at a temperature of from about 2° C. to about 45° C., and for a period from at least about one (1) minute to about eighteen (18) hours, from about 2-6 minutes, from about 7-12 minutes, from about 5-15 minutes, or from about 3-4 minutes.

ii. Detection Antibody

After formation of the first/multiple capture antibody-analyte (e.g., UCH-L1 and/or GFAP) complex, the complex is then contacted with at least one second detection antibody (under conditions that allow for the formation of a first/multiple antibody-analyte (e.g., UCH-L1 and/or GFAP) antigen-second antibody complex). In some embodiments, the test sample is contacted with the detection antibody simultaneously with the capture antibody. If the first antibody-analyte (e.g., UCH-L1 and/or GFAP) complex is contacted with more than one detection antibody, then a first/multiple capture antibody-analyte (e.g., UCH-L1 and/or GFAP)-multiple antibody detection complex is formed. As with first antibody, when the at least second (and subsequent) antibody is brought into contact with the first antibody-analyte (e.g., UCH-L1 and/or GFAP) complex, a period of incubation under conditions similar to those described above is required for the formation of the first/multiple antibody-analyte (e.g., UCH-L1 and/or GFAP)-second/multiple antibody complex. Preferably, at least one second antibody contains a detectable label. The detectable label can be bound to the at least one second antibody prior to, simultaneously with or after the formation of the first/multiple antibody-analyte (e.g., UCH-L1 and/or GFAP)-second/multiple antibody complex. Any detectable label known in the art can be used.

Chemiluminescent assays can be performed in accordance with the methods described in Adamczyk et al., Anal. Chim. Acta 579 (1): 61-67 (2006). While any suitable assay format can be used, a microplate chemiluminometer (Mithras LB-940, Berthold Technologies U.S.A., LLC, Oak Ridge, TN) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the test sample and the specific binding partner(s) are added to form the mixture for chemiluminescent assay is not critical. If the first specific binding partner is detectably labeled with an acridinium compound, detectably labeled first specific binding partner-antigen (e.g., UCH-L1 and/or GFAP) complexes form. Alternatively, if a second specific binding partner is used and the second specific binding partner is detectably labeled with an acridinium compound, detectably labeled first specific binding partner-analyte (e.g., UCH-L1 and/or GFAP)-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of analyte (e.g., UCH-L1 and/or GFAP) is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample. Other labels other than chemiluminescent labels can be employed. For instance, enzymatic labels (including but not limited to alkaline phosphatase) can be employed.

The chemiluminescent signal, or other signal, that is generated can be detected using routine techniques known to those skilled in the art. Based on the intensity of the signal generated, the amount of analyte of interest (e.g., UCH-L1 and/or GFAP) in the sample can be quantified. Specifically, the amount of analyte (e.g., UCH-L1 and/or GFAP) in the sample is proportional to the intensity of the signal generated. The amount of analyte (e.g., UCH-L1 and/or GFAP) present can be quantified by comparing the amount of light generated to a standard curve for analyte (e.g., UCH-L1 and/or GFAP) or by comparison to a reference standard. The standard curve can be generated using serial dilutions or solutions of known concentrations of analyte (e.g., UCH-L1 and/or GFAP) by mass spectroscopy, gravimetric methods, and other techniques known in the art.

### (2) Forward Competitive Inhibition Assay

In a forward competitive format, an aliquot of labeled analyte of interest (e.g., analyte (e.g., UCH-L1 and/or GFAP) having a fluorescent label, a tag attached with a cleavable linker, etc.) of a known concentration is used to compete with analyte of interest (e.g., UCH-L1 and/or GFAP) in a test sample for binding to analyte of interest antibody (e.g., UCH-L1 and/or GFAP antibody).

In a forward competition assay, an immobilized specific binding partner (such as an antibody) can either be sequentially or simultaneously contacted with the test sample and a labeled analyte of interest, analyte of interest fragment or analyte of interest variant thereof. The analyte of interest peptide, analyte of interest fragment or analyte of interest variant can be labeled with any detectable label, including a detectable label comprised of tag attached with a cleavable linker. In this assay, the antibody can be immobilized on to a solid support. Alternatively, the antibody can be coupled to an antibody, such as an antispecies antibody, that has been immobilized on a solid support, such as a microparticle or planar substrate.

The labeled analyte of interest, the test sample and the antibody are incubated under conditions similar to those described above in connection with the sandwich assay format. Two different species of antibody-analyte of interest complexes may then be generated. Specifically, one of the antibody-analyte of interest complexes generated contains a detectable label (e.g., a fluorescent label, etc.) while the other antibody-analyte of interest complex does not contain a detectable label. The antibody-analyte of interest complex can be, but does not have to be, separated from the remainder of the test sample prior to quantification of the detectable label. Regardless of whether the antibody-analyte of interest complex is separated from the remainder of the test sample, the amount of detectable label in the antibody-analyte of interest complex is then quantified. The concentration of analyte of interest (such as membrane-associated analyte of interest, soluble analyte of interest, fragments of soluble analyte of interest, variants of analyte of interest (membrane-associated or soluble analyte of interest) or any combinations thereof) in the test sample can then be determined, e.g., as described above.

### (3) Reverse Competitive Inhibition Assay

In a reverse competition assay, an immobilized analyte of interest (e.g., UCH-L1 and/or GFAP) can either be sequentially or simultaneously contacted with a test sample and at least one labeled antibody.

The analyte of interest can be bound to a solid support, such as the solid supports discussed above in connection with the sandwich assay format.

The immobilized analyte of interest, test sample and at least one labeled antibody are incubated under conditions similar to those described above in connection with the sandwich assay format. Two different species analyte of interest-antibody complexes are then generated. Specifically, one of the analyte of interest-antibody complexes generated is immobilized and contains a detectable label (e.g., a fluorescent label, etc.) while the other analyte of interest-antibody complex is not immobilized and contains a detectable label. The non-immobilized analyte of interest-antibody complex and the remainder of the test sample are removed from the presence of the immobilized analyte of interest-antibody complex through techniques known in the art, such as washing. Once the non-immobilized analyte of interest antibody complex is removed, the amount of detectable label in the immobilized analyte of interest-antibody complex is then quantified following cleavage of the tag. The concentration of analyte of interest in the test sample can then be determined by comparing the quantity of detectable label as described above.

### (4) One-Step Immunoassay or “Capture on the Fly” Assay

In a capture on the fly immunoassay, a solid substrate is pre-coated with an immobilization agent. The capture reagent, the analyte (e.g., UCH-L1 and/or GFAP) and the detection reagent are added to the solid substrate together, followed by a wash step prior to detection. The capture reagent can bind the analyte (e.g., UCH-L1 and/or GFAP) and comprises a ligand for an immobilization agent. The capture reagent and the detection reagents may be antibodies or any other moiety capable of capture or detection as described herein or known in the art. The ligand may comprise a peptide tag and an immobilization agent may comprise an anti-peptide tag antibody. Alternately, the ligand and the immobilization agent may be any pair of agents capable of binding together so as to be employed for a capture on the fly assay (e.g., specific binding pair, and others such as are known in the art). More than one analyte may be measured. In some embodiments, the solid substrate may be coated with an antigen and the analyte to be analyzed is an antibody.

In certain other embodiments, in a one-step immunoassay or “capture on the fly”, a solid support (such as a microparticle) pre-coated with an immobilization agent (such as biotin, streptavidin, etc.) and at least a first specific binding member and a second specific binding member (which function as capture and detection reagents, respectively) are used. The first specific binding member comprises a ligand for the immobilization agent (for example, if the immobilization agent on the solid support is streptavidin, the ligand on the first specific binding member may be biotin) and also binds to the analyte of interest (e.g., UCH-L1 and/or GFAP). The second specific binding member comprises a detectable label and binds to an analyte of interest (e.g., UCH-L1 and/or GFAP). The solid support and the first and second specific binding members may be added to a test sample (either sequentially or simultaneously). The ligand on the first specific binding member binds to the immobilization agent on the solid support to form a solid support/first specific binding member complex. Any analyte of interest present in the sample binds to the solid support/first specific binding member complex to form a solid support/first specific binding member/analyte complex. The second specific binding member binds to the solid support/first specific binding member/analyte complex and the detectable label is detected. An optional wash step may be employed before the detection. In certain embodiments, in a one-step assay more than one analyte may be measured. In certain other embodiments, more than two specific binding members can be employed. In certain other embodiments, multiple detectable labels can be added. In certain other embodiments, multiple analytes of interest can be detected, or their amounts, levels or concentrations, measured, determined or assessed.

The use of a capture on the fly assay can be done in a variety of formats as described herein, and known in the art. For example the format can be a sandwich assay such as described above, but alternately can be a competition assay, can employ a single specific binding member, or use other variations such as are known.

### 15. Other Factors

The methods of diagnosing, prognosticating, and/or assessing, as described above, can further include using other factors for the diagnosis, prognostication, and assessment. In some embodiments, traumatic brain injury may be diagnosed using the Glasgow Coma Scale or the Extended Glasgow Outcome Scale (GOSE). Other tests, scales or indices can also be used either alone or in combination with the Glasgow Coma Scale. An example is the Ranchos Los Amigos Scale. The Ranchos Los Amigos Scale measures the levels of awareness, cognition, behavior and interaction with the environment. The Ranchos Los Amigos Scale includes: Level I: No Response; Level II: Generalized Response; Level III: Localized Response; Level IV: Confused-agitated; Level V: Confused-inappropriate; Level VI: Confused-appropriate; Level VII: Automatic-appropriate; and Level VIII: Purposeful-appropriate. Another example is the Rivermead Post-Concussion Symptoms Questionnaire, a self-report scale to measure the severity of post-concussive symptoms following TBI. Patients are asked to rate how severe each of 16 symptoms (e.g., headache, dizziness, nausea, vomiting) has been over the past 24 hours. In each case, the symptom is compared with how severe it was before the injury occurred (premorbid). These symptoms are reported by severity on a scale from 0 to 4: not experienced, no more of a problem, mild problem, moderate problem, and severe problem.

### 16. Samples

In some embodiments, the sample is obtained after the subject, such as a human subject, sustained an injury to the head caused by physical shaking, blunt impact by an external mechanical or other force that results in a closed or open head trauma, one or more falls, explosions or blasts or other types of blunt force trauma. In some embodiments, the sample is obtained after the subject, such as a human subject, has ingested or been exposed to a fire, chemical, toxin or combination of a fire, chemical and toxin. Examples of such chemicals and/or toxins include, molds, asbestos, pesticides and insecticides, organic solvents, paints, glues, gases (such as carbon monoxide, hydrogen sulfide, and cyanide), organic metals (such as methyl mercury, tetraethyl lead and organic tin) and/or one or more drugs of abuse. In some embodiments, the sample is obtained from a subject, such as a human subject, that suffers from an autoimmune disease, a metabolic disorder, a brain tumor, hypoxia, a viral infection (e.g., SARS-CoV-2), a fungal infection, a bacterial infection, meningitis, hydrocephalus, or any combinations thereof.

In yet another embodiment, the methods described herein use samples that also can be used to determine whether or not a subject has or is at risk of developing a TBI (such as a mild TBI, moderate TBI, severe TBI, or moderate to severe TBI) by determining the levels of UCH-L1 and/or GFAP in a subject using the anti-UCH-L1 and/or anti-GFAP antibodies described below, or antibody fragments thereof. Thus, in particular embodiments, the disclosure also provides a method for determining whether a subject having, or at risk for, traumatic brain injuries, discussed herein and known in the art, is a candidate for therapy or treatment. Generally, the subject is at least one who: (i) has experienced an injury to the head; (ii) ingested and/or been exposed to one or more chemicals and/or toxins; (iii) suffers from an autoimmune disease, a metabolic disorder, a brain tumor, hypoxia, a viral infection (e.g., SARS-CoV-2), a fungal infection, a bacterial infection, meningitis, hydrocephalus, or any combinations thereof; or (iv) any combinations of (i)-(iii); or, who has actually been diagnosed as having, or being at risk for TBI (such as, for example, subjects suffering from an autoimmune disease, a metabolic disorder, a brain tumor, hypoxia, a viral infection (e.g., SARS-CoV-2), a fungal infection, a bacterial infection, meningitis, hydrocephalus, or any combinations thereof), and/or who demonstrates an unfavorable (i.e., clinically undesirable) concentration or amount of UCH-L1 and/or GFAP or UCH-L1 and/or GFAP fragment, as described herein.

b. Test or Biological Sample

As used herein, “sample”, “test sample”, “biological sample” refer to fluid sample containing or suspected of containing GFAP and/or UCH-L1. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing GFAP and/or UCH-L1 may be assayed directly. In a particular example, the source containing GFAP and/or UCH-L1 is a human (e.g., pediatric or adult human) substance or substance from another species. As used herein, the term “pediatric” or “pediatric subject” refers to a subject less than 18 years of age (i.e., not 18 years of age or older). For example, a pediatric subject may be less than about 18 years old, or about 17 years old, about 16 years old, about 15 years old, about 14 years old, about 13 years old, about 12 years old, about 11 years old, about 10 years old, about 9 years old, about 8 years old, about 7 years old, about 6 years old, about 5 years old, about 4 years old, about 3 years old, about 2 years old, about 1 year old, or less than about 1 year old. In some embodiments, the pediatric subject may be less than about 1 year old to about less than 18 years old. In some embodiment, the pediatric subject may be less than about 1 year old to about 17 years old. For example, a pediatric subject may be anywhere from about one day, about two days, about three days, about four days, about five days, about six days, about one week, about two weeks, about three weeks, about one month, about two months, about three months, about four months, about five months, about six months, about seven months, about eight months, about nine months, about ten months, or about eleven months, in total, less than: about 18 years old, or about 17 years old, or about 16 years old, or about 15 years old, or about 14 years old, or about 13 years old, or about 12 years old, or about 11 years old, or about 10 years old, or about 9 years old, or about 8 years old, or about 7 years old, or about 6 years old, or about 5 years old, or about 4 years old, or about 3 years old, or about 2 years old, or about 1 year old, or less than about 1 year old. An “adult” or an “adult subject” refers to a subject 18 years of age or older.

The substance optionally is a bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, oropharyngeal specimen, nasopharyngeal specimen, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. In some embodiments, the sample is a whole blood sample, a serum sample, a cerebrospinal fluid sample, a mixed sample of venous and capillary blood, a mixed sample of capillary blood and interstitial fluid, a tissue sample, a bodily fluid, or a plasma sample.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL.

In some cases, the fluid sample may be diluted prior to use in an assay. For example, in embodiments where the source containing GFAP and/or UCH-L1 is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

In some cases, the sample may undergo pre-analytical processing. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art. In some cases, the fluid sample may be concentrated prior to use in an assay. For example, in embodiments where the source containing GFAP and/or UCH-L1 is a body fluid (e.g., blood, serum) from a subject (e.g., human or other species), the fluid may be concentrated by precipitation, evaporation, filtration, centrifugation, or a combination thereof. A fluid sample may be concentrated about 1-fold, about 2-fold, about 3-fold, about 4-fold, about 5-fold, about 6-fold, about 10-fold, about 100-fold, or greater, prior to use.

c. Controls

It may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e., fluorescent signal intensity, if a fluorescent label is used. Using samples taken from multiple donors, standard curves can be provided for reference levels of the UCH-L1 and/or GFAP in normal healthy tissue, as well as for “at-risk” levels of the UCH-L1 and/or GFAP in tissue taken from donors, who may have one or more of the characteristics set forth above.

Thus, in view of the above, a method for determining the presence, amount, or concentration of UCH-L1 and/or GFAP in a test sample is provided. The method comprises assaying the test sample for UCH-L1 and/or GFAP by an immunoassay, for example, employing at least one capture antibody that binds to an epitope on UCH-L1 and/or GFAP and at least one detection antibody that binds to an epitope on UCH-L1 and/or GFAP which is different from the epitope for the capture antibody and optionally includes a detectable label, and comprising comparing a signal generated by the detectable label as a direct or indirect indication of the presence, amount or concentration of UCH-L1 and/or GFAP in the test sample to a signal generated as a direct or indirect indication of the presence, amount or concentration of UCH-L1 and/or GFAP in a calibrator. The calibrator is optionally, and is preferably, part of a series of calibrators in which each of the calibrators differs from the other calibrators in the series by the concentration of UCH-L1 and/or GFAP.

### 17. Kit

Provided herein is a kit, which may be used to determine the amount, concentration, or level at least one biomarker in a biological sample. In some embodiments, the kit comprises at least one nuclease. In some embodiments, the at least one nuclease is an endonuclease. In still other aspects, the at least one nuclease is a Benzonase® nuclease. In some embodiments, the at least one nuclease is present in the kit in the form of a solution. In still other embodiments, the solution contains at least 1 U/mL of the nuclease. In still other embodiments, the solution contains about 1 U/mL to about 1.5 U/mL of the nuclease. In yet other embodiments, the kit can further comprise one or more salts, such as magnesium chloride. The one or more salts can be contained in the solution containing the at least one nuclease or can be provided as a separate solution.

In yet other embodiments, in addition to the nuclease, the kit can further comprise at least one capture antibody and at least one detection antibody. In yet further embodiments, kit can comprise instructions for assaying the test sample for at least one biomarker by immunoassay, e.g., chemiluminescent microparticle immunoassay, a clinical chemistry assay, or any other assay known in the art. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. Alternatively or additionally, the kit can comprise a calibrator or control for the at least one biomarker and/or at least one container (e.g., tube, microtiter plates or strips, which can be already coated with the relevant biomarker for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

In some embodiments, the at least one biomarker is an ABI biomarker. In yet other embodiments, the biomarker is a TBI biomarker. In some embodiments, the kit is useful for assay a test sample for UCH-L1 and/or GFAP or UCH-L1 and/or GFAP fragment. The kit comprises at least one component for assaying the test sample for the TBI biomarker (e.g. UCH-L1 and/or GFAP) instructions for assaying the test sample. For example, the kit can comprise instructions for assaying the test sample for UCH-L1 and/or GFAP by immunoassay, e.g., chemiluminescent microparticle immunoassay. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like.

The at least one component may include at least one composition comprising one or more isolated antibodies or antibody fragments thereof that specifically bind to UCH-L1 and/or GFAP. The antibody may be a UCH-L1 and/or GFAP capture antibody and/or a UCH-L1 and/or GFAP detection antibody. In some embodiments, the kit additionally comprises a nuclease.

Alternatively or additionally, the kit can comprise a calibrator or control, e.g., purified, and optionally lyophilized, UCH-L1 and/or GFAP and/or at least one container (e.g., tube, microtiter plates or strips, which can be already coated with an anti-UCH-L1 and/or GFAP monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying UCH-L1 and/or GFAP. The reference standards may be employed to establish standard curves for interpolation and/or extrapolation of UCH-L1 and/or GFAP concentrations. The reference standards may include a high UCH-L1 and/or GFAP concentration level, for example, about 100000 μg/mL, about 125000 μg/mL, about 150000 μg/mL, about 175000 μg/mL, about 200000 μg/mL, about 225000 μg/mL, about 250000 μg/mL, about 275000 μg/mL, or about 300000 μg/mL; a medium UCH-L1 and/or GFAP concentration level, for example, about 25000 μg/mL, about 40000 μg/mL, about 45000 μg/mL, about 50000 μg/mL, about 55000 μg/mL, about 60000 μg/mL, about 75000 μg/mL or about 100000 μg/mL; and/or a low UCH-L1 and/or GFAP concentration level, for example, about 1 μg/mL, about 5 μg/mL, about 10 μg/mL, about 12.5 pg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, or about 100 μg/mL.

Any antibodies, which are provided in the kit, such as recombinant antibodies specific for UCH-L1 and/or GFAP, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., UCH-L1 and/or GFAP) or reagents for detecting the analyte (e.g., UCH-L1 and/or GFAP). The antibodies, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pre-treatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

a. Adaptation of Kit and Method

The kit (or components thereof), as well as the method for assessing or determining the concentration of UCH-L1 and/or GFAP in a test sample by an immunoassay as described herein, can be adapted for use in a variety of automated and semi-automated systems (including those wherein the solid phase comprises a microparticle), as described, e.g., U.S. Pat. No. 5,063,081, U.S. Patent Application Publication Nos. 2003/0170881, 2004/0018577, 2005/0054078, and 2006/0160164 and as commercially marketed e.g., by Abbott Laboratories (Abbott Park, IL) as Abbott Point of Care (i-STAT or i-STAT Alinity, Abbott Laboratories) as well as those described in U.S. Pat. Nos. 5,089,424 and 5,006,309, and as commercially marketed, e.g., by Abbott Laboratories (Abbott Park, IL) as ARCHITECT® or the series of Abbott Alinity devices.

Some of the differences between an automated or semi-automated system as compared to a non-automated system (e.g., ELISA) include the substrate to which the first specific binding partner (e.g., analyte antibody or capture antibody) is attached (which can affect sandwich formation and analyte reactivity), and the length and timing of the capture, detection, and/or any optional wash steps. Whereas a non-automated format such as an ELISA may require a relatively longer incubation time with sample and capture reagent (e.g., about 2 hours), an automated or semi-automated format (e.g., ARCHITECT®, Alinity, and any successor platform, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®, Alinity, and any successor platform) may have a relatively shorter incubation time (e.g., approximately 4 minutes for the ARCHITECT® and any successor platform).

Other platforms available from Abbott Laboratories include, but are not limited to, Alinity, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits, and kit components can be employed in other formats, for example, on electrochemical or other hand-held or point-of-care assay systems. As mentioned previously, the present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Patent App. Publication Nos. 2003/0170881, 2004/0018577, 2005/0054078, and 2006/0160164, which are incorporated in their entireties by reference for their teachings regarding same.

Regarding the adaptation of an assay to the i-STAT system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an i-STAT® cartridge with a fluidics format suitable for immunoassay. On a portion of the silicon chip, there is a specific binding partner for UCH-L1 and/or GFAP, such as one or more UCH-L1 and/or GFAP antibodies (one or more monoclonal/polyclonal antibody or a fragment thereof, a variant thereof, or a fragment of a variant thereof that can bind UCH-L1 and/or GFAP) or one or more anti-UCH-L1 and/or GFAP DVD-Igs (or a fragment thereof, a variant thereof, or a fragment of a variant thereof that can bind UCH-L1 and/or GFAP), either of which can be detectably labeled. Within the fluid pouch of the cartridge is an aqueous reagent that includes p-aminophenol phosphate.

In operation, a sample from a subject suspected of suffering from TBI is added to the holding chamber of the test cartridge, and the cartridge is inserted into the i-STAT reader. A pump element within the cartridge pushes the sample into a conduit containing the chip. The sample is brought into contact with the sensors allowing the enzyme conjugate to dissolve into the sample. The sample is oscillated across the sensors to promote formation of the sandwich of approximately 2-12 minutes. In the penultimate step of the assay, the sample is pushed into a waste chamber and wash fluid, containing a substrate for the alkaline phosphatase enzyme, is used to wash excess enzyme conjugate and sample off the sensor chip. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of UCH-L1 and/or GFAP in the sample by means of an embedded algorithm and factory-determined calibration curve.

The automated and semi-automated systems described herein for use in the methods of the present disclosure can utilize one or more computer programs, software or algorithms to provide the determination (readout) of whether to perform an CT scan (e.g., based on a positive result) or not to perform an CT scan (e.g., based on a negative result). For example, the computer program(s) or software (e.g., making use of an algorithm) can provide an interpretation (regardless of whether one, two or more samples are used) that: (1) when the level of GFAP and UCH-L1 is less than the reference level (or cutoff) that the result is negative meaning that no CT scan will be performed; or (2) when the level or GFAP and/or UCH-L1 is greater than or equal to the reference level (or cutoff) that the result is positive meaning that an CT scan will be performed. The computer program(s) or software can provide other appropriate interpretations, such as, whether the reference level is or is not correlated with a positive head CT, the presence of an intracranial lesion or with control subjects that have not suffered a traumatic brain injury, whether the subject suffering from the TBI should be monitored and/or treated with a TBI treatment, etc. Such computer programs or software are well known in the art.

The methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT conjugate diluent employed in certain kits (Abbott Laboratories, Abbott Park, IL) and containing 2-(N-morpholino) ethanesulfonic acid (MES), a salt, a protein blocker, an antimicrobial agent, and a detergent. An exemplary calibrator diluent is ARCHITECT human calibrator diluent employed in certain kits (Abbott Laboratories, Abbott Park, IL), which comprises a buffer containing MES, other salt, a protein blocker, and an antimicrobial agent. Additionally, as described in U.S. Patent Application No. 61/142,048 filed Dec. 31, 2008, improved signal generation may be obtained, e.g., in an i-STAT cartridge format, using a nucleic acid sequence linked to the signal antibody as a signal amplifier.

While certain embodiments herein are advantageous when employed to assess disease, such as traumatic brain injury, the assays and kits also optionally can be employed to assess UCH-L1 and/or GFAP in other diseases, disorders, and conditions as appropriate.

The method of assay also can be used to identify a compound that ameliorates diseases, such as traumatic brain injury. For example, a cell that expresses UCH-L1 and/or GFAP can be contacted with a candidate compound. The level of expression of UCH-L1 and/or GFAP in the cell contacted with the compound can be compared to that in a control cell using the method of assay described herein.

The present disclosure has multiple embodiments, illustrated by the following non-limiting examples.

### 18. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple embodiments, illustrated by the following non-limiting examples.

### Example 1

### i-STAT UCH-L1 Assay

The i-STAT UCH-L1 assay was used in a TBI patient population study. Monoclonal antibody pairs, such as Antibody A as a capture monoclonal antibody and Antibody B and C as a detection monoclonal antibody, were used. Antibody A is an exemplary anti-UCH-L1 antibody that was internally developed at Abbott Laboratories (Abbott Park, IL). Antibody B and C recognize different epitopes of UCH-L1 and enhance the detection of antigen in the sample that were developed by Banyan Biomarkers (Alachua, Florida). Other antibodies that were internally developed at Abbott Laboratories (Abbott Park, IL) also show or are expected to show similar enhancement of signal when used together as capture antibodies or detection antibodies, in various combinations. The UCH-L1 assay design was evaluated against key performance attributes. The cartridge configuration was Antibody Configuration: Antibody A (Capture Antibody)/Antibody B+C (Detection Antibody); Reagent conditions: 0.8% solids, 125 μg/mL Antibody Alkaline Phosphatase conjugate; and Sample Inlet Print: UCH-L1 standard. The assay time was 10-15 min (with 7-12 min sample capture time).

### Example 2

### i-STAT GFAP Assay

The i-STAT GFAP assay was used in a TBI patient population study. Monoclonal antibody pairs, such as Antibody A as a capture monoclonal antibody and Antibody B as a detection monoclonal antibody, were used. Antibody A and Antibody B are exemplary anti-GFAP antibodies that were internally developed at Abbott Laboratories (Abbott Park, IL). The GFAP assay design was evaluated against key performance attributes. The cartridge configuration was Antibody Configuration: Antibody A (Capture Antibody)/Antibody B (Detection Antibody); Reagent conditions: 0.8% solids, 250 μg/mL Antibody Alkaline Phosphatase conjugate; and Sample Inlet Print: GFAP specific. The assay time was 10-15 min (with 7-12 min sample capture time).

### Example 3

Prototype i-STAT UCH-L1 assays and the i-STAT GFAP assays for Alinity were conducted using various concentrations of MgCl₂ solutions with and without heparin and assay performance on whole blood was evaluated. Solutions containing a nuclease with and without heparin were also tested to evaluate impact on assay performance using whole blood.

FIG. 1 shows screening of donor samples using solutions containing various concentrations of nuclease with or without heparin to determine which solution effectively mitigates non-specific conjugate binding (shown in FIG. 1 as GFAP_RCL-D). RCL-D as shown in FIG. 1, or reference current limit difference, is a measure of non-specific conjugate binding to the reference electrode assessed by the difference in electric current generated on the reference electrode relative to electric currents generated on reference electrodes that do not present nonspecific conjugate binding. Since RCL-D is an assay-specific number, it is designated in FIG. 1 with respect to the analyte GFAP, hence “GFAP_RCL-D”. Equivalent results were obtained by analyzing the reference currents obtained on the reference electrode for the UCH-L1 assay (data not shown for UCH-L1_RCL-D).

Staining studies confirmed that addition of nuclease prevents the formation of DNA aggregates (data not shown), and additional studies with DNase confirmed that that aggregates are composed of DNA (data not shown). The nuclease used for the results shown in FIG. 1 was Benzonase® Nuclease (Millipore Sigma). Subsequent experiments have demonstrated similar results for Deoxyribonuclease 1, from bovine pancreas (Sigma, Product Number D5025), and Turbonuclease® (Sigma, Product Number T4330). As shown in FIG. 1, concentrations of at least 1 U/mL effectively mitigate non-specific conjugate binding and the formation of DNA aggregates in the cartridge. Nuclease does not impact assay precision.

### Example 4

Three prototype cartridges were constructed to perform the i-STAT UCH-L1 assays and the i-STAT GFAP assays described in Examples 1 and 2 using an i-STAT point-of-care device. Each cartridge can perform an i-STAT UCH-L1 assay and i-STAT GFAP assay simultaneously. The cartridges were identical except for the differences described below:

Cartridge #1: Pre-treatment reagents printed in the sample chamber: salts, anti-IgG antibodies, anti-IgM antibodies and 1.5 U/mL Benzonase® nuclease.

Cartridge #2: Pre-treatment reagents printed in the sample chamber: salts, anti-IgG antibodies, anti-IgM antibodies, 1.5 U/mL Benzonase® nuclease, and 42 mM MgCl₂.

Cartridge #3: Pre-treatment reagents printed in the sample chamber: salts, anti-IgG antibodies, and anti-IgM antibodies.

Whole blood samples were collected from 48 subjects who suffered a TBI. Two cartridges were tested per patient sample. An in-house fluorescence staining methodology was used to confirm the identity of the aggregates as DNA.

The results showed that the samples tested with cartridge #3 exhibited detection errors due to the formation of nucleic acid strands on the capture and reference sensors in the cartridge. Samples tested with cartridge #1 exhibited no detection errors and the samples tested with cartridge #2 also exhibited detection errors, although at a rate lower than cartridge #3.

### Example 5

Three prototype cartridges were constructed to perform the i-STAT UCH-L1 assays and the i-STAT GFAP assays described in Examples 1 and 2 using an i-STAT point-of-care device. Each cartridge can perform an i-STAT UCH-L1 assay and i-STAT GFAP assay simultaneously. The cartridges were identical except for the differences described below:

Cartridge #1: Pre-treatment reagents printed in the sample chamber: salts, anti-IgG antibodies, anti-IgM antibodies and 1.5 U/mL Benzonase® nuclease.

Cartridge #2: Pre-treatment reagents printed in the sample chamber: salts, anti-IgG antibodies, anti-IgM antibodies, 1.5 U/mL Benzonase® nuclease, and 42 mM MgCl₂.

Cartridge #3: Pre-treatment reagents printed in the sample chamber: salts, anti-IgG antibodies, and anti-IgM antibodies.

Whole blood samples were collected from 195 subjects in various parts of The Ottawa Hospital in Canada. The samples collected included those from subjects having a variety of injuries and/or health issues, such as, head, brain or neck injuries, heart issues, various issues including pain, poisoning, COVID-19, bacterial infections, convulsions, unspecified injuries and unspecified aneurysms. Two cartridges were tested per patient sample.

The results showed that the samples tested with cartridge #3 exhibited detection errors due to the formation of nucleic acid strands on the capture and reference sensors in the cartridge. Samples tested with cartridges #1 and #2 exhibited no detection errors.

In addition, these results also show that traumatic brain injury was not required to produce the aggregates or errors in the results. Other injuries and diseases also produced the aggregates and error in the results.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A cartridge for use in a point-of-care device, the cartridge comprising a housing, wherein the housing comprises a pre-treatment reagent for use in performing at least one assay, and further wherein the pre-treatment reagent comprises at least one nuclease.

Clause 1A. The cartridge of clause 1, wherein the point-of-care device comprises a housing comprising the pre-treatment reagent.

Clause 2. The cartridge of clause 1, wherein the housing further comprises a biosensor chip, a sample chamber, and a waste container.

Clause 3. The cartridge of clause 2, wherein the pre-treatment reagent comprises a printed pre-treatment layer in the sample chamber.

Clause 4. The cartridge of clause 3, wherein the pre-treatment layer comprises about 1.5 U/mL of a nuclease.

Clause 4A. The cartridge of clause 3, wherein the pre-treatment layer further comprises one or more salts.

Clause 4B. The cartridge of clause 4A, wherein the one or more salts is magnesium chloride.

Clause 4C. The cartridge of any one of claims 1-4B, wherein the nuclease is an endonuclease.

Clause 5. The cartridge of any of clauses 1-4C, wherein the nuclease is a Serratia marcescens nuclease.

Clause 6. The cartridge of clause 4 or clause 5, wherein the pre-treatment reagent dissolves when contacted with a biological sample containing an biomarker or analyte of interest.

Clause 6A. The cartridge of clause 4 or clause 5, wherein the biological sample is a whole blood sample.

Clause 6B. The cartridge of clause 4 or clause 6, wherein the biological sample is a serum sample.

Clause 6C. The cartridge of clause 4 or clause 5, wherein the biological sample is a plasma sample.

Clause 7. The cartridge of clause 6, wherein the amount of nuclease dissolved in the biological sample is about 1.0 U/mL.

Clause 8. The cartridge of clause 6 or clause 7, wherein the biomarker or analyte of interest is GFAP, UCH-L1, or GFAP and UCH-L1.

Clause 8A. The cartridge of clause 6 or clause 7, wherein the biomarker or analyte of interest is GFAP.

Clause 8B. The cartridge of clause 6 or clause 7, wherein the biomarker or analyte of interest is UCH-L1.

Clause 8C. The cartridge of clause 6 or clause 7, wherein the biomarker or analyte of interest is GFAP and UCH-L1.

Clause 9. A method of measuring a level of a biomarker in a sample, the method comprising performing at least one assay comprising the following steps:
a) contacting a sample obtained from a subject, either simultaneously or sequentially, in any order, with: (1) one or more capture antibodies which bind to an epitope on the biomarker to form a capture antibody-biomarker-complex, (2) one or more detection antibodies which bind to an epitope on the biomarker that is not bound by the capture antibody to form a capture antibody-biomarker-detection antibody complex, wherein the detection antibody comprise a detectable label, and (3) a solution comprising at least one nuclease; and
b) measuring an amount or concentration of the biomarker in the sample based upon a signal generated by the detectable label in the capture antibody-biomarker-detection antibody complex,
wherein the biomarker is glial fibrillary acid protein (GFAP), ubiquitin carboxy-terminal hydrolase L1 (UCH-L1), or GFAP and UCH-L1, and contacting the sample with the solution comprising at least one nuclease (i) reduces non-specific conjugate binding during the assay, (ii) reduces formation of aggregates during the assay, or (iii) a combination of (i) and (ii), when compared to a corresponding method conducted without the solution comprising at least one nuclease.

Clause 9A. The method of clause 9, wherein contacting the sample with the solution comprising at least one nuclease reduces non-specific conjugate binding during the assay.

Clause 9B. The method of clause 9, wherein contacting the sample with the solution comprising at least one nuclease reduces formation of aggregates.

Clause 9C. The method of clause 9, wherein contacting the sample with the solution comprising at least one nuclease reduces non-specific conjugate binding during the assay and reduces formation of aggregates during the assay when compared to a corresponding method conducted without the solution comprising at least one nuclease.

Clause 10. The method of clause 9, comprising contacting the sample with the solution comprising at least one nuclease:
a) prior to contacting the sample with the capture antibody or the detection antibody;
b) after contacting the sample with the capture antibody; or
c) after contacting the sample with the capture antibody and the detection antibody.

Clause 10A. The method of clause 9, comprising contacting the sample with the solution comprising at least one nuclease prior to contacting the sample with the capture antibody.

Clause 10B. The method of clause 9, comprising contacting the sample with the solution comprising at least one nuclease prior to contacting the sample with the detection antibody.

Clause 10C. The method of clause 9, comprising contacting the sample with the solution comprising at least one nuclease after contacting the sample with the capture antibody.

Clause 10D. The method of clause 9, comprising contacting the sample with the solution comprising at least one nuclease after contacting the sample with the capture antibody and the detection antibody.

Clause 11. The method of clause 9 or clause 10, wherein the biomarker is a TBI biomarker.

Clause 12. The method of any one of clauses 9-11, wherein the at least one nuclease is an endonuclease.

Clause 12A. The method of any one of clauses 9-11, wherein the at least one nuclease is a Serratia marcescens nuclease.

Clause 12B. The method of any of clauses 1-12A, wherein solution containing the nuclease further comprises at least one salt.

Clause 12C. The method of clause 12B, wherein the salt is magnesium chloride.

Clause 13. The method of any one of clauses 9-12, wherein the at least one nuclease cleaves DNA, RNA, or DNA and RNA.

Clause 14. The method of any one of clauses 9-13, wherein the solution comprising the at least one nuclease comprises at least about 1 U/mL of the nuclease.

Clause 15. The method of any one of clauses 9-14, wherein the solution comprising the at least one nuclease comprises from about 1 U/mL to about 1.5 U/mL of the nuclease.

Clause 16. The method of any one of clauses 9-15, wherein the solution comprising the at least one nuclease does not contain heparin.

Clause 16A. The method of any of clauses 9-15, wherein the method does not use any heparin.

Clause 17. The method of any one of clauses 9-16, wherein the at least one assay is performed using a point-of-care device.

Clause 18. The method of any one of clauses 9-17, wherein the sample is a whole blood sample.

Clause 18A. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a whole blood sample.

Clause 18B. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a plasma sample.

Clause 18C. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a serum sample.

Clause 18D. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a whole blood sample.

Clause 18E. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a plasma sample.

Clause 18F. The method of any one of clauses 9-17, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a serum sample.

Clause 19. In an improvement of an assay for measuring an amount of a biomarker for traumatic brain injury (TBI) in a sample obtained from a subject, wherein said improvement comprises using a solution containing at least one nuclease when performing the assay and further wherein the TBI biomarker is glial fibrillary acid protein (GFAP), ubiquitin carboxy-terminal hydrolase L1 (UCH-L1), or GFAP and UCH-L1.

Clause 20. A kit comprising a capture antibody, a detection antibody, and a solution that comprises at least one nuclease, wherein use of the kit for an assay for detecting one or more biomarkers reduces (i) non-specific conjugate binding during the assay, (ii) formation of aggregates during the assay; or (iii) a combination of (i) and (ii) when compared to an assay conducted using a kit that does not comprise the at least one nuclease, and further wherein the biomarker is glial fibrillary acid protein (GFAP), ubiquitin carboxy-terminal hydrolase L1 (UCH-L1), or GFAP and UCH-L1, when compared to a corresponding assay conducted without at least one nuclease.

Clause 20A. The kit of clause 20 for use in the method of any one of clauses 1-19.

Clause 21. The kit of clause 20, wherein the solution comprising the at least one nuclease does not contain heparin.

Clause 22. A method of measuring a level of a biomarker in a sample, the method comprising performing at least one assay comprising the following steps:
a) contacting a sample obtained from a subject, either simultaneously or sequentially, in any order, with: (1) one or more capture antibodies which bind to an epitope on the biomarker to form a capture antibody-biomarker-complex, (2) one or more detection antibodies which bind to an epitope on the biomarker that is not bound by the capture antibody to form a capture antibody-biomarker-detection antibody complex, wherein the detection antibody comprise a detectable label, and (3) a solution comprising at least one nuclease; and
b) measuring an amount or concentration of the biomarker in the sample based upon a signal generated by the detectable label in the capture antibody-biomarker-detection antibody complex,
wherein contacting the sample with the solution comprising at least one nuclease (i) reduces non-specific conjugate binding during the assay, (ii) reduces formation of aggregates during the assay, or (iii) a combination of (i) and (ii), when compared to a corresponding method conducted without the solution comprising at least one nuclease.

Clause 22A. The method of clause 22, wherein contacting the sample with the solution comprising at least one nuclease reduces non-specific conjugate binding during the assay.

Clause 22B. The method of clause 22, wherein contacting the sample with the solution comprising at least one nuclease reduces formation of aggregates, when compared to a corresponding method conducted without the solution comprising at least one nuclease.

Clause 22C. The method of clause 22, wherein contacting the sample with the solution comprising at least one nuclease reduces non-specific conjugate binding during the assay and reduces formation of aggregates during the assay, when compared to a corresponding method conducted without the solution comprising at least one nuclease.

Clause 23. The method of clause 22, comprising contacting the sample with the solution comprising at least one nuclease:
a) prior to contacting the sample with the capture antibody or the detection antibody;
b) after contacting the sample with the capture antibody; or
c) after contacting the sample with the capture antibody and the detection antibody.

Clause 23A. The method of clause 22, comprising contacting the sample with the solution comprising at least one nuclease prior to contacting the sample with the capture antibody.

Clause 23B. The method of clause 22, comprising contacting the sample with the solution comprising at least one nuclease prior to contacting the sample with the detection antibody.

Clause 23C. The method of clause 22, comprising contacting the sample with the solution comprising at least one nuclease after contacting the sample with the capture antibody.

Clause 23D. The method of clause 22, comprising contacting the sample with the solution comprising at least one nuclease after contacting the sample with the capture antibody and the detection antibody.

Clause 24. The method of clause 22 or clause 23, wherein the biomarker is a biomarker for ABI or a biomarker for TBI.

Clause 25. The method of any one of clauses 22-24, wherein the at least one nuclease is an endonuclease.

Clause 25A. The method of any one of clauses 22-24, wherein the at least one nuclease is a Serratia marcescens nuclease.

Clause 25B. The method of any of clauses 22-25A, wherein solution containing the nuclease further comprises at least one salt.

Clause 25C. The method of clause 35B, wherein the salt is magnesium chloride.

Clause 26. The method of any one of clauses 22-25, wherein the at least one nuclease cleaves DNA, RNA, or DNA and RNA.

Clause 27. The method of any one of clauses 22-26, wherein the solution comprising the at least one nuclease comprises at least about 1 U/mL of the nuclease.

Clause 28. The method of any one of clauses 22-27, wherein the solution comprising the at least one nuclease comprises from about 1 U/mL to about 1.5 U/mL of the nuclease.

Clause 29. The method of any one of clauses 22-28, wherein the solution comprising the at least one nuclease does not contain heparin.

Clause 30. The method of any one of clauses 22-29, wherein the at least one assay is performed using a point-of-care device.

Clause 31. The method of any one of clauses 22-30, wherein the sample is a whole blood sample.

Clause 31A. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a whole blood sample.

Clause 31B. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a plasma sample.

Clause 31C. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; and (iii) the sample is a serum sample.

Clause 31D. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a whole blood sample.

Clause 31E. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a plasma sample.

Clause 31F. The method of any one of clauses 22-30, wherein: (i) the method comprises contacting the sample with the solution comprising at least one nuclease prior to contacting the sample with the capture antibody or the detection antibody; (ii) the at least one nuclease is an endonuclease that cleaves DNA; (iii) the solution comprising the at least one nuclease does not contain heparin; and (iv) the sample is a serum sample.

Clause 32. The method of any one of clauses 22-31, wherein the biomarker is glial fibrillary acid protein (GFAP).

Clause 33. The method of any of clauses 22-31, wherein the biomarker is ubiquitin carboxy-terminal hydrolase L1 (UCH-L1).

Clause 34. The method of any of clauses 22-31, wherein the biomarker is glial fibrillary acid protein (GFAP) and ubiquitin carboxy-terminal hydrolase L1 (UCH-L1).

Clause 35. In an improvement of an assay for measuring an amount of a biomarker in a sample obtained from a subject, said improvement comprises using a solution containing at least one nuclease when performing the assay.

Clause 36. In an improvement of an assay for measuring an amount of a biomarker for an acquired brain injury (ABI) in a sample obtained from a subject, wherein said improvement comprises using a solution containing at least one nuclease when performing the assay.

Clause 37. In the improvement of clause 36, the biomarker is a biomarker for traumatic brain injury.

Clause 37A. In the improvement of clause 36, wherein the at least one nuclease is an endonuclease.

Clause 37B. In the improvement of clause 36, wherein the at least one nuclease is a Serratia marcescens nuclease.

Clause 38. In the improvement of clause 36 or clause 37, wherein the biomarker is glial fibrillary acid protein (GFAP), ubiquitin carboxy-terminal hydrolase L1 (UCH-L1), or GFAP and UCH-L1.

Clause 39. A kit comprising a capture antibody, a detection antibody, and a solution that comprises at least one nuclease, wherein use of the kit for an assay for detecting one or more biomarkers reduces (i) non-specific conjugate binding during the assay, (ii) formation of aggregates during the assay; or (iii) a combination of (i) and (ii) when compared to an assay conducted using a kit that does not comprise the at least one nuclease, when compared to a corresponding an assay conducted without at least one nuclease.

Clause 39A. The kit of clause 39, wherein use of the kit for detecting one or more biomarkers reduces non-specific conjugate binding during the assay.

Clause 39B. The kit of clause 39, wherein use of the kit for detecting one or more biomarkers reduces formation of aggregates.

Clause 39C. The kit of clause 39, use of the kit for detecting one or more biomarkers reduces non-specific conjugate binding during the assay and reduces formation of aggregates during the assay.

Clause 39C. The kit of any of clause 39, wherein the kit is used in the method of any of clauses 22-34.

Clause 40. The kit of clause 39, wherein the solution comprising the at least one nuclease does not contain heparin.

Clause 40A. The kit of clause 39, wherein the at least one nuclease is an endonuclease.

Clause 40B. The kit of clause 39, wherein the at least one nuclease is a Serratia marcescens nuclease.

Clause 40C. The method of any of clauses 39-40B, wherein solution containing the nuclease further comprises at least one salt.

Clause 40D. The method of clause 40C, wherein the salt is magnesium chloride.

Clause 41. The kit of clause 39, wherein the one or more biomarkers is a biomarker of acquired brain injury.

Clause 42. The kit of any one of clauses 39-41, wherein the one or more biomarkers is glial fibrillary acid protein (GFAP).

Clause 43. The kit of any one of clauses 39-41, wherein the one or more biomarkers is ubiquitin carboxy-terminal hydrolase L1 (UCH-L1).

Clause 44. The kit of any one of clauses 39-41, wherein the one or more biomarkers is glial fibrillary acid protein (GFAP) and ubiquitin carboxy-terminal hydrolase L1 (UCH-L1).

Clause 45. Use of a nuclease in preparation of a kit for measuring a level of a biomarker in a biological sample.

Clause 46. The use of clause 45, wherein the biomarker is glial fibrillary acid protein (GFAP).

Clause 47. The use of clause 45, wherein the biomarker is ubiquitin carboxy-terminal hydrolase L1 (UCH-L1).

Clause 48. The use of clause 45, wherein the biomarker is glial fibrillary acid protein (GFAP) and ubiquitin carboxy-terminal hydrolase L1 (UCH-L1).

Clause 49. The use of any of clauses 45-48, wherein the nuclease used to prepare the kit is in the form of a solution.

Clause 50. The use of clause 49, wherein the solution contains at least 1 U/mL of the nuclease.

Clause 51. The use of any of clauses 49-51, wherein the solution comprises about 1 U/mL to about 1.5 U/mL of the nuclease.

Clause 51A. The method of any of clauses 49-51, wherein solution containing the nuclease further comprises at least one salt.

Clause 51B. The method of clause 51A, wherein the salt is magnesium chloride.

Clause 52. The use of any of clauses 45-51, wherein the at least one nuclease is an endonuclease.

Clause 52A. The use of any of clauses 45-51, wherein the at least one nuclease is a Serratia marcescens nuclease.

Clause 53. A computer readable storage medium having stored thereon a computer program, wherein the computer program implements steps of a method of measuring an amount or level of at least one a biomarker in a sample when executed by a processor, wherein the method comprises:
a) acquiring signal data generated by contacting a sample obtained from a subject, either simultaneously or sequentially, in any order, with: (1) one or more capture antibodies which bind to an epitope on the at least one biomarker to form a capture antibody-biomarker-complex; (2) one or more detection antibodies which bind to an epitope on the biomarker that is not bound by the capture antibody to form a capture antibody-biomarker-detection antibody complex; and (3) a solution comprising at least one nuclease; and
b) calculating an amount or level of the biomarker in the sample based upon the data from step a).

Clause 54. The computer readable storage medium of clause 53, wherein the biomarker is glial fibrillary acid protein (GFAP).

Clause 55. The computer readable storage medium of clause 53, wherein the biomarker is ubiquitin carboxy-terminal hydrolase L1 (UCH-L1).

Clause 56. The computer readable storage medium of clause 53, wherein the biomarker is and further wherein the biomarker is glial fibrillary acid protein (GFAP) and ubiquitin carboxy-terminal hydrolase L1 (UCH-L1).

Clause 57. The computer readable storage medium of any of clauses 53-56, wherein the solution contains at least 1 U/mL of the nuclease.

Clause 58. The computer readable storage medium of clause 57, wherein the solution comprises about 1 U/mL to about 1.5 U/mL of the nuclease.

Clause 59. The computer readable storage medium of any of clauses 53-58, wherein the at least one nuclease is an endonuclease.

Clause 59A. The computer readable storage medium of any of clauses 53-58, wherein the at least one nuclease a Serratia marcescens nuclease.

Clause 59B. The computer readable storage medium of any of clauses 53-59A, wherein solution containing the nuclease further comprises at least one salt.

Clause 59C. The computer readable storage medium of clause 59B, wherein the salt is magnesium chloride.

Clause 60. A computer readable storage medium having stored thereon a computer program, wherein the computer program implements steps of a method of an assay for measuring an amount or level of at least one biomarker in a sample when executed by a processor, wherein the method comprises the steps of:
a) acquiring signal data generated by a solution comprising at least one nuclease when performing the assay; and
b) calculating an amount or level of the at least one biomarker in the sample based upon the data from step a).

Clause 61. The computer readable storage medium of clause 60, wherein the biomarker is glial fibrillary acid protein (GFAP).

Clause 62. The computer readable storage medium of clause 60, wherein the biomarker is ubiquitin carboxy-terminal hydrolase L1 (UCH-L1).

Clause 63. The computer readable storage medium of clause 60, wherein the biomarker is and further wherein the biomarker is glial fibrillary acid protein (GFAP) and ubiquitin carboxy-terminal hydrolase L1 (UCH-L1).

Clause 64. The computer readable storage medium of any of clauses 60-63, wherein the solution contains at least 1 U/mL of the nuclease.

Clause 65. The computer readable storage medium of clause 64, wherein the solution comprises about 1 U/mL to about 1.5 U/mL of the nuclease.

Clause 66. The computer readable storage medium of any of clauses 60-65, wherein the at least one nuclease is an endonuclease.

Clause 67. The computer readable storage medium of any of clauses 60-65, wherein the at least one nuclease is a Serratia marcescens nuclease.

Clause 68. The computer readable storage medium of any of clauses 60-67, wherein solution containing the nuclease further comprises at least one salt.

Clause 69. The computer readable storage medium of clause 68, wherein the salt is magnesium chloride.

What is claimed is:

1. A cartridge for use in a point-of-care device, the cartridge comprising a housing, wherein the housing comprises a pre-treatment reagent for use in performing at least one assay, and further wherein the pre-treatment reagent comprises at least one nuclease.

2. The cartridge of claim 1, wherein the housing further comprises a biosensor chip, a sample chamber, and a waste container.

3. The cartridge of claim 2, wherein the pre-treatment reagent comprises a printed pre-treatment layer in the sample chamber.

4. The cartridge of claim 3, wherein the pre-treatment layer comprises about 1.5 U/mL of a nuclease.

5. The cartridge of claim 4, wherein the nuclease is a Benzonase® nuclease.

6. The cartridge of claim 4, wherein the pre-treatment reagent dissolves when contacted with a biological sample containing a biomarker or analyte of interest.

7. The cartridge of claim 6, wherein the amount of nuclease dissolved in the biological sample is about 1.0 U/mL.

8. The cartridge of claim 6, wherein the biomarker or analyte of interest is GFAP, UCH-L1, or GFAP and UCH-L1.

9. A method of measuring a level of a biomarker in a sample, the method comprising performing at least one assay comprising the following steps:
a) contacting a sample obtained from a subject, either simultaneously or sequentially, in any order, with: (1) one or more capture antibodies which bind to an epitope on the biomarker to form a capture antibody-biomarker-complex, (2) one or more detection antibodies which bind to an epitope on the biomarker that is not bound by the capture antibody to form a capture antibody-biomarker-detection antibody complex, wherein the detection antibody comprise a detectable label, and (3) a solution comprising at least one nuclease; and
b) measuring an amount or concentration of the biomarker in the sample based upon a signal generated by the detectable label in the capture antibody-biomarker-detection antibody complex,
wherein the biomarker is glial fibrillary acid protein (GFAP), ubiquitin carboxy-terminal hydrolase L1 (UCH-L1), or GFAP and UCH-L1, and contacting the sample with the solution comprising at least one nuclease (i) reduces non-specific conjugate binding during the assay, (ii) reduces formation of aggregates during the assay, or (iii) a combination of (i) and (ii).

10. The method of claim 9, comprising contacting the sample with the solution comprising at least one nuclease:
a) prior to contacting the sample with the capture antibody or the detection antibody;
b) after contacting the sample with the capture antibody; or
c) after contacting the sample with the capture antibody and the detection antibody.

11. The method of claim 9, wherein the biomarker is a TBI biomarker.

12. The method of claim 9, wherein the at least one nuclease is an endonuclease.

13. The method of claim 9, wherein the at least one nuclease cleaves DNA, RNA, or DNA and RNA.

14. The method of claim 9, wherein the solution comprising the at least one nuclease comprises at least about 1 U/mL of the nuclease.

15. The method of claim 9, wherein the solution comprising the at least one nuclease comprises from about 1 U/mL to about 1.5 U/mL of the nuclease.

16. The method of claim 9, wherein the solution comprising the at least one nuclease does not contain heparin.

17. The method of claim 9, wherein the at least one assay is performed using a point-of-care device.

18. The method of claim 9, wherein the sample is a whole blood sample.

19. In an improvement of an assay for measuring an amount of a biomarker for traumatic brain injury (TBI) in a sample obtained from a subject, wherein said improvement comprises using a solution containing at least one nuclease when performing the assay and further wherein the TBI biomarker is glial fibrillary acid protein (GFAP), ubiquitin carboxy-terminal hydrolase L1 (UCH-L1), or GFAP and UCH-L1.

20. A kit comprising a capture antibody, a detection antibody, and a solution that comprises at least one nuclease, wherein use of the kit for an assay for detecting one or more biomarkers reduces (i) non-specific conjugate binding during the assay, (ii) formation of aggregates during the assay; or (iii) a combination of (i) and (ii) when compared to an assay conducted using a kit that does not comprise the at least one nuclease, and further wherein the biomarker is glial fibrillary acid protein (GFAP), ubiquitin carboxy-terminal hydrolase L1 (UCH-L1), or GFAP and UCH-L1.

21. The kit of claim 20, wherein the solution comprising the at least one nuclease does not contain heparin.


SEQUENCE LISTING (5 sequences)

SEQ ID NO: 1 - protein, 223 aa, synthetic construct [sequence omitted]

SEQ ID NO: 2 - protein, 432 aa, synthetic construct
  REGION 1..432: note = Synthetic
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 3 - protein, 6 aa, synthetic construct
  REGION 1..6: note = Synthetic
HHHHHH

SEQ ID NO: 4 - protein, 5 aa, synthetic construct
  REGION 1..5: note = Synthetic
DDDDK

SEQ ID NO: 5 - protein, 6 aa, synthetic construct
  REGION 1..6: note = Synthetic
ADDDDK